(19) United States
(12) Patent Application Publication
(10) Pub. No.: US 2019/0147982 A1
(43) Pub. Date: May 16, 2019

(54) COMPOSITIONS AND METHODS RELATED TO OBSTRUCTIVE SLEEP APNEA

(71) Applicant: The University of Chicago, Chicago, IL (US)

(72) Inventors: David Gozal, Chicago, IL (US); Lev Becker, Chicago, IL (US)

(21) Appl. No.: 16/185,641

(22) Filed: Nov. 9, 2018

Related U.S. Application Data
(63) Continuation of application No. 14/771,875, filed on Sep. 1, 2015, now abandoned.
(60) Provisional application No. 61/773,936, filed on Mar. 7, 2013.

(51) Int. Cl. G16B 40/00; G01N 30/72; G01N 33/68

(52) U.S. Cl. CPC G16B 40/00; G01N 30/7233; G01N 2800/60; G01N 2800/2864; G01N 33/6893

(57) ABSTRACT

The technology concerns methods and compositions for diagnosing obstructive sleep apnea, a common condition observed in children. In certain embodiments, there are methods and compositions relating to the use of novel biomarkers to diagnose obstructive sleep apnea.

### CROSS-REFERENCE TO RELATED APPLICATIONS

This application is a continuation of U.S. patent application Ser. No. 14/771,875 filed Sep. 1, 2015, which is a national phase application under 35 U.S.C. § 371 of International Application No. PCT/US2014/021750 filed Mar. 7, 2014, which claims the benefit of priority of U.S. Provisional Application No. 61/773,936, filed Mar. 7, 2013. The entire contents of each of the above-referenced disclosures are specifically incorporated herein by reference without disclaimer.

### BACKGROUND OF THE INVENTION

### I. Field of the Invention

The present invention relates generally to the field of obstructive sleep apnea. More particularly, it concerns the methods and compositions for diagnosing obstructive sleep apnea.

### II. Description of the Related Art

Obstructive sleep apnea (OSA) is a prevalent disorder affecting up to 2-3% of children. It imposes substantial neurocognitive, behavioral, metabolic, and cardiovascular morbidities (Lumeng and Chervin, 2008; Capdevila et al., 2008). This condition is characterized by repeated events of partial or complete obstruction of the upper airways during sleep, leading to recurring episodes of hypercapnia, hypoxemia, and arousal throughout the night (Muzumdar and Arens, 2008). Pediatric sleep apnea is a common disorder primarily caused by enlarged tonsils and adenoids impinging upon the patency of the upper airway during sleep. Mechanisms leading to the proliferation and enlargement of the tonsils and adenoids in children who subsequently develop obstructive sleep apnea remain unknown. Adenotonsillar hypertrophy is the major pathophysiological contributor to OSA in children (Arens et al., 2003; Katz and D'Ambrosio, 2008). However, the mechanisms underlying the regulation of benign follicular lymphoid proliferation, hypertrophy, and hyperplasia are poorly understood, severely limiting the prediction of children who are at risk for developing adenotonsillar enlargement and OSA. Several epidemiological studies have demonstrated that factors such as environmental smoking, allergies, and intercurrent respiratory infections are associated with either transient or persistent hypertrophy of lymphadenoid tissue in the upper airways of snoring children (Kaditis et al., 2004; Teculescu et al., 1992; Ersu et al., 2004). Interestingly, all of these risk factors involve the generation of an inflammatory response, suggesting that the latter may promote the onset and maintenance of proliferative signals to lymphadenoid tissues.

The gold standard diagnostic approach for OSA is an overnight sleep study, or polysomnography. While this approach is reliable, it suffers from problems associated with its implementation in the clinical setting. Indeed, polysomnography is labor intensive, inconvenient, and expensive resulting in long waiting periods and unnecessary delays in diagnosis and treatment. Therefore, novel, diagnostic strategies are needed.

### SUMMARY OF THE INVENTION

Embodiments concern compositions and methods that provide diagnostic applications for addressing OSA.

In some aspects, embodiments provide a method for identifying a subject as having obstructive sleep apnea (OSA) comprising measuring from a biological sample from the subject the expression levels of one or more proteins encoded by one ore more genes listed in Table 1, and identifying the subject as having OSA based on the levels of expression of the one or more proteins. In some embodiments, the method comprises comparing the level of expression of the one or more proteins to a control or reference level. In some embodiments, an elevated level of expression of the one or more proteins as compared to a control or reference level indicates that the subject is likely to have OSA. In some embodiments, a lower level of expression of the one or more proteins as compared to a control or reference level indicates that the subject is likely to have OSA. The control may be any appropriate standard. In some embodiments, the control is the level of expression of the one or more proteins in a control sample from a subject who is known not to have OSA. In some embodiments, the level of expression of the one or more proteins is standardized against the level of expression of a corresponding standard protein in the sample. In some embodiments, the standard protein is a protein encoded by one or more genes listed in Table 1.

In some embodiments, the level of expression is measured for at least 2, 3, 4, 5, 6, 7, 8, 9, or 10 proteins. In some embodiments, the one or more proteins are encoded by a gene listed in Table 1. In some embodiments, the one or more proteins are encoded by a gene selected from the group consisting of CD14, CTSB, HPX, DPP4, TTR, DEFB1|HBD1, FABP3, CP, and AZGP1. In some embodiments, the one or more proteins are encoded by one or more genes selected from the group consisting of HPX, DPP4, CP, and AZGP1.

In some embodiments, the method further comprises obtaining the biological sample from the subject. The sample may be any appropriate sample. In some embodiments, the sample is a urine sample. In some embodiments, the corresponding standard protein is urinary creatinine. In some embodiments, the sample may be collected at a particular time of day. In some embodiments, the sample is collected in the morning, which means before 12 p.m. In certain embodiments, the sample is collected within 1 or 2 hours of waking up. In some embodiments, the sample is collected in the evening. In some embodiments, the sample is collected in the evening, which means after 4 p.m. for the subject. In other embodiments, the sample is collected after the subject has been awake for at least 8 hours or for at least 12 hours. In some embodiments, the sample is collected after the subject has been awake less than 1 hour. In some embodiments, the subject is suspected of having OSA.

In some embodiments, the subject is a male. In some embodiments, the control is the level of expression of the one or more proteins in a control male. In some embodiments, the control male is known to have OSA. In some embodiments, the control male is known to not have OSA. In some embodiments, the subject is a female. In some embodiments, the control is the level of expression of the one or more proteins in a control female. In some embodiments, the control female is known to have OSA. In some embodiments, the control female is known to not have OSA.

In some embodiments, the method further comprises using a computer algorithm to evaluate the measured levels of expression of one or more genes from Table 1. In some embodiments, the method further comprises determining a risk score for the subject for having OSA. In some embodiments, the method further comprises measuring the expression levels of RNA transcripts. In some embodiments, the expression levels of RNA transcripts are measured using DNA complementary to the RNA transcripts. In some embodiments, expression levels of RNA transcripts are measured using an amplification or hybridization assay. In some embodiments, expression levels of proteins are measured. In some embodiments, expression levels of proteins are measured using one or more binding polypeptides. In some embodiments, one or more binding polypeptides is an antibody.

In some embodiments, the method further comprises performing a sleep study on the subject. In some embodiments, the sleep study comprises one of more of the following: using a polysomnogram (PSG), performing a multiple sleep latency test (MSLT), or performing a maintenance of wakefulness test (MWT). In some embodiments, the sleep study comprises measuring one or more physiological characteristics of the subject when sleeping. In some embodiments, the physiological characteristics include one or more of the following: brain activity, heart rate, heart rhythm, blood pressure, exhaled carbon dioxide in breath, and oxygen content in blood. In some embodiments, the sleep study comprising using an actigraph. In some embodiments, the sleep study is performed after expression levels are measured in the subject.

In some aspects, embodiments provide a method for determining whether a subject has obstructive sleep apnea (OSA) comprising assaying from a biological sample from the subject the levels of expression of one or more proteins encoded by a gene listed in Table 1, and calculating a risk score for the biological sample that identifies the likelihood of the subject having OSA. In some embodiments, calculating a risk score comprises using a computer and an algorithm. In some embodiments, calculating a risk score comprises applying model coefficients to each of the levels of expression. In some embodiments, the method further comprises identifying the patient as having a risk score indicative of 50% chance or greater of having OSA. In particular embodiments, calculating a risk score involves using or running a computer algorithm or program on a computer. In further embodiments, the risk score is reported. In further embodiments, the subject is identified as having a risk score indicative of having OSA.

In some aspects, the invention provides a method for determining whether a male subject has obstructive sleep apnea (OSA) comprising measuring from a biological sample from the subject the levels of expression of one or more proteins encoded by a gene listed in Table 1, and evaluating whether the subject has OSA based on the levels of expression of the one or more proteins. In some embodiments, the one or more proteins is encoded by a gene selected from the group consisting of DDP4, HPX, and CP. In some embodiments, the method further comprises obtaining the biological sample from the subject. The sample may be any appropriate sample. In some embodiments, the sample is a urine sample. In some embodiments, the corresponding standard protein is urinary creatinine. In some embodiments, the sample may be collected at a particular time of day. In some embodiments, the sample is collected in the morning, which means before 12 p.m. In certain embodiments, the sample is collected within 1 or 2 hours of waking up. In some embodiments, the sample is collected in the evening. In some embodiments, the sample is collected in the evening, which means after 4 p.m. for the subject. In other embodiments, the sample is collected after the subject has been awake for at least 8 hours or for at least 12 hours. In some embodiments, the sample is collected after the subject has been awake less than 1 hour. In some embodiments, the subject is suspected of having OSA. In some embodiments, a lower level of expression of the one or more proteins as compared to a control indicates that the subject is likely to have OSA. In some embodiments, the control is the level of expression of the one or more proteins in a control male. In some embodiments, the control male is known to have OSA. In some embodiments, the control male is known to not have OSA. In some embodiments, the control is the level of expression of the one or more proteins in a control female.

In some aspects, embodiments provide a method for determining whether a female subject has obstructive sleep apnea (OSA) comprising determining from a biological sample from the subject the levels of expression of one or more proteins encoded by a gene listed in Table 1, and evaluating whether the subject has OSA based on the levels of expression of the one or more proteins. In some embodiments, the one or more proteins is encoded by AZGP1. In some embodiments, the method further comprises obtaining the biological sample from the subject. The sample may be any appropriate sample. In some embodiments, the sample is a urine sample. In some embodiments, the corresponding standard protein is urinary creatinine. In some embodiments, the sample may be collected at a particular time of day. In some embodiments, the sample is collected in the morning, which means before 12 p.m. In certain embodiments, the sample is collected within 1 or 2 hours of waking up. In some embodiments, the sample is collected in the evening. In some embodiments, the sample is collected in the evening, which means after 4 p.m. for the subject. In other embodiments, the sample is collected after the subject has been awake for at least 8 hours or for at least 12 hours. In some embodiments, the sample is collected after the subject has been awake less than 1 hour. In some embodiments, the subject is suspected of having OSA. In some embodiments, an elevated level of expression of the one or more proteins as compared to a control indicates that the subject is likely to have OSA. In some embodiments, the control is the level of expression of the one or more proteins in a control female. In some embodiments, the control female is known to have OSA. In some embodiments, the control female is known to not have OSA. In some embodiments, the control is the level of expression of the one or more proteins in a control male.

In some aspects, embodiments provide a method for evaluating obstructive sleep apnea in a subject comprising subjecting the subject to a sleep study after the subject is determined to have sleep apnea based on measuring expression levels of one or more genes listed in Table 1 in a urine sample obtained from the subject. In some embodiments, the sleep study comprises one of more of the following: using a polysomnogram (PSG), performing a multiple sleep latency test (MSLT), or performing a maintenance of wakefulness test (MWT). In some embodiments, the sleep study comprises measuring one or more physiological characteristics of the subject when sleeping. In some embodiments, the physiological characteristics include one or more of the following: brain activity, heart rate, heart rhythm, blood pressure, exhaled carbon dioxide in breath, and oxygen content in blood. In some embodiments, the sleep study comprises using an actigraph.

In some aspects, provided is a method for identifying a subject as having high-risk obstructive sleep apnea (OSA) comprising a) measuring from a biological sample from the subject the expression levels of one or more products of one or more genes listed in either Table 1 or Table 2, and b) identifying the subject as having high-risk OSA based on the levels of expression of the one or more products. In some aspects, provided is a method for identifying a subject as at risk for having high-risk obstructive sleep apnea (OSA) comprising a) measuring from a biological sample from the subject the expression levels of one or more products of one or more genes listed in either Table 1 or Table 2, and b) identifying the subject as at risk for having high-risk OSA based on the levels of expression of the one or more products. High-risk OSA is understood to be OSA which is associated with neurocognitive impairment such as memory impairment, declarative memory defects, learning delays, and issues with academic performance, mood-related disorders such as depression, behavioral issues such as ADHD, aggression, inattentiveness, impulsivity, and excessive sleepiness, cardiovascular risks including hypertension, altherosclerosis, pulmonary hypertension, and left ventricular dysfunction, a metabolic disorders such as dyslipidemia and insulin resistance. In some aspects, provided is a method for identifying a subject as having an increased risk of neurocognitive impairment such as memory impairment, declarative memory defects, learning delays, and issues with academic performance, mood-related disorders such as depression, behavioral issues such as ADHD, aggression, inattentiveness, impulsivity, and excessive sleepiness, cardiovascular risks including hypertension, altherosclerosis, pulmonary hypertension, and left ventricular dysfunction, a metabolic disorders such as dyslipidemia and insulin resistance comprising a) measuring from a biological sample from the subject the expression levels of one or more products of one or more genes listed in either Table 1 or Table 2, and b) identifying the subject as having an increased risk of neurocognitive impairment such as memory impairment, declarative memory defects, learning delays, and issues with academic performance, mood-related disorders such as depression, behavioral issues such as ADHD, aggression, inattentiveness, impulsivity, and excessive sleepiness, cardiovascular risks including hypertension, altherosclerosis, pulmonary hypertension, and left ventricular dysfunction, a metabolic disorders such as dyslipidemia and insulin resistance based on the levels of expression of the one or more products.

In some embodiments, the level of expression of the one or more products is compared to a control or reference level. The control or reference level may be any appropriate level. In some embodiments, an elevated level of expression of the one or more products as compared to a control or reference level indicates that the subject is likely to have OSA with declarative memory defects. In some embodiments, a lower level of expression of the one or more products as compared to a control or reference level indicates that the subject is likely to have OSA with declarative memory defects. In some embodiments, the control is the level of expression of the one or more products in a control sample from a subject who is known not to have OSA. In some embodiments, the control is the level of expression of the one or more products in a control sample from a subject who is known to have OSA. In some embodiments, the level of expression of the one or more products is standardized against the level of expression of a corresponding standard product in the sample.

In some embodiments, the level of expression is measured for at least 2, 3, 4, 5, 6, 7, 8, 9, or 10 proteins. In some embodiments, the one or more proteins are encoded by a gene listed in either Table 1 or Table 2. In some embodiments, the one or more products are one or more proteins encoded by a gene selected from the group consisting of RNASE1, COL12A1, RNASE2, CD59, FN1, AMBP, FBN1, PIK3IP1, CDH1, CDH2, PLG, SLURP1, FN1 cDNA FLJ53292, TNC, C1RL, A1BG, PGLYRP2, OSCAR, AZGP1, CEL, CFI, CILP2, VASN, PLAU, SERPINA1, CD14, LRP2, CLU, FGA, NID1, APOD, SERPING1, CADM4, CP, IGHA1, PGLYRP1, ROBO4, SERPINA5, MASP2, HPX, IGHV4-31, IGHG1, MXRA8, AMY1C, AMY1A, AMY1B, AMY2A, COL6A1, EGF, PROCR, PIGR, ITIH4, CUBN, LMAN2, TF, and KNG1. In some embodiments, the one or more products are one or more proteins encoded by one or more genes selected from the group consisting of KNG1, PIGR, PROCR, HPX, CP, RNASE1, COL12A1, CD59, APOH, and CTBS. In some embodiments, the one or more products are one or more proteins encoded by one or more genes selected from the group consisting of HPX and CP.

In some embodiments, the method further comprises obtaining the biological sample from the subject. The sample may be any appropriate sample. In some embodiments, the sample is a urine sample. In some embodiments, the corresponding standard protein is urinary creatinine. In some embodiments, the sample may be collected at a particular time of day. In some embodiments, the sample is collected in the morning, which means before 12 p.m. In certain embodiments, the sample is collected within 1 or 2 hours of waking up. In some embodiments, the sample is collected in the evening. In some embodiments, the sample is collected in the evening, which means after 4 p.m. for the subject. In other embodiments, the sample is collected after the subject has been awake for at least 8 hours or for at least 12 hours. In some embodiments, the sample is collected after the subject has been awake less than 1 hour. In some embodiments, the subject is suspected of having OSA.

In some embodiments, the subject is known to have OSA. In some embodiments, the method further comprises identifying the subject as a candidate for evaluation by the methods disclosed herein by administration of a questionnaire. In some embodiments, the method further comprises using a computer algorithm to evaluate the measured levels of expression of one or more genes from Table 1 or Table 2. In some embodiments, the method further comprises determining a risk score for the subject for having OSA with declarative memory defects. In some embodiments, the expression levels of RNA transcripts are measured. In some embodiments, the expression levels of RNA transcripts are measured using DNA complementary to the RNA transcripts. In some embodiments, expression levels of RNA transcripts are measured using an amplification or hybridization assay. In some embodiments, expression levels of proteins are measured. In some embodiments, expression levels of proteins are measured using one of more binding polypeptides. In some embodiments, one or more binding polypeptides is an antibody. In some embodiments, the method further comprises treating the subject identified as having high-risk OSA. In some embodiments, treating the subject includes pharmacological treatment with corticosteroids, leukotriene antagonists, topical nasal steroids, intranasal steroids, and/or montelukast, surgical removal of the adenoids and tonsils, applying positive airway pressure therapy (PAP), or the application of oral applicances.

In some aspects, provided is a method for determining whether a subject has obstructive sleep apnea (OSA) with declarative memory defects comprising a) assaying from a biological sample from the subject the levels of expression of one or more proteins encoded by a gene listed in Table 1 or Table 2; and b) calculating a risk score for the biological sample that identifies the likelihood of the subject having OSA with declarative memory defects. In some embodiments, calculating a risk score comprises using a computer and an algorithm. In some embodiments, calculating a risk score comprises applying model coefficients to each of the levels of expression. In some embodiments, the method further comprises identifying the patient as having a risk score indicative of 50% chance or greater of having OSA with declarative memory defects. In some aspects, provided is a method for treating high-risk obstructive sleep apnea (OSA) in a subject comprising pharmacological treatment with corticosteroids, leukotriene antagonists, topical nasal steroids, intranasal steroids, and/or montelukast, surgical removal of the adenoids and tonsils, applying positive airway pressure therapy (PAP), or the application of oral applicances after the subject is determined to have sleep apnea based on measuring expression levels of one or more genes listed in Table 1 or Table 2 in a urine sample obtained from the subject.

In some embodiments, the subject is a child or minor. In some embodiments, the child or minor is, is at least, or is at most 1, 2, 3, 4, 5, 6, 7, 8, 9, 10, 11, 12, 13, 14, 15, 16, 17, or 18 years old.

Some methods also involve comparing the expression level of the at least one protein to the expression level of a control from the sample. In other embodiments, methods involve comparing the expression level of at least one protein to the expression level of that protein in a standardized sample. An increase or decrease in the level of expression will be evaluated. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 comparative protein (or any range derivable therein) may be used in comparisons or compared to the expression level of a protein. In other embodiments at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 comparative protein are measured. In particular embodiments, at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 comparative protein are compared to one or more proteins.

In other embodiments, a coefficient value is applied to each protein expression level. The coefficient value reflects the weight that the expression level of that particular protein has in assessing the whether or not the subject has OSA. In certain embodiments, the coefficient values for a plurality of proteins whose expression levels are measured. The plurality may be, be at least, or be at most 1, 2, 3, 4, 5, 6, 7, 8, 9, 10, 11, 12, 13, 14, 15, 16, 17, 18, 19, 20, 21, 22, 23, 24, 25, 26 of these proteins, as well as any proteins discussed herein. Methods and computer readable medium can be implemented with coefficient values.

In some embodiments, methods will involve determining or calculating a diagnostic score based on data concerning the expression level of one or more proteins, meaning that the expression level of the one or more proteins is at least one of the factors on which the score is based. A diagnostic score will provide information about the biological sample, such as the general probability that the subject has OSA. In some embodiments, the diagnostic score represents the probability that the subject has OSA or does not have OSA. In certain embodiments, a probability value is expressed as a numerical integer or number that represents a probability of 0% likelihood to 100% likelihood that OSA. In some embodiments, the probability value is expressed as a numerical integer or number that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has OSA. Alternatively, the probability may be expressed generally in percentiles, quartiles, or deciles.

In some embodiments, methods include evaluating one or more proteins using a scoring algorithm to generate a diagnostic score for OSA, wherein the patient is identified as having or as not having OSA based on the score. It is understood by those of skill in the art that the score is a predictive value about the classification of OSA. In some embodiments, a report is generated and/or provided that identifies the diagnostic score or the values that factor into such a score. In some embodiments, a cut-off score is employed to characterize a sample as likely having OSA. In some embodiments, the risk score for the patient is compared to a cut-off score to characterize the biological sample from the patient with respect to OSA. In certain embodiments, the diagnostic score is calculated using a weighted coefficient for each of the measured protein levels of expression. The weighted coefficients will typically reflect the significance of the expression level of a particular protein for determining risk of OSA.

Any of the methods described herein may be implemented on tangible computer-readable medium comprising computer-readable code that, when executed by a computer, causes the computer to perform one or more operations. In some embodiments, there is a tangible computer-readable medium comprising computer-readable code that, when executed by a computer, causes the computer to perform operations comprising: a) receiving information corresponding to a level of expression of at least one protein in a sample from a patient; and b) determining a protein expression level value using information corresponding to the at least one protein and information corresponding to the level of expression of a control. In some embodiments, receiving information comprises receiving from a tangible data storage device information corresponding to a level of expression of at least one protein in a sample from a patient. In additional embodiments, information is used that corresponds to the level of expression of a control. In additional embodiments the medium further comprises computer-readable code that, when executed by a computer, causes the computer to perform one or more additional operations comprising: sending information corresponding to the expression level of at least one protein to a tangible data storage device. In specific embodiments, it further comprises computer-readable code that, when executed by a computer, causes the computer to perform one or more additional operations comprising: sending information corresponding to the expression level of at least one protein to a tangible data storage device. In certain embodiments, receiving information comprises receiving from a tangible data storage device information corresponding to a level of expression of at least one protein in a sample from a patient. In even further embodiments, the tangible computer-readable medium has computer-readable code that, when executed by a computer, causes the computer to perform operations further comprising: c) calculating a diagnostic score for the sample, wherein the diagnostic score is indicative of the probability that the subject has OSA. It is contemplated that any of the methods described above may be implemented with tangible computer readable medium that has computer readable code, that when executed by a computer, causes the computer to perform operations related to the measuring, comparing, and/or calculating a diagnostic score related to the probability of a subject having OSA.

A processor or processors can be used in performance of the operations driven by the example tangible computer-readable media disclosed herein. Alternatively, the processor or processors can perform those operations under hardware control, or under a combination of hardware and software control. For example, the processor may be a processor specifically configured to carry out one or more those operations, such as an application specific integrated circuit (ASIC) or a field programmable gate array (FPGA). The use of a processor or processors allows for the processing of information (e.g., data) that is not possible without the aid of a processor or processors, or at least not at the speed achievable with a processor or processors. Some embodiments of the performance of such operations may be achieved within a certain amount of time, such as an amount of time less than what it would take to perform the operations without the use of a computer system, processor, or processors, including no more than one hour, no more than 30 minutes, no more than 15 minutes, no more than 10 minutes, no more than one minute, no more than one second, and no more than every time interval in seconds between one second and one hour.

Some embodiments of the present tangible computer-readable media may be, for example, a CD-ROM, a DVD-ROM, a flash drive, a hard drive, or any other physical storage device. Some embodiments of the present methods may include recording a tangible computer-readable medium with computer-readable code that, when executed by a computer, causes the computer to perform any of the operations discussed herein, including those associated with the present tangible computer-readable media. Recording the tangible computer-readable medium may include, for example, burning data onto a CD-ROM or a DVD-ROM, or otherwise populating a physical storage device with the data.

The embodiments in the Example section are understood to be embodiments of the invention that are applicable to all aspects of the invention, including compositions and methods.

The use of the word “a” or “an,” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E. Pipeline for urine biomarker discovery by LC-MS/NIS. Panel a: An optimized workflow for proteomic analysis of urine. Panels b-c: Immunoglobulin (IgG) and albumin (ALB) depletion. The extent of depletion was quantified by Bradford (Panel b) and visualized by SDS-PAGE (Panel c). Specificity of IgG and ALB removal was assessed by comparing serotransferrin (TRF) levels in depleted (+) and non-depleted (−) samples (Panel c). IgG, whole antibody; HC, heavy chain; LC, light chain; **, non-specific detection of ALB. Panel d: Urine samples were precipitated with TCA/DOC and protein levels were determined for 10 subjects. Results (N=6/subject) are displayed as box-and-Whisker plots (5-95% confidence intervals). Panel e: Gene ontology analysis of all urine proteins detected by mass spectrometry. All functional annotations presented are statistically significant (p<0.05) based on the hypergeometric test with Benjamini-Hochberg correction.

FIGS. 2A-2D. Gender and diurnal effects on the urinary proteome of healthy children. Morning (am) and bedtime (pm) urine samples were collected from healthy boys (N=7) and girls (N=6) and subjected to LC-ESI-MS/MS analysis. Proteins were quantified by spectral counting and differentially expressed proteins were detected using the t-test and G-test. Panel a: A representative statistical analysis demonstrating proteomic differences in morning samples between boys and girls. Red, up-regulated in boys; Green, down-regulated in boys. Confidence intervals (dashed lines; G>1.5 or G<−1.5 and α=0.05) and the FDR (<5%) were established by permutation analysis. Proteins that were down-regulated in boys were assigned negative values in the G-test. Panel b: A comparison of differentially expressed proteins in boys (relative to girls) in morning and bedtime samples. Panels c-d: Examples of proteins (TRF and REG1A) that are subjected to both gender and diurnal regulation. Results are means±SEMs, statistical significance (**) was assessed by a combination of the t-test and G-test.

FIGS. 3A-3E. Identification of candidate biomarkers of pediatric OSA. Morning (am) and bedtime (pm) samples were collected from children with and without OSA and subjected to LC-MS/MS. Panel a: Analysis of proteomic data was performed as follows: Level 1 (L1), morning and bedtime measurements were averaged and boys and girls were pooled; Level 2 (L2), analyses for morning and bedtime samples were conducted independently; Level 3 (L3) analyses for morning and bedtime samples were conducted independently in both boys and girls. The number of candidate biomarkers identified at each level is shown in parentheses. Panel b: Biomarkers detected in level 3 were split according to collection time and gender. Panel c: A demonstration of the “gender effect” on global proteomic analysis (based on the t-test and G-test) of morning urine samples. Red, up-regulated in OSA; green, down-regulated in OSA; dashed lines confidence intervals (FDR<5%). Panel d: Dipeptidyl peptidase 4 (DPP4) as an example of a specific biomarker for OSA in the morning samples of boys. Protein levels (mean±SEMs) were determined by spectral counting. **, statistically significant based on the t-test and G-test.

FIGS. 4A-4C. Validation of mass spectrometry data by ELISA. Differentially expressed proteins identified by proteomic analysis were validated in morning (am) and bedtime (pm) samples usin6g commercially available ELISA assays. Panel a: Comparison of hemopexin (HPX) level quantified by mass spectrometry (MS/MS) and ELISA (ng/mg creatinine). Linear regression analysis (line) detected a strong positive correlation (R²=0.52, p<0.0001) between both techniques. Panel b: Measurement of DPP4 levels by ELISA demonstrating specific down-regulation of dipeptidyl peptidase 4 (DPP4) in morning urine samples (compare to FIG. 3d). Panel c: Comparison of HPX (ng/mg creatinine), ceruloplasmin (CP; ng/mg creatinine), and zinc-α-2-glycoprotein (AZGP1; ng/mg creatinine) levels quantified by MS/MS and ELISA. Measurements were normalized relative to control samples. Where applicable results are means±SEMs. #, statistically significant based on the t-test (p<0.05) and G-test (G>1.5). **, statistically significant based on the t-test (p<0.05).

FIG. 5. Biomarkers of pediatric OSA map to pathophysiological modules. Gene ontology analysis of the 192 candidate biomarkers identified numerous functional modules enriched in children with OSA (p<0.05, hypergeometric test with Benjamini-Hochberg correction). Six representative proteins in each functional module are presented as examples.

FIGS. 6A-6D. Children with OSA exhibit heterogeneity in memory recall impairment. Healthy subjects (N=13) and children with OSA (N=20) were recruited at the University of Chicago. A: Performance on a pictoral memory recall test identified two populations of children with OSA: those with normal (OSA-N) and impaired (OSA-I) memory recall. B-D: Differences between OSA-N and OSA-I patients could not be attributed to variability in OSA severity (B), obesity (C), or age (D).

FIGS. 7A-7B. Identification of candidate urine biomarkers of memory impairment in children with OSA. Proteomics analysis of morning urine samples collected from healthy subjects (CTRL) and children with OSA that had normal (OSA-N) or impaired memory (OSA-I). A: Candidate biomarkers were identified using the t-test and G-test (red lines, confidence intervals FDR=0.1%). Yellow=up, blue=down in OSA-I versus OSA-N. B: protein abundance levels (spectral count) for two candidate biomarkers.

FIGS. 8A-8C. ELISA assays enable high throughput measurement of HPC and CP. Urinary levels of hemopexin (HPX; A), ceruloplasmin (CP; B), and uromodulin (UMOD; C) were quantified by mass spectrometry (MS/MS) and ELISA. For ELISA, values were standardized to urinary creatinine (CR) levels. Note the strong concordance between the two measures.

FIG. 9. Memory recall test: Schematic of the declarative memory test for the study. NSPG: overnight polysomnography.

### DETAILED DESCRIPTION

Obstructive sleep apnea (OSA) is a highly prevalent disorder in children (2-3%) characterized by repeated events of partial or complete upper airway obstruction during sleep. This frequent condition, which results in recurring episodes of hypercapnia, hypoxemia, and arousal throughout the night, and accrues substantially to the risk for the development of cardiovascular, metabolic, neurobehavioral, and cognitive problems.

Substantial evidence suggests that intermittent hypoxia and sleep fragmentation negatively influence academic achievement in children with OSA. Indeed, the inventors have previously demonstrated that children with OSA were more likely to display impairments in the acquisition, consolidation, or retrieval of declarative memories. Furthermore, work has identified declarative memory as a robust reporter on the presence or absence of global cognitive deficits in the context of OSA. Moreover, significant improvements in academic performance and cognitive deficits have been reported following treatment of OSA. Thus, the (early) detection of pediatric OSA patients who are predisposed to more severe memory impairment is of particular clinical significance. However, identifying children who have developed OSA-associated cognitive problems is complicated by the need for laborious neurocognitive tests that are unavailable in most clinical settings and therefore such assessments are not routinely pursued.

Intrinsic variance of the urine proteome limits the discriminative power of proteomic analysis and complicates biomarker detection. Using an optimized workflow for proteomic analysis of urine, the inventors demonstrate that gender and diurnal effects constitute two important sources of variability in healthy children. Indeed, by performing biomarker discovery in a gender and diurnal-dependent manner, the inventors identified ˜30-fold more candidate biomarkers of pediatric obstructive sleep apnea (OSA), a highly prevalent (2-3%) condition in children characterized by repetitive episodes of intermittent hypoxia and hypercapnia, and sleep fragmentation in the context of recurrent upper airway obstructive events during sleep. Remarkably, biomarkers were highly specific for gender and sampling time since poor overlap (˜3%) was observed in the proteins identified in boys and girls across morning and bedtime samples.

Since no clinical basis to explain gender-specific effects in OSA or healthy children is apparent, the data supports the implementation of contextualized biomarker strategies to a broad range of human diseases. For example, these findings indicate that aside from providing an abundant repository of disease biomarkers, the urinary proteome also comprises a wealth of information concerning disease-related pathological processes.

### A. OBSTRUCTIVE SLEEP APNEA

A person with obstructive sleep apnea (OSA) will stop breathing periodically for a short time (typically less than 60 seconds) while sleeping; it is associated with an airway that may be blocked, which prevents air from reaching the lungs. The diagnosis of this condition currently involves a physical exam and a survey about the patient's sleepiness, quality of sleep and bedtime habits. If a child is involved, questions will be posed to a parent or caregiver. A sleep study may be requested and performed to further evaluate for the presence of the condition. Other tests that may be performed include evaluation of arterial blood gases, electrocardiogram (ECG), echocardiogram, and/or thyroid function studies.

Disruption in inflammatory/immune, lipid, angiogenic, and hemostatic pathways have all been reported in patients with OSA (Adedayo, 2012; Chorostowska-Wynimko, 2005; Slupsky, 2007; von Kanel, 2007), and are proposed as the mechanistic basis for the heightened prevalence of associated co-morbidities in OSA, such as obesity, diabetes, and atherosclerosis.

OSA is a highly prevalent disease in children associated with a wide range of comorbidities. Obstructive sleep apnea (OSA) is a common disorder in children (2-3%) characterized by repeated events of partial or complete obstruction of the upper airway during sleep, resulting in recurring episodes of hypercapnia, hypoxemia, and arousal (Lumeng & Chervin, 2008). Current evidence suggests that both the sleep fragmentation, which develops as a consequence of repeated arousals, and the intermittent blood gas abnormalities (hypoxia and hypercarbia) that characterize OSA (Gozal & Kheirandish-Gozal, 2008; Kaemingk, et al., 2003; Kheirandish, et al., 2005) jointly predispose patients to a wide array of morbid consequences. The latter include reduced cognitive and academic performance and memory, behavioral deficits including attention deficit hyperactivity-like disease, aggressiveness and poor impulse control, as well as failure to thrive, enuresis and cardiovascular and metabolic dysfunction (Gozal & Kheirandish-Gozal, 2008; Gozal & Kheirandish-Gozal, 2008; Gozal, et al., 2010; Kim, et al., 2011; Spruyt, et al., 2011; Blunden, et al., 2000; Ellenbogen, et al., 2005; Gottlieb, et al., 2004; Kheirandish & Gozal, 2006; O'Brien, et al., 2003; O'Brien, et al., 2004; Rhodes, et al., 1995; Gozal, et al., 2007; Sans Capdevila, et al., 2008). Adequate treatment of OSA improves or reverses these morbidities, and is further associated with improved overall quality of life (Baldassari, et al., 2008) and reduced healthcare costs (Tarasiuk, et al., 2004).

Children with OSA exhibit reduced memory and academic performance. Preservation of both rapid eye movement (REM) sleep and non-REM sleep integrity is of great importance to the consolidation of both declarative (factual recall) and non-declarative memory (procedural skills) (Stickgold, et al., 2005). Therefore, disruption of these sleep stages may interrupt or reduce the efficacy of the processes underlying memory consolidation. In addition, sleep has been shown to strengthen memories and make them more resistant to interference in both adults (Ellenbogen, et al., 2006) and children (Hill, et al., 2007). Several studies have now shown that retention of word pairs was significantly increased after sleep, and that sleep enhanced memory performance for faces in both adults and children (Stickgold & Walker, et al., 2007; Walker & Stickgold, 2006; Backhaus, et al., 2008; Wagner, et al., 2007). Similarly, non-disrupted sleep leads to improved performance in memory recall, and enhancement of memory performance is only seen after a good night of sleep (Ellenbogen, et al., 2006; Hill, et al., 2007; Gais & Born, 2004; Ellenbogen, et al., 2006). Studies showed that children with OSA were more likely to display impairments in the acquisition, consolidation, or retrieval of memories (Kheirandish-Gozal, et al., 2010).

In addition to the diagonistic markers disclosed herein, a questionnaire may help to identify those subjects who are candidates for the methods disclosed herein. This questionnaire can request information such as the age, sex, weight, height, and race and ethnicity of the subject, in addition to more specific questions regarding the subject's sleep. Questions may include whether or not the subject stops breathing during sleep, struggles to breathe while asleep, if physical actions are ever needed to make the subject breathe again during sleep, frequency and loudness of snoring, and concerns regarding the subject's breathing while asleep. In some instances, a subject or the parent of a subject may complete such a questionnaire and, on the basis of those answers, it may be recommended that the subject be evaluated by the methods disclosed herein.

### B. BIOMARKERS AND DIAGNOSTIC METHODS

In some embodiments, there are diagnostic methods related to OSA or OSA with declarative memory defects. Diagnostic methods are based on the identification of biomarkers in a sample from a subject. A “biomarker” is a molecule useful as an indicator of a biologic state in a subject.

Genetic and environmental perturbations impose dramatic variability on protein expression patterns in individuals. Epigenetic, transcriptomic, metabolomic, and proteomic studies have highlighted the dynamics of regulation of gene expression within healthy populations (Slupsky, 2007; Christensen, 2009). For example, DNA methylation patterns in healthy human tissues were highly sensitive to age and environmental factors (Christensen, 2009). Similarly, metabolites relating to mitochondrial energy metabolism were found to differentiate gender and age in healthy adults (Slupsky, 2007). Furthermore, biomarker discovery strategies based on proteomics are complicated by low protein concentrations and high levels of interfering substances (e.g., salts and nitrogenous bases) in urine. In the context of disease, complex pathophysiological perturbations magnify these proteomic differences and therefore require contextualized biomarker analysis.

In an attempt to circumvent these problems, the inventors interrogated two important likely sources of variability (gender and diurnal effects) on both the urine proteome and biomarker discovery process of pediatric OSA. To facilitate this process, the inventors optimized a proteomics workflow for biomarker discovery based on liquid chromatography tandem mass spectrometry (LC-MS/MS), an approach that allows for deeper proteome coverage and interrogation of lower abundance proteins. Current findings demonstrate that diurnal and gender-related effects operate as powerful modulators of the urinary proteome in healthy children.

The findings demonstrate the presence of dramatic gender and diurnal effects on biomarkers of OSA, suggesting that discovery-based proteomics approaches aimed at identifying biomarkers in a contextualized manner may greatly facilitate the ability to reliably detect human disease. By incorporating these constitutive determinants of variance into the analyses, 192 putative candidate biomarkers were a priori identified in urine collected from children with OSA. Moreover, the inventors show that most if not all (˜97%) of these biomarkers retained their predictive ability only if their use was implemented in the contextual setting of their collection (i.e., morning in boys, or bedtime in girls), a result that was validated by ELISA measurements. However, some biomarkers may show their predictive ability regardless of their contextualized setting or may exhibit a different contextualized setting effect as those seen for these 97%. These results highlight the complexity of the biomarker discovery process, and suggest that carefully contextualized biomarker discovery strategies will be obligatorily needed to effectively detect human disease across broad populations.

The OSA biomarkers disclosed herein can be polypeptides that exhibit a change in expression or state, which can be correlated with the presence of OSA in a subject. The OSA biomarkers are contemplated to constitute the markers identified in Table 1. In certain embodiments, specific biomarkers in Table 1 are contemplated. In certain embodiments, 1, 2, 3, 4, 5, 6, 7, 8, 9, 10 of the biomarkers in Table 1, or a range derivable therein, may be employed in embodiments described herein. In addition, the biomarkers disclosed herein can include messenger RNAs (mRNAs) encoding the biomarker polypeptides, as measurement of a change in expression of an mRNA can be correlated with changes in expression of the polypeptide encoded by the mRNA. Changes in expression may be an increase (up-regulation) in expression in OSA cells or a decrease (down-regulation) in expression in OSA cells compared to the control cells. Whether a particular biomarker is increased or decreased is shown in Table 1. As such, determining an expression level of a gene of interest in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

TABLE 1

| IPI | UniProt | Entrez | Gene name | Description | G-test | T-test |
|---|---|---|---|---|---|---|
| IPI00032328 | P01043\|P01042\|B4E1C2\|Q7M | 3827 | KNG1 | Kininogen-1\|Kininogen 1, isoform CRA_b | 72.6 | 0.0187 |
| | 4P1\|B2RCR2\|A8K474\|Q6PA | | | | | |
| | U9\|Q53EQ0 | | | | | |
| IPI00004573 | P01833\|Q8IZY7\|Q68D81 | 5284 | PIGR | Polymeric immunoglobulin receptor | 67.3 | 0.0028 |
| IPI00220143 | Q75ME7\|Q0VAX6\|O4345\|Q | 8972 | MGAM | Maltase-glucoamylase\|Maltase- | 65.8 | 0.0279 |
| | 8TE24\|Q86UM5 | | | glucoamylase, intestinal | | |
| IPI00029260 | Q96FR6\|F1C4A7\|Q9UNS3\|Q9 | 929 | CD14 | Monocyte differentiation antigen CD14 | 57.4 | 0.0363 |
| | 6L99\|B2R888\|P08571\|Q53XT | | | | | |
| | 5 | | | | | |
| IPI00293088 | Q16302\|P10253\|Q09GN4\|Q8I | 2548 | GAA | Lysosomal alpha-glucosidase | 54.4 | 0.0356 |
| | WE7\|Q14351 | | | | | |
| IPI00014048 | Q1KHR2\|B2R589\|Q6ICS5\|Q1 | 6035 | RNASE1 | Ribonuclease pancreatic | 53.8 | 0.0034 |
| | 6869\|Q16830\|D3DS06\|P07998 | | | | | |
| | \|Q9UCB4\|Q9UCB5 | | | | | |
| IPI00291136 | Q9BSA89\|Q14040\|Q14041\|O00 | 1291 | COL6A1 | Collagen alpha-1(VI) chain\|Putative | 50.8 | 0.0024 |
| | 117\|Q16258\|O00118\|Q7Z645\| | | | uncharacterized protein | | |
| | P12109\|Q8TBN2 | | | | | |
| IPI00218192 | Q15135\|Q14624\|Q9UQ54\|Q9 | 3700 | ITIH4 | Inter-alpha-trypsin inhibitor heavy chain H4 | 48.7 | 0.0136 |
| | P190 | | | | | |
| IPI00022620 | P55000\|Q6PUA6\|Q53YJ6\|Q92 | 57152 | SLURP1 | Secreted Ly-6/uPAR-related protein 1 | 43.9 | 0.0012 |
| | 483 | | | | | |
| IPI00009950 | Q53HH1\|Q12907\|A8K7T4 | 10960 | LMAN2 | cDNA FLJ75774, highly | 41.9 | 0.0351 |
| | | | | similar to Homo sapiens | | |
| | | | | lectin, mannose-binding 2 (LMAN2), | | |
| | | | | mRNA\|Vesicular integral- | | |
| | | | | membrane protein VIP36 | | |
| IPI00294713 | Q9H498\|Q9UMV3\|Q9ULC7\| | 10747 | MASP2 | Mannan-binding lectin serine protease 2 | 34.8 | 0.0042 |
| | Q96QG4\|O75754\|Q9UC48\|O0 | | | | | |
| | 0187\|Q9H4999\|Q5TEQ5\|Q9BZ | | | | | |
| | H0\|Q5TER0\|A8K458\|A8MWJ | | | | | |
| | 2\|Q9UBP3\|Q9Y270 | | | | | |
| IPI00000073 | E9PBF0\|P01133\|B4DRK7\|Q5 | 1950 | EGF | Pro-epidermal growth factor | 30.3 | 0.0017 |
| | 2LZ6 | | | | | |
| IPI000295741 | Q6LAF9\|A8K2H4\|Q503A6\|B | 1508 | CTSB | Cathepsin B\|cDNA FLJ78235 | 30.3 | 0.0454 |
| | 3KQR5\|Q96D87\|P07858\|B3K | | | | | |
| | RR5 | | | | | |
| IPI00022488 | P02790\|B2R957 | 3263 | HPX | Hemopexin | 27.4 | 0.0086 |
| IPI00291866 | A6NMU0\|Q9UC49\|Q96FE0\|P | 710 | SERPING1 | Plasma protease C1 inhibitor\|Epididymis | 26.1 | 0.0036 |
| | 05155\|A8KAI9\|E9KL26\|Q7Z4 | | | tissue protein Li 173 | | |
| | 55\|Q16304\|B2R6L5\|Q59EI5\|Q | | | | | |
| | 547W3\|Q9UCF9 | | | | | |
| IPI00009028 | P05452\|B2R582\|Q6FGX6 | 7123 | CLEC3B | Tetranectin\|cDNA, FLJ92374, highly similar | 26.0 | 0.0014 |
| | | | | to Homo sapiens C-type lectin domain | | |
| | | | | family 3, member B (CLEC3B), mRNA | | |
| IPI00007778 | F6X5H7\|B2RBF5\|Q5VX51\|Q | 1486 | CTBS | cDNA PSEC0114fis, clone | 25.8 | 0.0045 |
| | 5VX50\|Q8TC97\|B3KQS3\|B4 | | | NT2RP2006543, highly | | |
| | DQ98\|Q01459 | | | similar to DI-N- | | |
| | | | | ACETYLCHITOBIASE (EC 3.2.1.-)\| | | |
| | | | | CTBS protein\|Di-N- | | |
| | | | | acetylchitobiase\|cDNA FLJ55135, | | |
| | | | | highly similar to Di-N- | | |
| | | | | acetylchitobiase (EC 3.2.1.-)\| | | |
| | | | | cDNA, FLJ95483, highly similar to Homo | | |
| | | | | sapiens chitobiase, di-N-acetyl-(CTBS), | | |
| | | | | mRNA\|Chitobiase, di-N-acetyl- | | |
| IPI00006662 | D3DNW6\|B2R579\|P05090\|Q6 | 347 | APOD | Apolipoprotein D | 25.6 | 0.0239 |
| | IBG6 | | | | | |
| IPI00299738 | O14550\|A4D2D2\|B2R9E1\|Q1 | 5118 | PCOLCE | Procollagen C-endopeptidase enhancer | 23.9 | 0.0214 |
| | 5113 | | | Procollagen C-endopeptidase enhancer 1 | | |
| IPI00027843 | P22891\|A6NMB4\|Q5JVF6\|Q1 | 8858 | PROZ | Vitamin K-dependent protein Z | 23.0 | 0.0009 |
| | 5213\|Q5JVF5 | | | | | |
| IPI00021085 | O75594\|Q4VB36 | 8993 | PGLYRP1 | Peptidoglycan recognition protein 1 | 21.4 | 0.0262 |
| IPI00009030 | P13473\|Q16641\|D3DTF0\|Q6Q | 3920 | LAMP2 | Lysosome-associated | 21.2 | 0.0235 |
| | 3G8\|Q99534\|A8K4X5\|Q9UD9 | | | membrane glycoprotein 2 | | |
| | 3\|Q96J30 | | | | | |
| IPI00395488 | Q6UXL4\|Q6UXL5\|Q96CX1\| | 114990 | VASN | Vasorin | 21.2 | 0.0017 |
| | Q6EMK4 | | | | | |
| IPI00018953 | Q53TN1\|P27487 | 1803 | DPP4 | Dipeptidyl peptidase 4 | 20.3 | 0.0153 |
| IPI00302944 | Q5VYK2\|Q71UR3\|Q5VYK1\| | 1303 | COL12A1 | Collagen alpha-1(XII) chain | 19.6 | 0.0256 |
| | Q15955\|Q99716\|Q99715\|O43 | | | | | |
| | 853 | | | | | |
| IPI00293539 | A8MZC8\|Q9UQ94\|B7WP28\| | 1009 | CDH11 | Cadherin-11 | 19.4 | 0.0246 |
| | Q9UQ93\|A8K5D6\|Q15065\|P5 | | | | | |
| | 5287\|Q15066 | | | | | |
| IPI00027235 | Q9UC75\|Q9NTQ3\|O95414\|O7 | 8455 | ATRN | Uncharacterized protein\|Attractin | 19.3 | 0.0188 |
| | 5882\|Q9UDF5\|Q9NU01\|A8K | | | | | |
| | AE5\|Q9NZ58\|O60295\|Q3MIT | | | | | |
| | 3\|Q9NZ57\|Q5VYW3\|C9IZD4\| | | | | | |
| | Q5TDA4\|Q5TDA2\|Q9NTQ4 | | | | | |
| IPI00026314 | A8MUD1\|B7Z9A0\|P06396\|Q8 | 2934 | GSN | Gelsolin (Amyloidosis, Finnish type)\|cDNA | 19.0 | 0.0436 |
| | WVV7\|B7Z373\|Q5T0I2\|B7Z6 | | | FLJ56154, highly similar to Gelsolin\|cDNA | | |
| | N2 | | | FLJ56212, highly similar to Gelsolin\|Gelsolin | | |
| IPI00216780 | Q6NV88\|Q8IUL8\|Q8WV21\|Q | 148113 | CILP2 | cDNA, FLJ94946, highly similar to | 18.7 | 0.0026 |
| | 8N4A6\|B2RAJ0 | | | Homo sapiens cartilage intermediate | | |
| | | | | layer protein 2 (CILP2), | | |
| | | | | mRNA\|Cartilage intermediate layer protein 2 | | |
| IPI00021885 | Q9BX62\|A8K3E4\|Q4QQH7\|D | 2243 | FGA | cDNA FLJ78367, highly similar to | 18.5 | 0.0163 |
| | 3DP14\|P02671\|D3DP15\|Q9U | | | Homo sapiens fibrinogen, A | | |
| | CH2 | | | alpha polypeptide (FGA), | | |
| | | | | transcriptvariant alpha, mRNA\|Fibrinogen | | |
| | | | | alphachain | | |
| IPI00012585 | P07686 | 3074 | HEXB | Beta-hexosaminidase subunit beta | 18.5 | 0.0494 |
| IPI00060800 | Q96DA0\|C3PTT6\|B2R4F6\|A6 | 124220 | PAUF\|ZG16B | Zymogen granule protein 16 homolog B\| | 17.5 | 0.0227 |
| | NIY1\|Q6UW28 | | | Pancreatic adenocarcinoma upregulated factor | | |
| IPI00176427 | B2R7L5\|Q9Y4A4\|Q8NFZ8 | 199731 | CADM4 | Cell adhesion molecule 4 | 17.3 | 0.0021 |
| IPI00022661 | Q92692\|Q96J29\|Q6IBI6\|O754 | 5819 | PVRL2 | Poliovirus receptor-related protein | 16.7 | 0.0454 |
| | 55\|Q7Z456 | | | 2\|Poliovirus receptor related 2 | | |
| IPI00291262 | Q5HYC1\|Q2TU75\|B3KSE6\|Q | 1191 | CLU | Clusterin | 16.2 | 0.0096 |
| | 7Z5B9\|B2R9Q1\|P11381\|P113 | | | | | |
| | 80\|P10909 | | | | | |
| IPI00221224 | Q6GT90\|Q8IVL7\|B4DP01\|Q5 | 290 | ANPEP\|CD13 | cDNA FLJ56158, highly similar to | 16.1 | 0.0111 |
| | 9E93\|Q16728\|Q8IUK3\|Q8IVH | | | Aminopeptidase N (EC 3.4.11.2)\|Membrane | | |
| | 3\|P15144\|Q71E46\|B4DV63\|B | | | alanine aminopeptidase | | |
| | 4DPH5\|B4DP96\|Q9UCE0 | | | variant\|Uncharacterized protein\| | | |
| | | | | Aminopeptidase N\|cDNA FLJ56120, highly | | |
| | | | | similar to Aminopeptidase N (EC | | |
| | | | | 3.4.11.2)\|cDNA FLJ55496, highly similar to | | |
| | | | | Aminopeptidase N (EC 3.4.11.2) | | |
| IPI00291867 | Q6LAM0\|P05156\|O60442 | 3426 | CFI | Complement factor I\|Light chain of factor I | 15.0 | 0.0147 |
| IPI00003919 | Q16770\|Q3KRG6\|Q16769\|Q5 | 25797 | tmp_locus_46\| | Glutaminyl-peptide cyclotransferase\| | 14.3 | 0.0121 |
| | 3TR4 | | QPCT | Glutaminyl-peptide cyclotransferase | | |
| | | | | (Glutaminyl cyclase), isoform CRA_a | | |
| IPI00099670 | P19835\|Q9UP41\|Q16398\|O75 | 1056 | CEL | cDNA FLJ51297, highly similar to Bile | 13.8 | 0.0464 |
| | 612\|B4DSX9\|Q9UCH1\|Q5T7 | | | salt-activated lipase (EC 3.1.1.3)\|Bile | | |
| | U7 | | | salt-dependent lipase oncofetal | | |
| | | | | isoformlBile salt-activated lipase | | |
| IPI00031065 | Q14UV0\|Q14UU9\|P24855 | 1773 | DNASE1 | Deoxyribonuclease\|Deoxyribonuclease-1 | 13.8 | 0.0044 |
| IPI00043992 | Q96K15\|Q96NY8 | 81607 | PVRL4 | Poliovirus receptor-related protein 4 | 13.7 | 0.0332 |
| IPI00015525 | Q504V7\|B4E3H8\|Q6P2N2\|Q9 | 79812 | MMRN2 | Multimerin-2\|cDNA FLJ54082, | 13.7 | 0.0046 |
| | H8L6 | | | highly similar to Multimerin-2 | | |
| IPI00009027 | Q21BE1\|P05451\|Q0VFX1\|A8 | 5967 | REG1A | REG1A protein\|putative | 13.6 | 0.0282 |
| | K7G6\|P11379\|Q4ZG28 | | | uncharacterized protein | | |
| | | | | REG1A\|cDNAFLJ75763, highly similar to | | |
| | | | | Homo sapiens regenerating islet-derived | | |
| | | | | 1 alpha (pancreatic stone protein, | | |
| | | | | pancreatic thread protein) (REG1A), | | |
| | | | | mRNA\|Lithostathine-1-alpha | | |
| IPI00022432 | Q9UBZ6\|Q6IB96\|P02766\|E9K | 7276 | TTR | Epididymis tissue sperm binding protein Li | 13.3 | 0.0042 |
| | L36\|Q549C7\|Q9UCM9 | | | 4a\|Transthyretin | | |
| IPI00022290 | P60022\|Q09753\|Q86SQ8 | 1672 | DEFB1\|HBD1 | Beta-defensin-1\|Beta-defensin 1 | 13.3 | 0.0053 |
| IPI00022420 | D3DR38\|P02753\|Q9P178\|Q8 | 5950 | RBP4 | Retinol-binding protein 4 | 13.2 | 0.0087 |
| | WWA3\|Q5VY24\|O43479\|O43 | | | | | |
| | 478 | | | | | |
| IPI00102300 | Q9UIF2\|Q9HCN7\|Q9HCN6 | 51206 | GP6 | Platelet glycoprotein VI | 13.1 | 0.0032 |
| IPI00240345 | Q695G9\|Q86T13\|Q6PWT6\|Q8 | 161198 | CLEC14A | C-type lectin domain family 14 member A | 12.9 | 0.0015 |
| | N5V5 | | | | | |
| IPI00153049 | Q5TA39\|Q96KC3\|Q9BRK3 | 54587 | MXRA8 | Matrix-remodeling-associated protein 8 | 12.9 | 0.0286 |
| IPI00029658 | A8KAJ3\|Q541U7\|Q12805\|A8 | 2202 | EFEMP1 | EGF-containing fibulin-like extracellular | 12.9 | 0.0256 |
| | K3I4\|D6W5D2\|Q59G97\|B2R6 | | | matrix protein 1 isoform b variant\|EGF- | | |
| | M6 | | | containing fibulin-like extracellular | | |
| | | | | matrix protein 1\|cDNA, FLJ93024, highly | | |
| | | | | similar to Homo sapiens EGF- | | |
| | | | | containing fibulin-like | | |
| | | | | extracellular matrix protein | | |
| | | | | 1 (EFEMP1), transcript | | |
| | | | | variant 1, mRNA\|cDNA FLJ77823, | | |
| | | | | highly similar to | | |
| | | | | Homo sapiens EGF-containing | | |
| | | | | fibulin-like extracellular | | |
| | | | | matrix protein 1, transcript variant 3, mRNA | | |
| IPI00219684 | Q5VV93\|B2RAB6\|Q99957\|P0 | 2170 | FABP3 | FABP3 protein\|fatty acid- | 12.8 | 0.0009 |
| | 5413\|Q6IBD7 | | | binding protein, heart | | |
| IPI00302592 | Q5HY55\|Q5HY53\|P21333\|Q8 | 2316 | FLNA\|FLJ00119 | Filamin-A\|Filamin A\|FLNA protein\| | 12.8 | 0.0025 |
| | NF52\|Q60FE6\|Q6NXF2\|Q81E | | | FLJ00119 protein | | |
| | S4 | | | | | |
| IPI00019568 | P00734\|B4DDT3\|B2R7F7\|Q5 | 2147 | F2 | Prothrombin B-chain\|cDNA | 12.1 | 0.0383 |
| | 3H06\|Q53H04\|Q9UCA1\|Q69E | | | FLJ54622, highly similar to | | |
| | Z8\|Q4QZ40\|Q7Z7P3\|B4E1A7\| | | | Prothrombin (EC 3.4.21.5)\|Prothrombin | | |
| | Q69EZ7 | | | | | |
| IPI00075248 | Q96HK3\|P02593\|P70667\|Q13 | 801\|808\|805 | CALM2\|CALM3\| | Calmodulin\|Calmodulin 1 | 12.1 | 0.0234 |
| | 942\|P99014\|P62158\|B4DJ51\|Q | | CALM1 | (Phosphorylase kinase, delta), | | |
| | 53S29\|Q61379\|Q61380 | | | isoform CRA_a | | |
| IPI00103871 | Q9NWJ8\|A8K154\|Q8TEG1\|Q | 54538 | ROBO4 | Roundabout homolog 4 | 11.9 | 0.0291 |
| | 8WZ75\|Q96JV6\|Q9H718\|Q14 | | | | | |
| | DU7 | | | | | |
| IPI00009793 | Q53GX9\|Q9NZP8 | 51279 | C1RL | Complement C1r subcomponent- | 11.7 | 0.0142 |
| IPI00299086 | O00173\|O43391\|O00560\|B2R | 6386 | SDCBP | like protein | 11.7 | 0.0132 |
| | 5Q7\|B4DUH3\|Q14CP2\|B7ZL | | | Syntenin-1\|Syndecan binding | | |
| | N2 | | | protein (Syntenin) | | |
| IPI00019157 | D3DW77\|Q92675\|Q6UVK1 | 1464 | CSPG4 | Chondroitin sulfate proteoglycan 4 | 11.7 | 0.0185 |
| IPI00006971 | Q2M2V5\|Q9HCU0\|Q96KB6\| | 57124 | CD248 | Endosialin | 11.3 | 0.0186 |
| | Q3SX55 | | | | | |
| IPI00555812 | Q53F31\|P02774\|B4DPP2\|Q16 | 2638 | GC | Vitamin D-binding protein | 11.3 | 0.0073 |
| | 309\|Q16310\|Q6GTG1 | | | | | |
| IPI00009276 | Q14218\|Q9ULX1\|Q96CB3\|B2 | 10544 | PROCR | Endothelial protein C receptor | 10.9 | 0.0332 |
| | RC04\|Q9UNN8\|Q6IB56 | | | | | |
| IPI00013955 | Q9UE76\|Q9UE75\|Q9UQL1\|Q | 4582 | MUC1 | Mucin-1 | 10.9 | 0.0144 |
| | 7Z552\|Q14876\|Q9Y4J2\|Q141 | | | | | |
| | 28\|Q16437\|P13931\|P17626\|P1 | | | | | |
| | 5941\|Q16615\|P15942\|Q16442\| | | | | | |
| | Q9BXA4 | | | | | |
| IPI00010343 | Q9UPR5\|B4DYQ9\|B4DEZ4 | 6543 | SLC8A2 | cDNA FLJ58526, highly | 10.7 | 0.0069 |
| | | | | similar to Sodium/calcium | | |
| | | | | exchanger 2\|Sodium/calcium exchanger 2 | | |
| IPI00011302 | P13987\|Q6FHM9 | 966 | CD59 | CD59 antigen, complement regulatory | 10.1 | 0.0171 |
| | | | | protein, isoform CRA_b\|CD59 glycoprotein | | |
| IPI00017601 | Q2PP18\|A8K5A4\|Q1L857\|A5 | 1356 | CP | cDNA FLJ76826, highly similar | 9.7 | 0.0247 |
| | | | | to Homo sapiens | | |
| | PL27\|B3KTA8\|Q14063\|P0045 | | | ceruloplasmin (ferroxidase) (CP), | | |
| | 0\|Q9UKS4 | | | mRNA\|cDNA FLJ37971 fis, clone | | |
| | | | | CT0NG2009958, highly similar to | | |
| | | | | CERULOPLASMIN(EC 1.16.3.1)\|CP | | |
| | | | | protein\|Ceruloplasmin | | |
| IPI00553177 | E9KL23\|Q0PVP5\|Q53XB8\|Q9 | 5265 | SERPINA1 | Epididymis secretory | 9.6 | 0.0265 |
| | 6BF9\|B2RDQ8\|Q13672\|Q5U0 | | | sperm binding protein Li | | |
| | M1\|Q7M4R2\|P01009\|Q9P1P0\| | | | 44a\|Alpha-1-antitrypsin | | |
| | Q9UCM3\|A6PX14\|Q9UCE6\| | | | | | |
| | Q96ES1\|Q86U191Q86U18 | | | | | |
| IPI00032293 | D3DW42\|B2R5J9\|P01034\|E9 | 1471 | CST3 | Cystatin-C\|Cystatin C | 9.2 | 0.0021 |
| | RH26\|Q6FGW9 | | | | | |
| IPI00045512 | Q69YJ3\|Q5TYR7\|Q96RW7\|Q | 83872 | DKFZp762L185\|H | Hemicentin 1\|cDNA FLJ14438 fis, clone | 9.0 | 0.0171 |
| | 96DN8\|Q96SC3\|Q5TCP6\|Q96 | | MCN1 | HEMBB1000317, weakly similar to | | |
| | DN3\|Q96K89\|A6NGE3 | | | FIBULIN-1, ISOFORM D\| | | |
| | | | | Putative uncharacterized | | |
| | | | | protein DKFZp762L185\|Hemicentin-1 | | |
| IPI00010675 | Q15854\|Q03403 | 7032 | TFF2 | Trefoil factor 2 | 8.9 | 0.0247 |
| IPI00032325 | P01040\|Q6IB90 | 1475 | CSTA | CSTA protein\|Cystatin-A | 8.7 | 0.0042 |
| IPI00298388 | Q49A94\|Q8NCJ9\|Q96FE7\|Q8 | 113791 | PIK3IPI | Phosphoinositide-3-kinase- | 8.2 | 0.0075 |
| | 6YW2\|O00318 | | | interacting protein 1 | | |
| IPI00306322 | Q14052\|Q548C3\|Q66K23\|P08 | 1284 | COL4A2 | cDNA FLJ56433, highly similar to Collagen | 7.5 | 0.0264 |
| | 572\|Q5VZA9\|B4DH43 | | | alpha-2(IV) chain\|Collagen alpha-2(IV) chain | | |
| IPI00290085 | Q14923\|Q8N173\|B0YIY6\|P19 | 1000 | CDH2 | Cadherin-2 | 7.1 | 0.0137 |
| | 022 | | | | | |
| IPI00010949 | Q9HAT2\|B3KPB0\|Q9HAU7\| | 54414 | SIAE | Sialate O-acetylesterase | 7.1 | 0.0060 |
| | Q8IUT9\|Q9NT71 | | | | | |
| IPI00295414 | P39059\|B3KTP7\|Q5T6J4\|Q9Y | 1306 | COL15A1 | Collagen alpha-1(XV) chainlcDNA | 6.8 | 0.0135 |
| | 4W4\|Q9UDC5 | | | FLJ38566 fis, clone HCHON2005118,highly | | |
| | | | | similar to Collagen alpha-1(XV) chain | | |
| IPI00010182 | P08869\|Q4VWZ6\|Q53SQ7\|Q9 | 1622 | DBI | Diazepam binding inhibitor, | 6.8 | 0.0021 |
| | UCI8\|P07108\|B8ZWD8\|Q6IB | | | splice form 1D(1)Acyl- | | |
| | 48 | | | CoA-binding protein | | |
| IPI00103636 | Q8WXW1\|Q6IB27\|A6PVD5\| | 10406 | WFDC2 | WAP four-disulfide core domain protein 2 | 6.6 | 0.0191 |
| | Q96KJ1\|A2A2A5\|Q14508\|Q8 | | | | | |
| | WXV9\|A2A2A6\|Q8WXW0\|Q | | | | | |
| | 8WXW2 | | | | | |
| IPI00289983 | Q96QM0\|D3DNC6\|Q96KY0\| | 55 | ACPP | Prostatic acid phosphatase | 6.5 | 0.0073 |
| | P15309\|Q96QK9 | | | | | |
| IPI00027482 | B2R9F2\|P08185\|Q7Z2Q9\|A8 | 866 | SERPINA6 | Corticosteroid-binding globulin1cDNA, | 6.5 | 0.0256 |
| | K456 | | | FLJ94361, highly similar to | | |
| | | | | Homo sapiens serine | | |
| | | | | (or cysteine) proteinase inhibitor, clade | | |
| | | | | A(alpha-1 antiproteinase, antitrypsin), | | |
| | | | | member 6 (SERPINA6), mRNA | | |
| IPI00175092 | Q53SV6\|Q8WUU3\|Q8NC42\| | 284996 | RNF149\| | Putative uncharacterized protein | 6.4 | 0.0102 |
| | | | | LOC284996\|E3 | | |
| | Q8NBY5\|Q53S14\|Q8N5I8 | | LOC284996 | ubiquitin-protein ligase RNF149 | | |
| IPI00186826 | B5A9721B5A9701Q96L35 | 2050 | EPHB4 | EPH receptor B4, isoform CRA_b\|Soluble | 6.1 | 0.0396 |
| | | | | EPHB4 variant 1\|Soluble EPHB4 variant 3 | | |
| IPI00019580 | B2R7F8\|P00747\|Q9UMI2\|Q15 | 5340 | PLG | PLG protein\|Plasminogen\|cDNA, | 6.1 | 0.0084 |
| | 146\|Q5TEH4\|Q6PA00\|B4DPH | | | FLJ93426, highly similar to Homo | | |
| | 4 | | | sapiens plasminogen (PLG), | | |
| | | | | mRNA\|cDNAFLJ58778, | | |
| | | | | highly similar to Plasminogen | | |
| | | | | (EC 3.4.21.7) | | |
| IPI00032258 | B0QZR6\|Q13160\|A7E2V2\|Q1 | 7201721 | C4A variant | Complement C4-A\|C4A | 6.0 | 0.0480 |
| | 4033\|P0C0L4\|B7ZVZ6\|Q6P4 | | protein\|C4A | variant protein\|Complement | | |
| | R1\|B2RUT6\|Q5JQM8\|Q4LE8 | | | component 4A (Rodgers blood group) | | |
| | 2\|P01028\|Q9NPK5\|P78445\|Q1 | | | | | |
| | 3906\|Q14835\|Q9UIP5 | | | | | |
| IPI00292130 | A8K981\|Q9UIX8\|Q07507\|Q8 | 1805 | DPT | Dermatopontin | 5.9 | 0.0022 |
| | N4R2 | | | | | |
| IPI00029275 | P08582\|Q9BQE2 | 4241 | MFI2 | Melanotransferrin | 5.8 | 0.0252 |
| IPI00019906 | B4DY23\|P35613\|Q7Z796\|Q54 | 682 | hEMMPRIN\|BSG | Basigin\|cDNA FLJ61188, highly similar to | 5.7 | 0.0082 |
| | A51\|Q8IZL7 | | | Basigin\|Basigin (Ok blood group), | | |
| | | | | isoform CRA_a | | |
| IPI00218413 | Q96EM9\|B7Z7C9\|B2R865\|P4 | 686 | BTD | Biotinidase\|cDNA FLJ50907, highly similar to | 5.6 | 0.0416 |
| | 3251 | | | Biotinidase (EC 3.5.1.12) | | |
| IPI00026926 | Q02747 | 2980 | GUCA2A | Guanylin | 5.5 | 0.0152 |
| IPI00025992 | B6EU04\|Q9BY68\|Q1HE14\|P8 | 57817 | HAMP | Hepcidin\|Hepcidin antimicrobial peptide | 5.5 | 0.0484 |
| | 1172 | | | | | |
| IPI00179330 | B2RDW1\|Q9UEK8\|Q8WYN8 | 6233 | RPS27A | Ribosomal protein S27a\|Ubiquitin- | 5.2 | 0.0004 |
| | \|Q91887\|Q6LDU5\|P62988\|Q9 | | | 40S ribosomal protein | | |
| | BX98\|Q9UEF2\|P62979\|Q5RK | | | S27a\|Ribosomal protein S27a, isoform CRA_c | | |
| | T7\|Q9UPK7\|P14798\|Q9BWD | | | | | |
| | 6\|Q6LBL4\|P02248\|P02249\|Q9 | | | | | |
| | 1888\|Q9BQ77\|Q29120\|P0225 | | | | | |
| | 0\|Q9UEG1 | | | | | |
| IPI00099110 | Q9Y4V9\|B1ARE9\|B1ARE8\|Q | 1755 | DMBT1 | Deleted in malignant brain tumors 1 protein | 5.0 | 0.0038 |
| | 5JR26\|B1ARF0\|Q9UGM3\|Q9 | | | | | |
| | UGM2\|Q59EX0\|B1ARE7\|A8 | | | | | |
| | E4R5\|Q9UKJ4\|Q9UJ57\|Q96D | | | | | |
| | U4\|A6NDG4\|Q9Y211\|Q6MZ | | | | | |
| | N4\|A6NDJ5 | | | | | |
| IPI00291488 | Q8WXW1\|Q6IB27\|A6PVD5\| | 10406 | WFDC2 | WAP four-disulfide core domain protein 2 | 5.0 | 0.0413 |
| | Q96KJ1\|A2A2A5\|Q14508\|Q8 | | | | | |
| | WXV9\|A2A2A6\|Q8WXW0\|Q | | | | | |
| | 8WXW2 | | | | | |
| IPI00002435 | P26842\|B2RDZ0 | 939 | CD27 | CD27 antigen | 5.0 | 0.0003 |
| IPI00021447 | B3KXB7\|D3DT76\|P19961\|Q9 | 280 | AMY2B | Alpha-amylase 2B | 4.9 | 0.0477 |
| | UBH3 | | | | | |
| IPI00303161 | Q96AP7\|Q96T50 | 90952 | ESAM | Endothelial cell-selective adhesion molecule | 4.8 | 0.0008 |
| IPI00000024 | B4E2D8\|Q8IUP2\|Q08174 | 5097 | PCDH1 | cDNA FLJ59655, highly similar to | 4.6 | 0.0079 |
| | | | | Protocadherin-1\|Protocadherin-1 | | |
| IPI00002280 | Q9UHG2\|Q4VC04 | 27344 | PCSK1N | ProSAAS | 4.5 | 0.0007 |
| IPI00009650 | Q5T8A1\|P31025 | 3933 | LCN1 | Lipocalin-1 | 4.4 | 0.0053 |
| IPI00021841 | Q9UCS8\|Q6LDN9\|Q9UCT8\| | 335 | APOA1 | APOA1 proteinApolipoprotein A-I | 4.4 | 0.0233 |
| | A8K866\|P02647\|Q6Q785\|Q6L | | | | | |
| | EJ8 | | | | | |
| IPI00977659 | Q6S9E4\|A8K9Q3\|Q14C97\|Q9 | 8322 | GPCR\|FZD4 | Frizzled-4\|Putative G-protein coupled receptor | 4.2 | 0.0057 |
| | ULV1\|Q8TDT8 | | | | | |
| IPI00219365 | Q6PJT4\|P26038 | 4478 | MSN | MSN protein\|Moesin | 4.1 | 0.0033 |
| IPI00289334 | Q9UEV9\|Q13706\|Q9NT26\|C9 | 2317 | FLNB | Filamin-B | 4.1 | 0.0268 |
| | JMC4\|Q6MZJ1\|C9JKE6\|O753 | | | | | |
| | 69\|Q8WXS9\|B2ZZ84\|B2ZZ85 | | | | | |
| | \|Q8WXT1\|Q8WXT0\|Q59EC2\| | | | | | |
| | Q8WXT2\|Q9NRB5 | | | | | |
| IPI00216298 | P10599\|Q53X69\|Q9UDG5\|Q9 | 7295 | TXN | Thioredoxin | 4.0 | 0.0028 |
| | 6KI3 | | | | | |
| IPI00013576 | Q8WVV5\|O00480 | 10385 | BTN2A2 | Butyrophilin subfamily 2 member A2 | 4.0 | 0.0141 |
| IPI00376457 | B4E0V9 | 342510 | | cDNA FLJ61198, highly similar | 4.0 | 0.0064 |
| | | | | to Homo sapiens CD300 antigen | | |
| | | | | like family member E (CD300LE), mRNA | | |
| IPI00296992 | Q8N5L2\|P30530\|Q9UD27 | 558 | AXL | Tyrosine-protein kinase receptor UFO | 3.9 | 0.0454 |
| IPI00022284 | Q15216\|A1YVW6\|Q8TBG0\|Q | 5621 | PRNP | Major prion protein | 3.8 | 0.0118 |
| | 27H91\|P04156\|Q86XR1\|O604 | | | | | |
| | 89\|Q5QPB4\|Q6FGR8\|Q15221\| | | | | | |
| | Q6FGN5\|D4P3Q7\|Q96E70\|P7 | | | | | |
| | 8446\|B4DDS1\|Q9UP19\|B2R5 | | | | | |
| | Q9\|Q5U0K3\|Q540C4\|Q53YK | | | | | |
| | 7 | | | | | |
| IPI00289501 | O15240\|Q9UDW8 | 7425 | VGF | Neurosecretory protein VGF | 3.8 | 0.0102 |
| IPI00001754 | Q9Y624\|D3DVF0\|Q6FIB4 | 50848 | F11R | F11 receptor\|FLJ receptor, isoform CRA_ | 3.6 | 0.0048 |
| | | | | a\|Junctional adhesion molecule A | | |
| IPI00027463 | P06703\|Q5RHS4\|D3DV39\|B2 | 6277 | S100A6 | cDNA, FLJ92369, highly similar to | 3.6 | 0.0207 |
| | R577 | | | Homo sapiens S100 calcium | | |
| | | | | binding protein A6 (calcyclin) | | |
| | | | | (S100A6),mRNA\|Protein S100-A6 | | |
| IPI00297646 | O76045\|Q16050\|Q9UML6\|Q1 | 1277 | COL1A1 | Collagen type I alpha 1\|Type II procollagen | 3.6 | 0.0160 |
| | 3902\|Q14037\|Q13903\|Q8IVI5\| | | | gene\|Collagen, type I, alpha 1, | | |
| | Q6LAN8\|P02452\|Q13896\|Q59 | | | isoform CRA_a\|Type I collagen alpha 1 | | |
| | F64\|Q15176\|D3DTX7\|Q8N47 | | | chain\|Collagen alpha-1(I) chain | | |
| | 3\|Q15201\|Q14042\|Q14992\|Q9 | | | | | |
| | UMM7\|Q7KZ30\|P78441\|Q7K | | | | | |
| | Z34\|Q9UMA6 | | | | | |
| IPI00025204 | A8K7M5\|O43866\|Q6UX63 | 922 | CD5L | CD5 antigen-like | 3.6 | 0.0014 |
| IPI00470360 | Q8TB15\|Q5XKC6\|Q9H9N\|Q | 55243 | KIRREL | Kin of IRRE-like protein 1 | 3.5 | 0.0062 |
| | 7Z7N8\|Q5W0F8\|Q96J84\|Q9N | | | | | |
| | VA5\|Q7Z696 | | | | | |
| IPI00002910 | Q9H665\|Q8N5X0 | 79713 | IGFLR1 | IGF-like family receptor 1 | 3.5 | 0.0090 |
| IPI00641251 | B2RDS5\|Q53HF7\|Q9NPF0\|D | 51293 | CD320 | CD320 antigen | 3.3 | 0.0078 |
| | 6W668 | | | | | |
| IPI00027509 | B7Z747\|Q9UCJ9\|B7Z8A9\|P14 | 4318 | MMP9 | cDNA FLJ51036, highly similar to Matrix | 3.3 | 0.0218 |
| | 780\|Q8N725\|Q9UDK2\|Q3LR7 | | | metalloproteinase-9 (EC3.4.24.35)\| | | |
| | 0\|Q9UCL1\|F5GY52\|Q9H4Z1\| | | | Uncharacterized protein\| | | |
| | B2R7V9\|Q9Y354\|B7Z507 | | | Matrix metalloproteinase-9\|Matrix | | |
| | | | | metalloproteinase 9\|cDNA FLJ51120, | | |
| | | | | highly similar to Matrix | | |
| | | | | metalloproteinase-9 (EC 3.4.24.35)\|cDNA | | |
| | | | | FLJ51166, highly similar to Matrix | | |
| | | | | metalloproteinase-9 (EC 3.4.24.35) | | |
| IPI00021968 | Q9Y6Q6 | 8792 | TNFRSF11A | Tumor necrosis factor | 3.2 | 0.0112 |
| | | | | receptor superfamily member 11A | | |
| IPI00027436 | B2R961\|P08138 | 4804 | NGFR | Tumor necrosis factor receptor | 3.2 | 0.0117 |
| | | | | superfamily member 16 | | |
| IPI00003813 | Q9BY67\|Q8N2F4\|Q86WB8\|Q | 23705 | DKFZp686F1789\| | Putative uncharacterized protein | 3.1 | 0.0197 |
| | 6MZK6 | | CADM1 | DKFZp686F1789\|Cell adhesion molecule 1 | | |
| IPI00006705 | P11684\|Q9UCM4\|B2R5F2\|Q6 | 7356 | SCGB1A1 | Uteroglobin | 3.1 | 0.0305 |
| | FHH3\|Q9UCM2 | | | | | |
| IPI00013972 | Q16574\|Q0Z7S6\|O60399\|P31 | 1088 | CEACAM8 | Carcinoembryonic antigen-related cell | 3.1 | 0.0046 |
| | 997 | | | adhesion molecule 8 | | |
| IPI00289831 | Q16341\|O75255\|Q15718\|Q13 | 5802 | PTPRS | Receptor-type tyrosine-protein phosphatase | 3.0 | 0.0328 |
| | 332\|O75870\|D6W633\|Q2M3R | | | S\|Protein tyrosine phosphatase, receptor | | |
| | 7 | | | type, S, isoform CRA_a | | |
| IPI00003101 | P01589\|B2R9M9\|A2N4P8\|Q5 | 3559 | IL2RA\|IL2R | cDNA, FLJ94475, highly similar to Homo | 3.0 | 0.0085 |
| | W007\|Q53FH4 | | | sapiens interleukin 2 receptor, alpha | | |
| | | | | (IL2RA), mRNA\|IL2R | | |
| | | | | protein\|Interleukin-2 receptor subunit | | |
| | | | | alpha\|Interleukin 2 receptor, alpha | | |
| | | | | chain variant | | |
| IPI00017202 | Q7Z798\|Q7Z7A0\|Q7Z799\|Q9 | 140578 | CHODL | Chondrolectin | 3.0 | 0.0341 |
| | H9P2\|B2R9C0\|Q9HCY3 | | | | | |
| IPI00031121 | B3KXD3\|B3KR42\|P16870\|D3 | 1363 | CPE | cDNA FLJ45230 fis, clone BRCAN2021325, | 3.0 | 0.0327 |
| | DP33\|A8K4N1\|Q9UIU9 | | | highly similar to Carboxypeptidase E (EC | | |
| | | | | 3.4.17.10)\|Carboxypeptidase E | | |
| IPI00010290 | Q6FGL7\|Q05CP7\|P07148 | 2168 | FABP1 | Fatty acid-binding protein, liver\|FABP1 protein | 2.9 | 0.0039 |
| IPI00018434 | Q9BUM5\|Q99816 | 7251 | TSG101 | Tumor susceptibility gene 101 protein | 2.8 | 0.0173 |
| IPI00219465 | Q9UDM0\|Q9BVI8\|P20062\|Q9 | 6948 | TCN2 | Transcobalamin-2 | 2.8 | 0.0339 |
| | UCI6\|Q9UCI5 | | | | | |
| IPI00009794 | B1AME5\|B1AME6\|Q8NBQ3\| | 51150 | SDF4 | 45 kDa calcium-binding protein | 2.8 | 0.0403 |
| | Q96AA1\|Q53HQ9\|B4DSM1\|B | | | | | |
| | 2RDF1\|Q9BRK5\|Q9NZP7\|Q9 | | | | | |
| | UN53\|Q53G52 | | | | | |
| IPI00219860 | P23468\|B1ALA0 | 5789 | PTPRD | Receptor-type tyrosine-protein | 2.8 | 0.0437 |
| | | | | phosphatase delta | | |
| IPI00329538 | Q9UCA3\|Q16651 | 5652 | PRSS8 | Prostasin | 2.7 | 0.0164 |
| IPI00166729 | O60386\|Q5XKQ4\|P25311\|D6 | 563 | AZGP1 | Zinc-alpha-2-glycoprotein | 2.6 | 0.0168 |
| | W5T8\|Q8N4N0 | | | | | |
| IPI00016786 | P25763\|P21181\|P60953\|Q9UD | 998 | CDC42 | Cell division control protein 42 homolog | 2.6 | 0.0011 |
| | I2\|Q7L8R5 | | | | | |
| IPI00215997 | Q96ES4\|P21926\|Q5J7W6\|D3 | 928 | CD9 | CD9 antigen | 2.6 | 0.0200 |
| | DUQ9 | | | | | |
| IPI00383032 | Q96K94\|B2RAY2\|Q8WW60\| | 84868 | HAVCR2 | Hepatitis A virus cellular receptor 2 | 2.6 | 0.0202 |
| | Q8TDQ0 | | | | | |
| IPI00010807 | Q9H461 | 8325 | FZD8 | Frizzled-8 | 2.6 | 0.0030 |
| IPI00034319 | Q9NYQ9\|O60888\|Q5JXM9\|Q | 51596 | CUTA | Protein CutA | 2.5 | 0.0245 |
| | 3B784\|A2BEL4\|A2AB26\|Q5S | | | | | |
| | U05 | | | | | |
| IPI00026154 | B4DJQ5\|P14314\|Q96BU9\|Q9 | 5589 | PRKCSH | Glucosidase 2 subunit beta\|Uncharacterized | 2.5 | 0.0008 |
| | P0W9\|E7EQZ9\|Q96D06 | | | protein\|cDNA FLJ59211, highly similar | | |
| | | | | to Glucosidase 2 subunit beta | | |
| IPI00220737 | Q96CJ3\|Q16180\|B7Z8D6\|Q15 | 4684 | NCAM1 | cDNA FLJ54771, highly similar to Neural | 2.4 | 0.0028 |
| | 829\|Q05C58\|P13591\|P13592\|P | | | cell adhesion molecule 1, 120 | | |
| | 13593\|Q86X47\|Q59FL7\|A8K8 | | | kDa isoform\|Neural cell adhesion | | |
| | T8\|Q16209 | | | molecule 1 | | |
| IPI00925540 | A6NLA3\|Q13350\|Q14870\|P26 | 4485 | MST1 | Hepatocyte growth factor-like protein\|cDNA | 2.4 | 0.0016 |
| | 927\|Q6GTN4\|A8MSX3\|Q53G | | | FLJ56324, highly similar to Hepatocyte | | |
| | N8\|B7Z250 | | | growth factor-like protein\| | | |
| | | | | Macrophage stimulating | | |
| | | | | 1 (Hepatocyte growth factor-like) variant | | |
| IPI00017557 | Q1ZYW2\|Q6PD64\|Q4G124\|Q | 6424 | SFRP4 | Secreted frizzled-related protein 4 | 2.3 | 0.0460 |
| | 6FHJ7\|Q6FHM0\|O14877\|B4D | | | | | |
| | YC1\|Q05BG7 | | | | | |
| IPI00002666 | Q7M4M8\|P09086\|Q16648\|Q9 | 5452 | OCT-2\|POU2F2 | Homeobox protein\|Oct-2 factor\|POU | 2.3 | 0.0004 |
| | BRS4\|Q9UMI6\|Q9UMJ4 | | | domain, class 2, transcription factor 2 | | |
| IPI00414896 | Q9BZ46\|Q9BZ47\|B2RDA7\|E | 8635 | RNASET2 | Ribonuclease T2 | 2.3 | 0.0131 |
| | 1P5C3\|Q8TCU2\|O00584\|Q5T | | | | | |
| | 8Q0 | | | | | |
| IPI00293836 | Q8N3J6\|Q658Q7\|Q8IZP8\|Q3 | 253559 | CADM2 | Cell adhesion molecule 2 | 2.3 | 0.0230 |
| | KQY9 | | | | | |
| IPI00020557 | Q59FG2\|Q07954\|Q6LAF4\|Q2 | 4035 | LRP\|LRP1 | LRP protein\|Alpha-2 macroglobulin | 2.3 | 0.0465 |
| | PP12\|Q8IVG8\|Q6LBN5 | | | receptor\|Prolow-density lipoprotein | | |
| | | | | receptor-related protein 1\|Low | | |
| | | | | density lipoprotein-related protein 1 variant | | |
| IPI00004440 | A8K604\|Q16849\|Q08319\|Q53 | 5798 | PTPRN | cDNA FLJ55332, highly | 2.1 | 0.0139 |
| | QD6\|B4DK12 | | | similar to Receptor-type | | |
| | | | | tyrosine-proteinphosphatase- | | |
| | | | | like N\|Receptor-type | | |
| | | | | tyrosine-protein phosphatase- | | |
| | | | | like N\|cDNA FLJ77469, | | |
| | | | | highly similar to Homo sapiens | | |
| | | | | protein tyrosine | | |
| | | | | phosphatase, receptor type, N, mRNA | | |
| IPI00016450 | Q96TD2\|Q6LCK3\|Q6LCK5\|Q | 6340 | SCNN1G | Amiloride-sensitive sodium channel subunit | 2.1 | 0.0466 |
| | 6LCK4\|Q6LCK6\|Q93023\|A5 | | | gamma\|Amiloride-sensitive epithelial | | |
| | X2V1\|P51170\|Q93026\|Q9302 | | | sodium channel gamma subunit\| | | |
| | 5\|Q93024\|Q93027\|P78437\|Q6 | | | Amiloride-sensitive sodium channel | | |
| | PCC2 | | | gamma-subunit | | |
| IPI00221255 | Q5MY99\|O95797\|O95796\|O9 | 4638 | MYLK | Myosin light chain kinase, smooth muscle | 2.0 | 0.0043 |
| | 5799\|O95798\|Q15746\|Q7Z4J0 | | | | | |
| | \|Q9C0L5\|Q14844\|Q16794\|Q5 | | | | | |
| | MYA0\|Q9UBG5\|Q9UIT9 | | | | | |
| IPI00179185 | O00520\|Q96MX2\|Q66K79 | 8532 | CPZ | Carboxypeptidase Z | 2.0 | 0.0485 |
| IPI00169285 | Q8NHP8 | 196463 | PLBD2 | Putative phospholipase B-like 2 | 1.9 | 0.0040 |
| IPI00152871 | B3KWI4\|Q7RTN7\|Q495Q6\|Q | 131578 | LRRC15 | cDNA FLJ43122 fis, clone CTONG3003737, | 1.9 | 0.0433 |
| | 8TF66 | | | highly similar to Leucine- | | |
| | | | | rich repeat-containing protein | | |
| | | | | 15\|Leucine-rich repeat-containing protein 15 | | |
| IPI00015902 | Q8N5L4\|P09619\|A8KAM8 | 5159 | PDGFRB | cDNA FLJ76012, highly | 1.9 | 0.0161 |
| | | | | similar to Homo sapiens | | |
| | | | | platelet-derived growth factor receptor, | | |
| | | | | betapolypeptide (PDGFRB), mRNA\|Platelet- | | |
| | | | | derived growth factor receptor beta | | |
| IPI00021428 | P02568\|Q5T8M9\|P99020\|P68 | 58 | ACTA1 | Actin, alpha skeletal muscle | 1.9 | 0.0250 |
| | 133 | | | | | |
| IPI00005733 | Q5T7S2\|Q706C0\|P36897\|Q6I | 7046 | TGFBR1 | TGF-beta receptor type-1\|Transforming | 1.9 | 0.0005 |
| | R47\|Q706C1 | | | growth factor beta receptor I | | |
| IPI00030936 | Q5VST0\|D3DQ14\|O60745\|O6 | 10103 | TSPAN1 | Tetraspanin-1 | 1.9 | 0.0306 |
| | 0635 | | | | | |
| IPI00023974 | P53801\|D3DSL9\|A8K274\|Q9 | 754 | PTTG1IP | Pituitary tumor-transforming gene 1 protein- | 1.8 | 0.0070 |
| | NS09\|B2RDP7 | | | interacting protein\|cDNA FLJ78227, | | |
| | | | | highly similar to Homo sapiens | | |
| | | | | pituitary tumor-transforming | | |
| | | | | 1 interacting protein | | |
| | | | | (PTTG1IP),mRNA | | |
| IPI00022830 | Q5JXA5\|Q5JXA4\|B2RD74\|Q | 55968 | NSFL1C | NSFL1 cofactor p47 | 1.7 | 0.0140 |
| | 9UI06\|A2A2L1\|Q9H102\|Q9U | | | | | |
| | NZ2\|Q7Z533\|Q9NVL9 | | | | | |
| IPI00000816 | P42655\|P29360\|Q63631\|Q7M | 7531 | YWHAE | 14-3-3 protein epsilon | 1.7 | 0.0468 |
| | 4R4\|D3DTH5\|Q4VJB6\|Q53X | | | | | |
| | Z5\|P62258\|B3KY71 | | | | | |
| IPI00163563 | Q96S96\|Q8WW74\|Q5EVA1 | 157310 | PEBP4 | Phosphatidylethanolamine-binding protein 4 | 1.6 | 0.0470 |
| IPI00021828 | P04080\|Q76LA1 | 1476 | CSTB | Cystatin-B\|CSTB protein | 1.6 | 0.0027 |
| IPI00029723 | D3DN90\|Q549Z0\|A8K523\|Q1 | 11167 | FSTL1 | cDNA FLJ78447, highly similar to | 1.5 | 0.0075 |
| | 2841 | | | Homo sapiens follistatin-like 1 (FSTL1), | | |
| | | | | mRNA\|Follistatin-related | | |
| | | | | protein 1 | | |
| IPI00183425 | Q8WU72\|QY3F9\|Q9ULV3\| | 25792 | CIZ1 | Cip1-interacting zinc finger | 1.5 | 0.0038 |
| | Q9Y3G0\|Q9UHK4\|A8K9J8\|Q | | | protein\|cDNA FLJ60074, | | |
| | 9H868\|Q5SYW5\|B4E0A3\|Q9 | | | highly similar to Cip1- | | |
| | NYM8\|Q5SYW3 | | | interacting zinc finger protein | | |
| IPI00007257 | O94985\|Q5SR52\|Q5UE58\|Q7 | 22883 | CLSTN1 | Calsyntenin-1 | 1.5 | 0.0118 |
| | 1MN0\|A8K183\|Q8N4K9 | | | | | |

High-risk OSA is associated with a wide variety of related disorders and vulnerabilities, and as such it has a greater need for treatment. High risk OSA is understood to be associated with neurocognitive impairment such as memory impairment, declarative memory defects, learning delays, and issues with academic performance, mood-related disorders such as depression, behavioral issues such as ADHD, aggression, inattentiveness, impulsivity, and excessive sleepiness, cardiovascular risks including hypertension, altherosclerosis, pulmonary hypertension, and left ventricular dysfunction, a metabolic disorders such as dyslipidemia and insulin resistance. Review: Capdevila O S, Kheirandish-Gozal L, Dayyat E, Gozal D. Pediatric obstructive sleep apnea: complications, management, and long-term outcomes. Proc Am Thorac Soc. 2008 Feb. 15; 5(2):274-82. doi: 10.1513/pats.200708-138MG. Review. PubMed PMID: 18250221; PubMed Central PMCID: PMC2645258. Relevant treatments include pharmacological treatment with corticosteroids, leukotriene antagonists, topical nasal steroids, intranasal steroids, and/or montelukast, surgical removal of the adenoids and tonsils, applying positive airway pressure therapy (PAP), or the application of oral applicances. Kheirandish-Gozal L, Bhattacharjee R, Bandla H P, Gozal D. Anti-Inflammatory Therapy Outcomes for Mild OSA in Children. Chest. 2014 Feb. 6. doi: 10.1378/chest.13-2288. [Epub ahead of print] PubMed PMID: 24504096; Marcus C L, Brooks L J, Draper K A, Gozal D, Halbower A C, Jones J, Schechter M S, Ward S D, Sheldon S H, Shiffman R N, Lehmann C, Spruyt K; American Academy of Pediatrics. Diagnosis and management of childhood obstructive sleep apnea syndrome. Pediatrics. 2012 September; 130(3):e714-55. doi: 10.1542/peds.2012-1672. Epub 2012 Aug. 27. Review. PubMed PMID: 22926176.

In certain embodiments, the biomarkers for high-risk OSA are contemplated to constitute the markers identified in Table 2.

TABLE 2
Candidate Biomarkers of High-Risk OSA

| IPI | Gene Symbol | Description |
|---|---|---|
| IPI00014048 | RNASE1 | Ribonuclease pancreatic |
| IPI00302944 | COL12A1 | Isoform 4 of Collagen alpha-1(XII) chain |
| IPI00019449 | RNASE2 | Non-secretory ribonuclease |
| IPI00011302 | CD59 | CD59 glycoprotein |
| IPI00022418 | FN1 | Isoform 1 of Fibronectin |
| IPI00022426 | AMBP | Protein AMBP |
| IPI00328113 | FBN1 | Fibrillin-1 |
| IPI00829813 | PIK3IP1 | Isoform 2 of Phosphoinositide-3-kinase-interacting protein 1 |
| IPI00744889 | CDH1 | E-cadherin |
| IPI00290085 | CDH2 | Cadherin-2 |
| IPI00019580 | PLG | Plasminogen |
| IPI00022620 | SLURP1 | Secreted Ly-6/uPAR-related protein 1 |
| IPI00922213 | FN1 | cDNA FLJ53292, highly similar to Homo sapiens |
| | | fibronectin 1 (FN1), transcript variant 5, mRNA |
| IPI00031008 | TNC | Isoform 1 of Tenascin |
| IPI00872573 | C1RL | Uncharacterized protein |
| IPI00022895 | A1BG | Isoform 1 of Alpha-1B-glycoprotein |
| IPI00163207 | PGLYRP2 | Isoform 1 of N-acetylmuramoyl-L-alanine amidase |
| IPI00107731 | OSCAR | Isoform 6 of Osteoclast-associated immunoglobulin-like |
| | | receptor |
| IPI00166729 | AZGP1 | Zinc-alpha-2-glycoprotein |
| IPI00099670 | CEL | bile salt-activated lipase precursor |
| IPI00291867 | CFI | Complement factor I |
| IPI00216780 | CILP2 | Cartilage intermediate layer protein 2 precursor |
| IPI00395488 | VASN | Vasorin |
| IPI00645018 | PLAU | Isoform 2 of Urokinase-type plasminogen activator |
| IPI00553177 | SERPINA1 | Isoform 1 of Alpha-1-antitrypsin |
| IPI00029260 | CD14 | Monocyte differentiation antigen CD14 |
| IPI00024292 | LRP2 | Low-density lipoprotein receptor-related protein 2 |
| IPI00291262 | CLU | Isoform 1 of Clusterin |
| IPI00021885 | FGA | Isoform 1 of Fibrinogen alpha chain |
| IPI00026944 | NID1 | Isoform 1 of Nidogen-1 |
| IPI00006662 | APOD | Apolipoprotein D |
| IPI00291866 | SERPING1 | Plasma protease C1 inhibitor |
| IPI00176427 | CADM4 | Cell adhesion molecule 4 |
| IPI00017601 | CP | Ceruloplasmin |
| IPI00386879 | IGHA1 | cDNA FLJ14473 fis, clone MAMMA1001080, highly |
| | | similar to Homo sapiens SNC73 protein (SNC73) mRNA |
| IPI00021085 | PGLYRP1 | Peptidoglycan recognition protein 1 |
| IPI00103871 | ROBO4 | Isoform 1 of Roundabout homolog 4 |
| IPI00007221 | SERPINA5 | Plasma serine protease inhibitor |
| IPI00294713 | MASP2 | Isoform 1 of Mannan-binding lectin serine protease 2 |
| IPI00022488 | HPX | Hemopexin |
| IPI00645363 | IGHV4-31; | Putative uncharacterized protein DKFZp686P15220 |
| | IGHG1 | |
| IPI00153049 | MXRA8 | Isoform 2 of Matrix-remodeling-associated protein 8 |
| IPI00025476 | AMY1C; | Pancreatic alpha-amylase |
| | AMY1A; | |
| | AMY1B; | |
| | AMY2A | |
| IPI00291136 | COL6A1 | Collagen alpha-1(VI) chain |
| IPI00000073 | EGF | Isoform 1 of Pro-epidermal growth factor |
| IPI00009276 | PROCR | Endothelial protein C receptor precursor |
| IPI00004573 | PIGR | Polymeric immunoglobulin receptor |
| IPI00218192 | ITIH4 | Isoform 2 of Inter-alpha-trypsin inhibitor heavy chain H4 |
| IPI00160130 | CUBN | Cubilin |
| IPI00009950 | LMAN2 | Vesicular integral-membrane protein VIP36 |
| IPI00022463 | TF | Serotransferrin |
| IPI00215894 | KNG1 | Isoform LMW of Kininogen-1 |

1. Nucleic Acids

Embodiments concern polynucleotides or nucleic acid molecules relating to an OSA or high-risk OSA biomarker nucleic acid sequence in diagnostic applications. Certain embodiments specifically concern a nucleic acid that can be used to diagnose OSA or high-risk OSA based on the detection of an OSA biomarker. Nucleic acids or polynucleotides may be DNA or RNA, and they may be olignonucleotides (100 residues or fewer) in certain embodiments. Moreover, they may be recombinantly produced or synthetically produced.

Other embodiments concern the use of primers or hybridizable segments that may be used to identify and/or quantify OSA biomarkers, particularly in diagnostic methods. It is contemplated that the discussion below is relevant to embodiments concerning such methods and compositions related to diagnostic applications in the context of the OSA biomarkers.

These polynucleotides or nucleic acid molecules may be isolatable and purifiable from cells or they may be synthetically produced. In some embodiments, a nucleic acid targets or identifies an OSA biomarker. In other embodiments, a nucleic acid is an inhibitor, such as a ribozyme, siRNA, or shRNA.

As used in this application, the term “polynucleotide” refers to a nucleic acid molecule, RNA or DNA, that has been isolated free of total genomic nucleic acid. Therefore, a “polynucleotide encoding an OSA or high-risk OSA biomarker” refers to a nucleic acid sequence (RNA or DNA) that contains an OSA biomarker coding sequence, yet may be isolated away from, or purified and free of, total genomic DNA and proteins. An OSA biomarker inhibitor refers to an inhibitor of an OSA biomarker.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 2001; Ausubel, 1996). There may be times when the full or partial genomic sequence is used. Alternatively, cDNAs may be advantageous because it represents coding regions of a polypeptide and eliminates introns and other regulatory regions. In certain embodiments, nucleic acids are complementary or identical to all or part of cDNA encoding sequences.

The term “gene” is used for simplicity to refer to a functional protein, polypeptide, or peptide-encoding nucleic acid unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule hybridizing to all or part of a nucleic acid sequence may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more (or any range derivable therein) nucleotides, nucleosides, or base pairs of a sequence.

Accordingly, sequences that have or have at least or at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, and any range derivable therein, of nucleic acids that are identical or complementary to a nucleic acid sequence of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 contiguous bases (or any range derivable therein) of the identified biomarkers are contemplated as part of the invention.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

### C. SAMPLES

Urine is a highly desirable biological fluid for diagnostic testing because of its ease of collection and widespread use in clinical laboratories. Biomarker discovery strategies in urine, however, have been hindered by problems associated with reproducibility and inadequate standardization of proteomic protocols. Despite these concerns, urinary proteomics analyses have been leveraged to identify numerous candidate biomarkers of a broad range of human disorders, that have included, but are not limited to renal disease, diabetes, atherosclerosis, Alzheimer's disease, and cancer (Soggiu, 2012; Zimmerli, 2008; Riaz, 2010; Zengi, 2012; Huttenhain, 2012; Zoidakis, 2012; Zurbig, 2012; Siwy, 2011). In some embodiments, the sample may be a sample of urine, saliva, tears, or serum/plasma.

### D. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials And Methods

Patient Information—

Children (ages 2-12 years) clinically referred for evaluation of OSA underwent an overnight polysomnographic evaluation at the University of Chicago Pediatric Sleep Laboratory. Healthy children were recruited from schools or well-child clinics. Exclusion criteria for all subjects included the presence of significant genetic or craniofacial syndromes, diabetes, cystic fibrosis, cancer, or treatment with oral corticosteroids, antibiotics, or anti-inflammatory medications. All parents completed a detailed intake clinical questionnaire. Height, weight and vital signs were recorded for each child, and body mass index (BMI) z-score was calculated on the basis of CDC 2000 growth standards (www.cdc.gov/growthcharts) and using online software (www.cdc.gov/epiinfo). A BMI z-score exceeding 1.65 (0.95th percentile) was considered as fulfilling criteria for obesity. The study was approved by the institutional review boards at the University of Chicago (IRB 10-708A); informed consent and, when appropriate, assents for minors were obtained.

Overnight Polysomnography—

All subjects underwent an overnight polysomnography using standard methods (Montgomery-Downs, 2006). The PSG studies were scored as per the 2007 American Association of Sleep Medicine guidelines for the scoring of sleep and associated events (Iber, 2007). The obstructive apnea-hypopnea index (AHI) was defined as the number of obstructive apneas and hypopneas per hour of total sleep time.

Urine Collection—

Mid-stream urine specimens were collected in the evening just before bedtime and as the first void in the morning after awakening. Samples (20 ml) were collected into tubes containing phenylmethylsulfonyl fluoride (PMSF, 2 mM final concentration), and immediately stored at −80° C. until analysis.

Preparation of Soluble Urine Proteins for Mass Spectrometry (MS)—

Urine (10 mL) was thawed quickly at 37° C., vortexed for 90 s, and centrifuged (500×g, 4° C.) for 5 min. Supernatants were centrifuged at 12,000×g, 4° C. for 20 min to remove urinary sediment, and incubated with 1 mL ProteinG magnetic beads (Millipore) for 30 min at 20° C. Depletion of IgG was performed according to the manufacturer's protocol. IgG-depleted urine samples were precipitated using TCA/DOC as previously described (Thongboonkerd, 2006; Becker, 2010). Briefly, urine was supplemented with 0.02% sodium deoxycholate and 20% trichloroacetic acid, and incubated overnight with rocking at 4° C. Proteins were harvested by centrifugation (18,000×g for 60 min at 4° C.). The protein pellet was washed twice with ice-cold acetone, and reconstituted in 0.1% RapiGest (Waters Corp.), 250 mM ammonium bicarbonate, pH 8.8. Protein concentration was determined by the Bradford assay with albumin as a standard. Samples (90 μg) were incubated with α-human albumin-coupled magnetic beads (90 μL, Millipore) and depletion was performed according to the manufacturer's protocol. Samples were reduced, alkylated, and digested overnight at 37° C. with sequencing-grade trypsin (1:50, w/w, trypsin/protein; Promega). Tryptic digests were mixed with acetic acid (1:1, v/v) and subjected to solid-phase extraction on a C18 column (HLB, 1 mL; Waters Corp.) according to the manufacturer's protocol. Fractions containing peptides were dried under vacuum and resuspended in 0.3% formic acid, 5% acetonitrile (0.4 mg/mL) for LC-MS/MS analysis.

Liquid Chromatography-Electrospray Ionization-Tandem Mass Spectrometry (LC-ESI-MS/MS)—

Tryptic digests (1.5 μg) were loaded directly onto 2 cm C18 trap column (packed in-house), washed with 10 μl of solvent A (5% acetonitrile, 0.1% formic acid), and eluted on a 15 cm long, 75 μM reverse phase capillary column (ProteoPep™ II C18, 300 Å, 5 μm size, New Objective, Woburn Mass.). Peptides were separated at 300 nL/min over a 180 minute linear gradient from 5% to 35% buffer B (95% acetonitrile, 0.1% formic acid) on a Proxeon Easy n-LC II (Thermo Scientific, San Jose, Calif.). Mass spectra were acquired in the positive ion mode, using electrospray ionization and a linear ion trap mass spectrometer (LTQ Orbitrap Velos®, Thermo Scientific, San Jose, Calif.). The mass spectrometer was operated in data dependent mode, and for each MS1 precursor ion scan, the ten most intense ions were selected from fragmentation by CID (collision induced dissociation). Other parameters for mass spectrometry analysis included: resolution of MS1 was set at 60,000, normalized collision energy 35%, activation time 10 ms, isolation width 1.5, and the +1 and +4 and higher charge states were rejected.

Peptide and Protein Identification—

MS/MS spectra were searched against the International Protein Index human (v3.87, 91464 entries) primary sequence database (Kersey, 2004) using Sorcerer™-SEQUEST® (version v. 3.5,) (Sage-N Research, Milpitas, Calif.). Search parameters included semi-enzyme digest with trypsin (after Arg or Lys) with up to 2 missed cleavages. SEQUEST® was searched with a parent ion tolerance of 50 ppm and a fragment ion mass tolerance of 1 amu with fixed Cys alkylation, and variable Met oxidation. SEQUEST results were further validated with PeptideProphet (Keller, 2002) and ProteinProphet (Nesvizhskii, 2003), using an adjusted probability of ≥0.90 for peptides and ≥0.96 for proteins. Search results were further processed by the Computational Protemics Analysis System (CPAS) (Rauch, 2006) prior to statistical analysis (see below). Proteins considered for analysis had to be identified in at least 70% of individuals in at least one patient group (eg. healthy girls, or boys with OSA). When MS/MS spectra could not differentiate between protein isoforms, all were included in the analysis.

Protein Quantification and Statistical Analysis—

Proteins detected by LC-MS/MS were quantified by spectral counting (the total number of MS/MS spectra detected for a protein; (Liu, 2007)). Differences in relative protein abundance were assessed with the t-test and G-test (Becker, 2010; Becker, 2012; Old, 2005). Permutation analysis was used to empirically estimate the FDR (Benjamini, 1995). Significance cutoff values for the G-statistic and t-test were determined using PepC (Heinecke, 2010), a software package that maximizes the number of differentially expressed proteins identified for a given FDR.

ELISA—

Urine samples were thawed rapidly at 37° C. and clarified by centrifugation at 500×g for 10 min. Protein levels in resultant supernatants were quantified using commercially available ELISAs for DPP4 (Abnova; KA0141), AZGP1 (Abnova; KA1689), CP (Assaypro; EC4101-1), HPX (Innovative Research, Inc.; IRKTAH2562), and creatinine (Abcam; ab65340) according to the manufacturer's protocols. All protein levels were standardized to urine creatinine levels (Gardfe, 2004) and statistical significance between the groups was assessed by a two-tailed, Student's t-test.

Functional Annotation—

Functional enrichments in Gene Ontology annotations in the urine proteome or differentially expressed putative urine biomarkers (relative to the entire human genome) were identified using the Bingo 2.0 plugin in Cytoscape (V2.8.2) (Maere, 2005). Statistical significance was assessed using the hypergeometric test (p<0.05) with Benjamini-Hochberg correction (Benjamini, 1995) and functional categories with ≥5 proteins were considered.

### Example 2

### Proteomics Workflow for Urine Biomarker Discovery

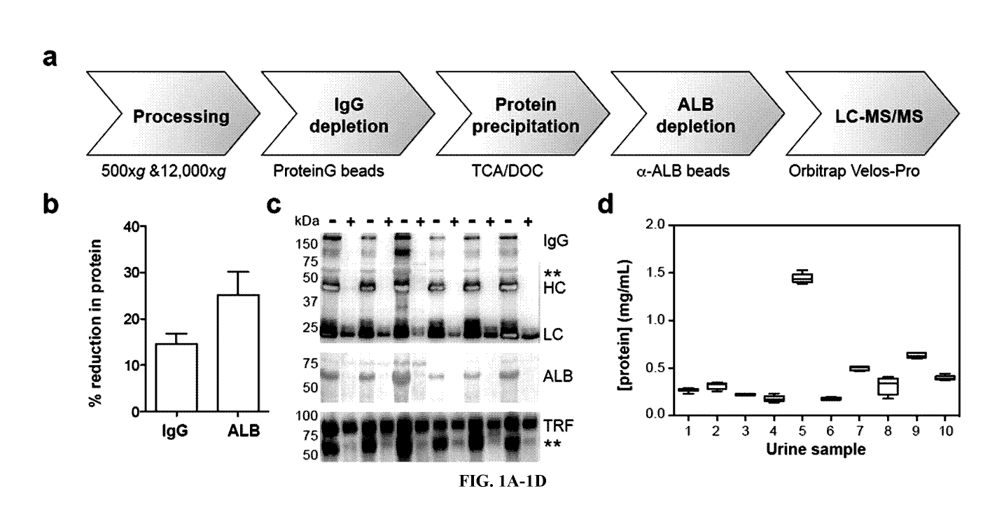

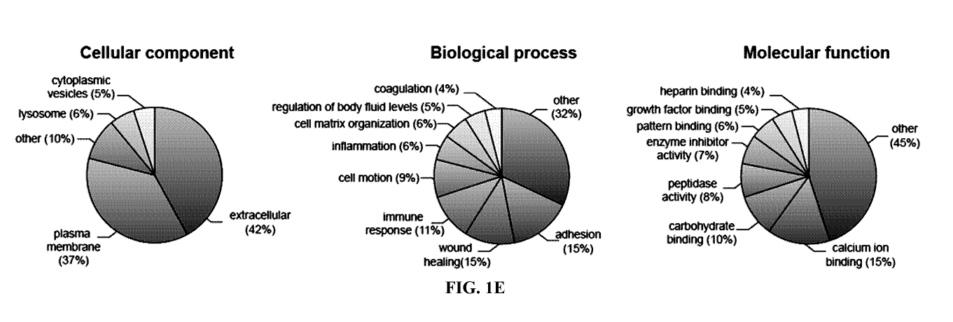

The inventors developed a 4-step procedure involving: i) centrifugation to remove particulate material and urinary sediment, ii) depletion of IgG and albumin (ALB) to facilitate deeper proteome coverage, iii) protein precipitation to concentrate urine proteins and remove interfering substances, and iv) mass spectrometric analysis by LC-MS/MS (FIG. 1a).

ALB and IgG are highly abundant urine proteins (40-60% of total urinary protein) that interfere with detection of low abundance species and complicate quantification in label-free proteomic approaches (Kushnir, 2009). Magnetic beads were carefully titrated to maximize depletion of ALB and IgG (FIG. 1b,c) and minimize non-specific loss of unrelated proteins, as assessed by loss of serotransferrin (TRF) levels (FIG. 1c). Since proteins are more efficiently precipitated in concentrated solutions (due to molecular crowding), the inventors depleted ALB after protein precipitation. However, IgG depletion was incompatible with the buffer (0.1% RapiGest) used to solubilize protein pellets, and was therefore performed prior to precipitation.

The inventors incorporated a method involving tricholoroaceteic acid and deoxycholate (TCA/DOC; (Thongboonkerd, 2006; Becker, 2010)) because it is well suited for precipitating proteins out of dilute solutions. The reproducibility of this method within and across samples was interrogated by precipitating 6 aliquots of the same urine sample collected from each of 10 subjects. This approach yielded highly reproducible results (6% CV, intra-sample) over a wide range of urinary protein concentrations (FIG. 1d).

To test the reproducibility of the proteomics workflow, urine samples from 28 children were processed and subjected to LC-MS/MS analysis. Based on a minimum of 2 unique peptide identifications per protein, the approach reliably identified 505±10 proteins per sample. Moreover, variation in sample depth, the number of high quality peptide identifications per run, was minimal (10,053±237 peptides) indicating that the method was robust and reproducible.

### Example 3

### Gender and Diurnal Effects Introduce Variability into the Urine Proteome of Healthy Children

The inventors collected morning and bedtime samples from healthy boys (N=7) and girls (N=6). Healthy children (ages 2-12 years) were selected by a priori excluding participants with genetic or craniofacial syndromes, diabetes, cystic fibrosis, or cancer. Additional exclusion criteria included chronic use of medications, steroids, or immunotherapy drugs.

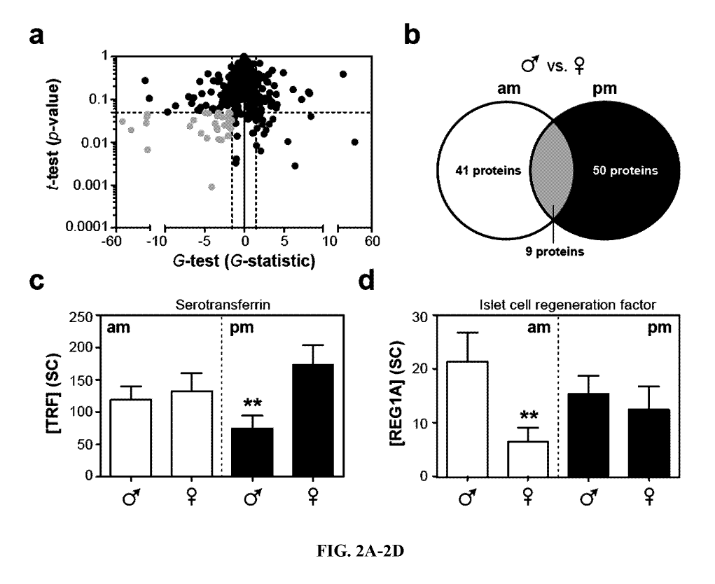

Samples were processed through the proteomics workflow (see FIG. 1), and subjected to LC-MS/MS analysis. Proteins were quantified by spectral counting (Liu, 2004), and statistically significant changes in protein levels were identified using a combination of the t-test and G-test (Becker, 2010; Becker, 2012; Old, 2005) using cutoffs that minimized the false discovery rate (Benjamini, 1995; Heinecke, 2010). A representative analysis is provided in FIG. 2a, which demonstrates the detection of gender-regulated proteins in morning urine samples upon application of the stringent statistical criteria (G-test: G≥1.5 or G≤−1.5; t-test: α=0.05; FDR<0.05).

Using this approach, the inventors detected substantial differences in the urinary proteome of healthy boys and girls, both in morning (˜7%; 50 of 750 proteins) and bedtime (8%; 41 of 750) samples (FIG. 2a,b, Tables 2A and 2B). Tables 2A and 2B indicates data illustrating the gender and diurnal effects on the urinary proteome of healthy children. A list of the statistically significant, gender-regulated proteins detected in morning and bedtime urine samples of healthy children are represented in Tables 3A and B. Results of the t-test and G-test are also presented.

TABLE 3A
Gender effects in bedtime (pm) samples

| IPI | Uniprot | Entrez | Gene | G-test | T-test |
|---|---|---|---|---|---|
| IPI00022463 | P02787\|Q06AH7\|A0PJA6\|B4DI57\|O43890\| | 7018 | TF | −40.31 | 0.0269 |
| | Q9UHV0\|Q53H26\|Q1HBA5\|B4E1B2\|B4DEX9\| | | | | |
| | Q9NQB8 | | | | |
| IPI00553177 | E9KL23\|Q0PVP5\|Q53XB8\|Q96BF9\|B2RDQ8\| | 5265 | SERPINA1 | −17.61 | 0.0035 |
| | Q13672\|Q5U0M1\|Q7M4R2\|P01009\|Q9P1P0\| | | | | |
| | Q9UCM3\|A6PX14\|Q9UCE6\|Q96ES1\|Q86U19\| | | | | |
| | Q86U18 | | | | |
| IPI00453473 | Q0VAS5\|B2R4R0\|P02305\|P02304\|A2VCL0\| | 8361\|8360\|8363\| | HIST1H4C\|HIST1H4B\|HIST1H4A\| | −10.47 | 0.0182 |
| | Q6DRA9\|Q6B823\|P62805\|Q6FGB8\|Q6NWP7 | 8362\|8365\|8364\| | HIST4H4\|HIST1H4F\|HIST1H4E\| | | |
| | | 8367\|8366\|8368\| | HIST1H4D\|HIST1H4K\|HIST1H4J\| | | |
| | | 8359\|8370\|8294\| | HIST1H4I\|HIST1H4H\|HIST1H4L\| | | |
| | | 554313\|121504 | HIST2H4B\|HIST2H4A | | |
| IPI00383164 | Q8WY24 | | | −7.65 | 0.0149 |
| IPI00305457 | Q9P173 | | | −7.46 | 0.0155 |
| IPI00003269 | Q562X8\|Q562S9\|B2RPJ1\|Q562R2\|Q562R1 | 345651 | ACTBL2 | −4.72 | 0.0042 |
| IPI00246058 | Q9P2H2\|Q8WUM4\|Q9BX86\|Q9NUN0\|Q9UKL5 | 10015 | PDCD6IP | −4.48 | 0.0390 |
| IPI00219018 | P04406\|Q0QET7\|Q2TSD0\|Q53X65\|P00354 | 2597 | GAPDH | −4.10 | 0.0049 |
| IPI00306322 | Q14052\|Q548C3\|Q66K23\|P08572\|Q5VZA9\| | 1284 | COL4A2 | −3.97 | 0.0495 |
| | B4DH43 | | | | |
| IPI00012540 | Q6SV49\|B3KQS1\|Q6SV53\|Q6SV52\|Q6SV51\| | 8842 | PROM1 | −3.78 | 0.0361 |
| | Q6SV50\|O43490\|Q96EN6 | | | | |
| IPI00301395 | O75225\|Q9NZ90\|Q6UX20\|Q9HB41\|Q9H3G5\| | 54504 | CPVL | −3.45 | 0.0083 |
| | Q8NBL7\|A4D1A4\|Q96AR7\|Q75MM4\|B3KW79 | | | | |
| IPI00034319 | Q9NYQ9\|O60888\|Q5JXM9\|Q3B784\|A2BEL4\| | 51596 | CUTA | −2.93 | 0.0066 |
| | A2AB26\|Q5SU05 | | | | |
| IPI00029751 | Q8N466\|A8K0H9\|Q14030\|Q7M4P0\|Q12860\| | 1272 | CNTN1 | −2.85 | 0.0042 |
| | Q12861\|A8K0Y3 | | | | |
| IPI00299086 | O00173\|O43391\|O00560\|B2R5Q7\|B4DUH3\| | 6386 | SDCBP | −2.62 | 0.0151 |
| | Q14CP2\|B7ZLN2 | | | | |
| IPI00028911 | Q14118\|Q969J9\|A8K6M7 | 1605 | DAG1 | −2.62 | 0.0095 |
| IPI00022290 | P60022\|Q09753\|Q86SQ8 | 1672 | DEFB1\|HBD1 | −2.51 | 0.0048 |
| IPI00178926 | P01591 | 3512 | IGJ | −2.49 | 0.0453 |
| IPI00020687 | P00995 | 6690 | SPINK1 | −2.49 | 0.0039 |
| IPI00002406 | P50895\|A8MYF9\|A9YWT5\|A9YWT6\|Q86VC7 | 4059 | BCAM | −2.45 | 0.0195 |
| IPI00295542 | Q02818\|B4DZX0\|Q9BUR1\|B2RD64\|Q7Z4J7\| | 4924 | NUCB1 | −2.43 | 0.0468 |
| | B3KUR6\|Q53GX6\|Q15838\|A8K7Q1\|Q96BA4 | | | | |
| IPI00064262 | Q96JQ0\|O15098 | 8642 | DCHS1 | −2.29 | 0.0032 |
| IPI00013446 | O43653\|Q6UW92\|D3DWI6 | 8000 | PSCA | −2.09 | 0.0393 |
| IPI00016786 | P25763\|P21181\|P60953\|Q9UDI2\|Q7L8R5 | 998 | CDC42 | −2.06 | 0.0453 |
| IPI00017567 | P17813\|A8K2X4\|B7Z6Y5\|Q14926\|Q5T9C0\| | 2022 | ENG | −1.87 | 0.0466 |
| | Q96CG0\|Q14248 | | | | |
| IPI00018279 | Q59GD4\|P25940\|Q9NZQ6 | 50509 | COL5A3 | −1.78 | 0.0139 |
| IPI00003648 | O75465\|Q2M3D3\|Q15223\|Q9HBW2\|Q9HBE6 | 5818 | PVRL1 | −1.70 | 0.0032 |
| IPI00329538 | Q9UCA3\|Q16651 | 5652 | PRSS8 | −1.66 | 0.0346 |
| IPI00025846 | Q63HM4\|Q02487 | 1824 | DKFZp686P18250\|DSC2 | 1.56 | 0.0392 |
| IPI00017557 | Q1ZYW2\|Q6PD64\|Q4G124\|Q6FHJ7\|Q6FHM0\| | 6424 | SFRP4 | 1.61 | 0.0061 |
| | O14877\|B4DYC1\|Q05BG7 | | | | |
| IPI00742682 | A0PJC9\|Q15655\|Q99968\|P12270\|Q15624\| | 7175 | tpr\|TPR\|Tpr | 1.64 | 0.0271 |
| | Q9UE33\|Q5SWY0\|Q504U6\|Q58F23 | | | | |
| IPI00022937 | | | | 1.65 | 0.0208 |
| IPI00552186 | Q5HYH5 | | DKFZp313O211 | 1.73 | 0.0069 |
| IPI00022371 | B2R8I2\|P04196\|B9EK35\|Q68DR3\|D3DNU7 | 3273 | DKFZp779H1622\|HRG | 2.08 | 0.0186 |
| IPI00300786 | B7ZMD7\|Q53F26\|P04745\|A8K8H6\|Q5T083\| | 278\|276\|277 | AMY1A\|AMY1C\|AMY1B | 2.17 | 0.0082 |
| | A6NJS5\|Q13763 | | | | |
| IPI00021000 | P10451\|Q8NBK2\|Q15681\|A6XMV6\|Q15682\| | 6696 | SPP1 | 2.26 | 0.0177 |
| | Q96IZ1\|Q4W597\|Q15683 | | | | |
| IPI00024331 | Q8WXR1\|B9DI89\|Q6IB95\|Q92956\|Q96J31\| | 8764 | TNFRSF14 | 2.43 | 0.0028 |
| | Q9UM65 | | | | |
| IPI00026926 | Q02747 | 2980 | GUCA2A | 2.61 | 0.0122 |
| IPI00290856 | Q8TC18\|Q9Y5Y7\|Q9UNF4\|B2R672 | 10894 | LYVE1\|XLKD1 | 2.62 | 0.0469 |
| IPI00019954 | Q6IBD2\|Q540N7\|Q15828 | 1474 | CST6 | 2.62 | 0.0251 |
| IPI00397820 | Q9NWB4\|Q8IUN3\|B7WPD9\|E2QRL0\|Q6ZQR9\| | 55083 | KIF26B | 2.64 | 0.0184 |
| | Q2KJY2\|Q6ZUZ0\|Q8IVR1 | | | | |
| IPI00028030 | O14592\|Q53FR6\|A8K3I0\|Q16389\|Q16388\| | 1311 | COMP | 2.67 | 0.0095 |
| | P49747\|Q8N4T2\|Q2NL86 | | | | |
| IPI00001662 | B7ZLQ1 | | OPCML | 2.75 | 0.0211 |
| IPI00006662 | D3DNW6\|B2R579\|P05090\|Q6IBG6 | 347 | APOD | 2.77 | 0.0419 |
| IPI00024046 | B7Z9B1 | 1012 | | 2.82 | 0.0060 |
| IPI00553215 | Q5NV65 | | IGLV2-18 | 3.12 | 0.0105 |
| IPI00946928 | B5MDQ5\|C7S7U0\|F5GZN4\|A1L4H1\|C7S7T9 | 284297 | SSC5D | 3.19 | 0.0295 |
| IPI00015881 | P09603\|Q5VVF4\|B4DTX0\|Q5VVF3\|Q14086\| | 1435 | CSF1 | 3.40 | 0.0165 |
| | A8K6J5\|Q9UQR8\|Q13130\|Q14806 | | | | |
| IPI00024035 | A8K5H5\|Q9BWS0\|P55285 | 1004 | CDH6 | 3.61 | 0.0123 |
| IPI00374563 | O00468\|Q15952\|B3KMM7\|Q96IC1\|Q60FE1\| | 375790 | AGRN | 3.75 | 0.0278 |
| | Q5SVA2\|Q8N4J5\|Q7KYS8\|Q9BTD4\|Q5XG79 | | | | |
| IPI00302944 | Q5VYK2\|Q71UR3\|Q5VYK1\|Q15955\|Q99716\| | 1303 | COL12A1 | 3.81 | 0.0367 |
| | Q99715\|O43853 | | | | |
| IPI00011302 | P13987\|Q6FHM9 | 966 | CD59 | 3.85 | 0.0371 |
| IPI00292130 | A8K981\|Q9UIX8\|Q07507\|Q8N4R2 | 1805 | DPT | 4.48 | 0.0181 |
| IPI00299724 | A6NLM2\|Q8TB12\|Q9Y4V0\|O00241\|B2R8V0\| | 10326 | SIRPB1 | 5.42 | 0.0109 |
| | Q9H1U5\|Q5TFQ9\|Q5TFR0 | | | | |
| IPI00031065 | Q14UV0\|Q14UU9\|P24855 | 1773 | DNASE1 | 5.53 | 0.0342 |
| IPI00293539 | A8MZC8\|Q9UQ94\|B7WP28\|Q9UQ93\|A8K5D6\| | 1009 | CDH11 | 6.43 | 0.0488 |
| | Q15065\|P55287\|Q15066 | | | | |
| IPI00006705 | P11684\|Q9UCM4\|B2R5F2\|Q6FHH3\|Q9UCM2 | 7356 | SCGB1A1 | 10.19 | 0.0009 |
| IPI00018136 | Q53FL7\|P19320\|Q6NUP8\|A8K6R7 | 7412 | VCAM1 | 12.52 | 0.0332 |
| IPI00021447 | B3KXB7\|D3DT76\|P19961\|Q9UBH3 | 280 | AMY2B | 14.46 | 0.0061 |
| IPI00166729 | O60386\|Q5XKQ4\|P25311\|D6W5T8\|Q8N4N0 | 563 | AZGP1 | 21.82 | 0.0265 |

TABLE 3B
Gender effect in morning (am) samples

| IPI | Uniprot | Entrez | Gene | G-test | T-test |
|---|---|---|---|---|---|
| IPI00027462 | Q6FGA1\|Q9UCJ1\|Q9NYM0\|B2R4M6\|P06702\|D3DV36 | 6280 | S100A9 | −50.36 | 0.0305 |
| IPI00007047 | A8K5L3\|Q9UCM6\|Q9UC84\|Q9UC92\|Q5SY70\|P05109\|Q9UCJ0\|D3DV37 | 6279 | S100A8 | −37.70 | 0.0193 |
| IPI00019038 | B2R4C5\|Q13170\|P00695\|P61626\|Q9UCF8 | 4069 | LYZ | −15.37 | 0.0278 |
| IPI00296180 | Q5PY49\|B2R7F2\|Q969W6\|Q16618\|B4DPZ2\|Q96SE8\|Q53XS3\|Q15844\|P00749\| | 5328 | ATF\|PLAU | −14.74 | 0.0386 |
| | Q5SWW9 | | | | |
| IPI00220143 | Q75ME7\|Q0VAX6\|O43451\|Q8TE24\|Q86UM5 | 8972 | MGAM | −13.45 | 0.0067 |
| IPI00384938 | Q7Z351 | | DKFZp686N02209 | −13.38 | 0.0447 |
| IPI00383164 | Q8WY24 | | | −6.83 | 0.0237 |
| IPI00027745 | B2R6X2\|Q96CL9\|Q549U0\|P08236 | 2990 | GUSB | −6.36 | 0.0328 |
| IPI00003807 | B7Z552\|Q561W5\|P11117\|Q9BTU7 | 53 | ACP2 | −5.41 | 0.0161 |
| IPI00027827 | Q6FHA2\|Q16867\|B2R9V7\|Q5U781\|P08294 | 6649 | SOD3 | −4.91 | 0.0485 |
| IPI00001593 | B2R7B7\|P42785\|B5BU34 | 5547 | PRCP | −4.67 | 0.0124 |
| IPI00025512 | B2R4N8\|Q9UC31\|Q96EI7\|Q9UC35\|Q9UC34\|Q9UC36\|Q6FI47\|P04792\|Q96C20 | 3315 | HSPB1 | −4.46 | 0.0473 |
| IPI00034319 | Q9NYQ9\|O60888\|Q5JXM9\|Q3B784\|A2BEL4\|A2AB26\|Q5SU05 | 51596 | CUTA | −4.09 | 0.0009 |
| IPI00021439 | Q75MN2\|Q53G76\|Q53G99\|Q96B34\|P99021\|Q11211\|P02570\|Q96HG5\|P70514\| | 60 | PS1TP5BP1\|ACTB | −3.65 | 0.0275 |
| | Q1KLZ0\|Q8WVW5\|Q64316\|P60709\|Q53GK6 | | | | |
| IPI00005794 | B5MDX4\|Q9Y646\|B2RD88\|Q8NBZ1\|Q9Y5X6\|Q9UNM8 | 10404 | PGCP | −3.44 | 0.0222 |
| IPI00018278 | Q71UI9\|A6NN01\|Q59GV8\|Q6PK98 | 94239 | H2AFV | −3.37 | 0.0195 |
| IPI00646304 | Q9BVK5\|Q6IBH5\|A8K534\|P23284 | 5479 | PPIB | −3.36 | 0.0406 |
| IPI00103871 | Q9NWJ8\|A8K154\|Q8TEG1\|Q8WZ75\|Q96JV6\|Q9H718\|Q14DU7 | 54538 | ROBO4 | −3.25 | 0.0489 |
| IPI00152871 | B3KWI4\|Q7RTN7\|Q495Q6\|Q8TF66 | 131578 | LRRC15 | −3.22 | 0.0117 |
| IPI00025869 | Q53HF3\|Q6LER7\|P06280\|Q53Y83 | 2717 | GLA\|alpha-GalA | −3.19 | 0.0480 |
| IPI00027166 | P16035\|Q9UDF7\|Q16121\|Q93006 | 7077 | TIMP2 | −2.83 | 0.0113 |
| IPI00301395 | O75225\|Q9NZ90\|Q6UX20\|Q9HB41\|Q9H3G5\|Q8NBL7\|A4D1A4\|Q96AR7\|Q75MM4\| | 54504 | CPVL | −2.74 | 0.0387 |
| | B3KW79 | | | | |
| IPI00178926 | P01591 | 3512 | IGJ | −2.47 | 0.0251 |
| IPI00783024 | Q9UL88 | | | −2.06 | 0.0140 |
| IPI00016786 | P25763\|P21181\|P60953\|Q9UDI2\|Q7L8R5 | 998 | CDC42 | −2.04 | 0.0377 |
| IPI00010737 | Q9UC32\|P07204\|Q8IV29 | 7056 | THBD | −1.94 | 0.0142 |
| IPI00021302 | Q9UGT4\|Q9H5Y6 | 56241 | SUSD2 | −1.93 | 0.0480 |
| IPI00176427 | B2R7L5\|Q9Y4A4\|Q8NFZ8 | 199731 | CADM4 | −1.90 | 0.0295 |
| IPI00414896 | Q9BZ46\|Q9BZ47\|B2RDA7\|E1P5C3\|Q8TCU2\|O00584\|Q5T8Q0 | 8635 | RNASET2 | −1.86 | 0.0223 |
| IPI00025714 | P57078\|Q96KH0 | | RIPK4 | −1.79 | 0.0486 |
| IPI00178415 | Q53SM0\|Q9HA24\|Q6UWV4\|Q4ZG47\|Q75T13\|Q4G0R8\|Q6AW92 | 80055 | PGAP1 | −1.66 | 0.0211 |
| IPI00029723 | D3DN90\|Q549Z0\|A8K523\|Q12841 | 11167 | FSTL1 | −1.54 | 0.0331 |
| IPI00005690 | A8K491\|O15232\|Q4ZG02 | 4148 | MATN3 | 1.52 | 0.0323 |
| IPI00018019 | Q86YE7\|Q5VYK8\|A8K5C3\|Q9NW75\|Q5VYK7 | 55105 | GPATCH2 | 1.58 | 0.0084 |
| IPI00296608 | Q6P3T5\|A8K2T4\|P10643\|B2R6W1\|Q92489 | 730 | C7 | 1.64 | 0.0213 |
| IPI00387025 | P01597 | | | 1.74 | 0.0374 |
| IPI00152418 | Q14UF3\|Q8TD14\|D3DT86\|B1AP16 | | DAF\|CD55 | 1.82 | 0.0428 |
| IPI00644680 | Q96JG9 | 84627 | ZNF469 | 2.02 | 0.0391 |
| IPI00045839 | Q96SK8\|Q9HC86\|Q9HC87\|Q96BR8\|Q7KZR4\|Q9H6K3\|Q96SL5\|Q96SN3\|Q32P28 | 64175 | LEPRE1 | 2.11 | 0.0063 |
| IPI00018909 | Q96NX0\|E9PBB5\|Q9UDA5\|Q07654 | 7033 | TFF3 | 2.32 | 0.0265 |
| IPI00553138 | P63027\|Q9BUC2\|P19065 | 6844 | VAMP2 | 2.36 | 0.0331 |
| IPI00021841 | Q9UCS8\|Q6LDN9\|Q9UCT8\|A8K866\|P02647\|Q6Q785\|Q6LEJ8 | 335 | APOA1 | 2.53 | 0.0156 |
| IPI00024046 | B7Z9B1 | 1012 | | 2.77 | 0.0193 |
| IPI00387097 | P01605 | | | 3.19 | 0.0266 |
| IPI00022620 | P55000\|Q6PUA6\|Q53YJ6\|Q92483 | 57152 | SLURP1 | 3.64 | 0.0348 |
| IPI00553215 | Q5NV65 | | IGLV2-18 | 5.53 | 0.0104 |
| IPI00019954 | Q6IBD2\|Q540N7\|Q15828 | 1474 | CST6 | 6.45 | 0.0028 |
| IPI00009027 | Q2TBE1\|P05451\|Q0VFX1\|A8K7G6\|P11379\|Q4ZG28 | 5967 | REG1A | 8.45 | 0.0403 |
| IPI00022426 | Q9UC58\|P02760\|Q9UDI8\|Q5TBD7\|P78492\|P00977\|P78491\|Q2TU33\|P02759 | 259 | ITIL\|AMBP | 34.38 | 0.0101 |

Interestingly, the inventors observed poor overlap (<10%) between differentially expressed proteins in morning and bedtime samples, suggesting that gender-related differences were also highly sensitive to diurnal effects (FIG. 2b). For example, TRF levels were elevated in girls at bedtime, while islet cell regeneration factor (REG1A) was specifically increased in morning urine samples collected from boys (FIG. 2c).

In general, urine protein composition was more substantially influenced by gender over diurnal effects. Consistent with this finding, gene ontology analysis of the gender-regulated urinary proteome in healthy children revealed significant enrichments in functional annotations that are not classically associated with gender (cell adhesion, p=6.0×10⁻⁷; pattern binding, p=7.0×10⁻³; complement and coagulation cascades p=4.2×10⁻³). In sharp contrast, this approach failed to identify significance in more intuitive modules such as female pregnancy (p=0.11) or embryo implantation (p=0.11).

### Example 4

### Urine Biomarker Discovery of Pediatric OSA is Highly Dependent Upon Gender and Diurnal Effects

Children (ages 2-12 years) with moderate to severe OSA, as assessed by the polysomnography-derived criterion of apnea hypopnea index (AHI>5 events/hour total sleep time), were recruited along with age- and sex-matched controls. Their demographic characteristics were such that no statistically significant differences in age, sex, ethnicity, or BMI distribution were present (Table 4).

TABLE 4
Demographic and polysomnographic characteristics of subjects.

| | Control | OSA | t-test |
|---|---|---|---|
| | (N = 13) | (N = 14) | (p-value) |
| Age (years) | 7.5 ± 0.8 | 5.9 ± 0.6 | 0.11 |
| Gender (boy, girl) | 7, 6 | 7, 7 | N/A |
| BMI, z-score | 0.6 ± 0.3 | 1.2 ± 0.5 | 0.27 |
| AHI (events/hr/total sleep time) | 0.4 ± 0.1 | 23.3 ± 5.3 | 0.0002 |

Abbreviations: BMI = body mass index; AHI = obstructive apnea-hypopnea index; OSA = obstructive sleep apnea.
Where applicable, results are presented as means ± SEM.

Using stringent criteria for quality and reproducibility of protein detection, the mass spectrometric analyses of urine samples identified 742 urine proteins across all patient samples.

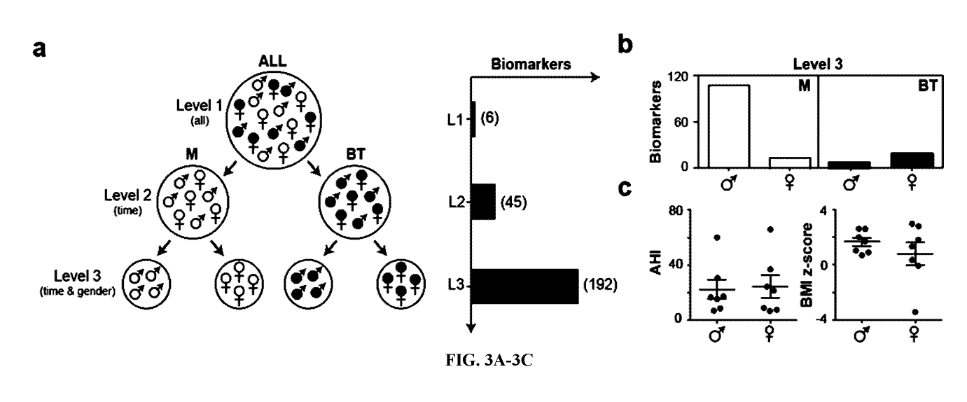

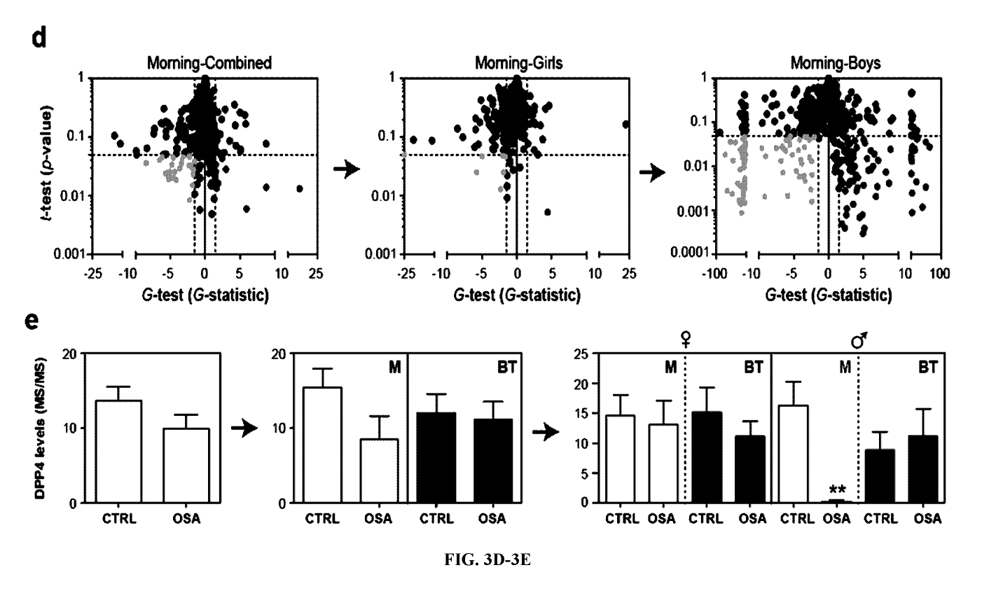

To investigate the impact of gender and diurnal variation on biomarker discovery, the inventors performed statistical analysis (using the t-test and G-test; (Becker, 2010; Old, 2005; Heinecke, 2010)) in three ways (FIG. 3a). In level 1 analysis, protein levels were averaged across morning and bedtime samples and groups were not differentiated according to gender. Level 2 analysis investigated morning and bedtime samples independently, while level 3 analysis treated samples in a collection time- and gender-dependent fashion (FIG. 3a).

Six candidate biomarkers of pediatric OSA were identified in level 1 analysis (Table 5A). Notably, orosomucoid 1 (ORM1), a protein that was initially identified in the previous OSA biomarker screen (Gozal, 2009), was also detected in this analysis. The statistical significance level for ORM1, however, barely cleared statistical thresholds, and subsequent ELISA measurements failed to validate this finding. A substantial increase in the number of biomarkers detected was evident when morning and bedtime samples were treated independently (level 2, 45 proteins) and a further, more dramatic, increase was visualized when gender was also accounted for in the analysis (level 3, 192 proteins) (FIG. 3a, Tables 5A-D). Tables 5A-D disclose the identification of urine biomarkers of pediatric OSA. Identification of differentially expressed urinary proteins in OSA relative to control samples. Results of levels 1 (all samples), 2 (corrected for diurnal effects), and 3 (corrected for both diurnal and gender effects) biomarker analysis along with corresponding t-test and G-test values are displayed.

TABLE 5A
Level 1 analysis (morning/bedtime measurements averaged, genders pooled)

| | | | Gene | | G- | |
|---|---|---|---|---|---|---|
| IPI | UniProt | Entrez | name | Description | test | T-test |
| IPI00160130 | Q7LC53\|B0YIZ4\|O60494\|Q5VTA6\|Q59ED | 8029 | CUBN | cDNA FLJ90747 fis, clone PLACE1011708, | −14.68 | 0.0143 |
| | 1\|B3KQM71\|Q96RU9 | | | highly similar to Cubilin\|Cubilin | | |
| | | | | variant\|Cubilin\|Intrinsic factor-vitamin B12 | | |
| | | | | receptor | | |
| IPI00291136 | Q9BSA8\|Q14040\|Q14041\|O00117\|Q16258\| | 1291 | COL6A1 | Collagen alpha-1(VI) chain\|Putative | −4.79 | 0.0452 |
| | O00118\|Q7Z645\|P12109\|Q8TBN2 | | | uncharacterized protein | | |
| IPI00022255 | B4DV64\|Q5VWG0\|O95362\|Q6UX06\|Q86T | 10562 | OLFM4 | Olfactomedin-4\|cDNA FLJ61420, highly | −2.01 | 0.0231 |
| | 22 | | | similar to Homo sapiens olfactomedin 4 | | |
| | | | | (OLFM4), mRNA | | |
| IPI00022429 | B7ZKQ5\|P02763\|Q8TC16\|Q5T539\|Q5U067 | 5004 | ORM1 | Alpha-1-acid glycoprotein 1 | 2.02 | 0.0216 |
| IPI00219684 | Q5VV93\|B2RAB6\|Q99957\|P05413\|Q6IBD7 | 2170 | FABP3 | FABP3 protein\|Fatty acid-binding protein, | 2.40 | 0.0215 |
| | | | | heart | | |
| IPI00555812 | Q53F31\|P02774\|B4DPP2\|Q16309\|Q16310\|Q | 2638 | GC | Vitamin D-binding protein | 3.93 | 0.0079 |
| | 6GTG1 | | | | | |

TABLE 5B
Level 2 analysis (morning/bedtime samples treated independently, genders pooled)

| | | | | | | |
|---|---|---|---|---|---|---|
| IPI | UniProt | Entrez | Gene name | Description | G-test | T-test |

Morning (am) samples

| | | | | | | |
|---|---|---|---|---|---|---|
| IPI00160130 | Q7LC53\|B0YIZ4\|O60494\|Q5VTA6\|Q59E | 8029 | CUBN | cDNA FLJ90747 fis, clone | −32.91 | 0.0130 |
| | D1\|B3KQM7\|Q96RU9 | | | PLACE1011708, highly similar to | | |
| | | | | Cubilin\|Cubilin variant\|Cubilin\|Intrinsic | | |
| | | | | factor-vitamin B12 receptor | | |
| IPI00009276 | Q14218\|Q9ULX1\|Q96CB3\|B2RC04\|Q9U | 10544 | PROCR | Endothelial protein C receptor | −8.48 | 0.0360 |
| | NN8\|Q6IB56 | | | | | |
| IPI00021885 | Q9BX62\|A8K3E4\|Q4QQH7\|D3DP14\|P02 | 2243 | FGA | cDNA FLJ78367, highly similar to Homo | −6.58 | 0.0405 |
| | 671\|D3DP15\|Q9UCH2 | | | sapiens fibrinogen, A alpha polypeptide | | |
| | | | | (FGA), transcriptvariant alpha, | | |
| | | | | mRNA\|Fibrinogen alpha chain | | |
| IPI00008787 | Q147691\|P54802 | 4669 | NAGLU\|ufHSD2 | Alpha-N-acetylglucosaminidase | −6.17 | 0.0457 |
| IPI00299738 | O14550\|A4D2D2\|B2R9E1\|Q15113 | 5118 | PCOLCE | Procollagen C-endopeptidase | −5.68 | 0.0242 |
| | | | | enhancer\|Procollagen C-endopeptidase | | |
| | | | | enhancer 1 | | |
| IPI00003919 | Q16770\|Q3KRG6\|Q16769\|Q53TR4 | 25797 | tmp_locus_46\| | Glutaminyl-peptide | −5.24 | 0.0208 |
| | | | QPCT | cyclotransfemse\|Glutaminyl-peptide | | |
| | | | | cyclotransferase (Glutaminyl cyclase), | | |
| | | | | isoform CRA_a | | |
| IPI00029275 | P08582\|Q9BQE2 | 4241 | MFI2 | Melanotransferrin | −5.22 | 0.0190 |
| IPI00027843 | P22891\|A6NMB4\|Q5JVF6\|Q15213\|Q5JV | 8858 | PROZ | Vitamin K-dependent protein Z | −5.08 | 0.0257 |
| | F5 | | | | | |
| IPI00029751 | Q8N466\|A8K0H9\|Q14030\|Q7M4P0\|Q128 | 1272 | CNTN1 | Contactin-1 | −4.78 | 0.0338 |
| | 60\|Q12861\|A8K0Y3 | | | | | |
| IPI00027827 | Q6FHA2\|Q16867\|B2R9V7\|Q5U781\|P082 | 6649 | SOD3 | Superoxide dismutase [Cu— | −4.59 | 0.0450 |
| | 94 | | | Zn]\|Extracellular superoxide dismutase | | |
| | | | | [Cu—Zn] | | |
| IPI00176427 | B2R7L5\|Q9Y4A4\|Q8NFZ8 | 199731 | CADM4 | Cell adhesion molecule 4 | −4.54 | 0.0202 |
| IPI00043992 | Q96K15\|Q96NY8 | 81607 | PVRL4 | Poliovirus receptor-related protein 4 | −4.35 | 0.0257 |
| IPI00022432 | Q9UBZ6\|Q6IB96\|P02766\|E9KL36\|Q549 | 7276 | TTR | Epididymis tissue sperm binding protein Li | −4.14 | 0.0187 |
| | C7\|Q9UCM9 | | | 4a\|Transthyretin | | |
| IPI00031065 | Q14UV0\|Q14UU9\|P24855 | 1773 | DNASE1 | Deoxyribonuclease\|Deoxyribonuclease-1 | −3.62 | 0.0291 |
| IPI00007800 | Q8N2J9\|B2R780\|Q5JT58\|Q9UKU9 | 23452 | ANGPTL2 | Angiopoietin-related protein 2\|cDNA | −3.43 | 0.0362 |
| | | | | FLJ90545 fis, clone OVARC1000410, | | |
| | | | | highly similar to Angiopoietin-related | | |
| | | | | protein 2\|cDNA, FLJ93320, highly similar | | |
| | | | | to Homo sapiens angiopoietin-like 2 | | |
| | | | | (ANGPTL2), mRNA | | |
| IPI00010949 | Q9HAT2\|B3KPB0\|Q9HAU7\|Q8IUT9\|Q9 | 54414 | SIAE | Sialate O-acetylesterase | −3.18 | 0.0454 |
| | NT71 | | | | | |
| IPI00328746 | B7ZLI0\|Q6X813\|Q17RL9\|Q86UN3 | 349667 | RTN4RL2 | Reticulon 4 receptor-like 2\|Reticulon-4 | −2.30 | 0.0327 |
| | | | | receptor-like 2 | | |
| IPI00019157 | D3DW77\|Q92675\|Q6UVK1 | 1464 | CSPG4 | Chondroitin sulfate proteoglycan 4 | −2.28 | 0.0468 |
| IPI00240345 | Q695G9\|Q86T13\|Q6PWT6\|Q8N5V5 | 161198 | CLEC14A | C-type lectin domain family 14 member A | −2.23 | 0.0086 |
| IPI00022255 | B4DV64\|Q5VWG0\|O95362\|Q6UX06\|Q8 | 10562 | OLFM4 | Olfactomedin-4\|cDNA FLJ61420, highly | −2.23 | 0.0467 |
| | 6T22 | | | similar to Homo sapiens olfactomedin 4 | | |
| | | | | (OLFM4), mRNA | | |
| IPI00102300 | Q9UIF2\|Q9HCN7\|Q9HCN6 | 51206 | GP6 | Platelet glycoprotein VI | −2.03 | 0.0326 |
| IPI00000024 | B4E2D8\|Q8IUP2\|Q08174 | 5097 | PCDH1 | cDNA F1159655, highly similar to | −1.80 | 0.0150 |
| | | | | Protocadherin-1\|Protocadherin-1 | | |
| IPI00179185 | O00520\|Q96MX2\|Q66K79 | 8532 | CPZ | Carboxypeptidase Z | −1.75 | 0.0231 |
| IPI00022039 | B7Z3R8\|O95660\|Q9UIB8\|B2R8T1\|Q5H9 | 8832 | CD84 | SLAM family member 5 | −1.66 | 0.0281 |
| | R1\|O15430\|Q9UIT7\|Q6FHA8\|O95266\|Q8 | | | | | |
| | WLP1\|Q8WWI8\|Q9UF04\|Q9UIB6\|Q9UI | | | | | |
| | B7 | | | | | |
| IPI00289275 | O75339\|B2R8F7\|Q8IYI5\|Q6UW99 | 8483 | CILP | Cartilage intermediate layer protein 1 | −1.51 | 0.0389 |
| IPI00298388 | Q49A94\|Q8NCJ9\|Q96FE7\|Q86YW2\|O00 | 113791 | PIK3IP1 | Phosphoinositide-3-kinase-interacting | 1.53 | 0.0340 |
| | 318 | | | protein 1 | | |
| IPI00289501 | O15240\|Q9UDW8 | 7425 | VGF | Neurosecretory protein VGF | 1.57 | 0.0257 |
| IPI00175092 | Q53SV6\|Q8WUU3\|Q8NC42\|Q8NBY5\|Q5 | 284996 | RNF149\|LOC | Putative uncharacterized protein | 1.59 | 0.0138 |
| | 3514\|Q8N5I8 | | 284996 | LOC284996\|E3 ubiquitin-protein ligase | | |
| | | | | RNF149 | | |
| IPI00022429 | B7ZKQ5\|P02763\|Q8TC16\|Q5T539\|Q5U0 | 5004 | ORM1 | Alpha-1-acid glycoprotein 1 | 1.68 | 0.0136 |
| | 67 | | | | | |
| IPI00922213 | Q14327\|Q7L553\|B4DTK1\|Q6PJE5\|Q9H3 | | FN1 | Putative uncharacterized protein | 1.87 | 0.0165 |
| | 82\|Q53S27\|B4DTH2 | | | FN1\|cDNA FLJ61165, highly similar to | | |
| | | | | Fibronectin\|FN1 protein\|Fibronectin | | |
| | | | | 1\|cDNA FLJ53292, highly similar to Homo | | |
| | | | | sapiens fibronectin 1 (FN1), transcript | | |
| | | | | variant 5, mRNA | | |
| IPI00013955 | Q9UE76\|Q9UE75\|Q9UQL1\|Q7Z552\|Q14 | 4582 | MUC1 | Mucin-1 | 2.45 | 0.0436 |
| | 876\|Q9Y4J2\|Q14128\|Q16437\|P13931\|P17 | | | | | |
| | 626\|P15941\|Q16615\|P15942\|Q16442\|Q9B | | | | | |
| | XA4 | | | | | |
| IPI00010343 | Q9UPR5\|B4DYQ9\|B4DEZ4 | 6543 | SLC8A2 | cDNA F1158526, highly similar to | 2.99 | 0.0284 |
| | | | | Sodium/calcium exchanger | | |
| | | | | 2\|Sodium/calcium exchanger 2 | | |
| IPI00219684 | Q5VV93\|B2RAB6\|Q99957\|P05413\|Q6IB | 2170 | FABP3 | FABP3 proteinFatty acid-binding protein, | 5.95 | 0.0060 |
| | D7 | | | heart | | |
| IPI00007778 | F6X5H7\|B2RBF5\|Q5VX51\|Q5VX50\|Q8T | 1486 | CTBS | cDNA PSEC0114 fis, clone | 8.79 | 0.0140 |
| | C97\|B3KQS3\|B4DQ98\|Q01459 | | | NT2RP2006543, highly similar to DI-N- | | |
| | | | | ACETYLCHITOBIASE (EC 3.2.1.-) | | |
| | | | | \|ICTBS protein\|Di-N- | | |
| | | | | acetylchitobiase\|cDNA FLJ55135, highly | | |
| | | | | similar to Di-N-acetylchitobiase (EC | | |
| | | | | 3.2.i.-)\|cDNA, FLJ95483, highly similar to | | |
| | | | | Homo sapiens chitobiase, di-N-acetyl- | | |
| | | | | (CTBS), mRNA\|Chitobiase, di-N-acetyl- | | |
| IPI00022620 | P55000\|Q6PUA6\|Q53YJ6\|Q92483 | 57152 | SLURP1 | Secreted Ly-6/uPAR-related protein 1 | 15.99 | 0.0133 |

Bedtime (pm) samples

| | | | | | | |
|---|---|---|---|---|---|---|
| IPI00555812 | Q53F31\|P02774\|B4DPP2\|Q16309\|Q16310 | 2638 | GC | Vitamin D-binding protein | 9.84 | 0.0034 |
| | \|Q6GTG1 | | | | | |
| IPI00170635 | B2R7H0\|Q8WVN6\|Q53G27\|O00466\|A8K | 6398 | SECTM1 | Secreted and transmembrane protein | 9.44 | 0.0409 |
| | 3U3\|Q53G63 | | | 1\|Secreted and transmembrane 1 precusor | | |
| | | | | variant\|cDNA FLJ77863, highly similar to | | |
| | | | | Homo sapiens secreted and transmembrane | | |
| | | | | 1 (SECTM1), mRNA | | |
| IPI00022488 | P02790\|B2R957 | 3263 | HPX | Hemopexin | 5.80 | 0.0209 |
| IPI00022432 | Q9UBZ6\|Q6IB96\|P02766\|E9KL36\|Q549 | 7276 | TTR | Epididymis tissue sperm binding protein Li | 5.63 | 0.0157 |
| | C7\|Q9UCM9 | | | 4a\|Transthyretin | | |
| IPI00008787 | Q14769\|P54802 | 4669 | NAGLU\|ufHSD2 | Alpha-N-acetylglucosaminidase | 4.89 | 0.0206 |
| IPI00022420 | D3DR38\|P02753\|Q9P178\|Q8WWA3\|Q5V | 5950 | RBP4 | Retinol-binding protein 4 | 4.32 | 0.0370 |
| | Y24\|O43479\|O43478 | | | | | |
| IPI00032258 | B0QZR6\|Q13160\|A7E2V2\|Q14033\|P0C0 | 720\|721 | C4A variant | Complement C4-A\|C4A variant | 3.97 | 0.0344 |
| | L4\|B7ZVZ6\|Q6P4R1\|B2RUT6\|Q5JQM8\| | | protein\|C4A | protein1Complement component 4A | | |
| | Q4LE82\|P01028\|Q9NPK5\|P78445\|Q1390 | | | (Rodgers blood group) | | |
| | 6\|Q14835\|Q9UIP5 | | | | | |
| IPI00021085 | O755941Q4VB36 | 8993 | PGLYRP1 | Peptidoglycan recognition protein 1 | 3.01 | 0.0373 |
| IPI00010949 | Q9HAT2\|B3KPB0\|Q9HAU7\|Q8IUT9\|Q9 | 54414 | SIAE | Sialate O-acetylesterase | 2.99 | 0.0064 |
| | NT71 | | | | | |
| IPI00744184 | Q96CJ0\|P15289\|B7XD04\|Q63HL5\|Q6ICI | 410 | ARSA\|DKFZp686 | Putative uncharacterized protein | 2.16 | 0.0193 |
| | 5\|1B2RCA6 | | G12235 | DKFZp686G12235\|Arylsulfatase A | | |

TABLE 5C
Level 3 analysis (morning/bedtime samples and genders treated independently—boys)

| | | | | | | |
|---|---|---|---|---|---|---|
| IPI | UniProt | Entrez | Gene name | Description | G-test | T-test |

Morning (am) samples

| | | | | | | |
|---|---|---|---|---|---|---|
| IPI00032328 | P01043\|P01042\|B4E1C2\|Q7M4P1\|B2RC | 3827 | KNG1 | Kininogen-1\|Kininogen 1, isoform CRA_b | −72.60 | 0.0187 |
| | R2\|A8K474\|Q6PAU9\|Q53EQ0 | | | | | |
| IPI00004573 | P01833\|Q8IZY7\|Q68D81 | 5284 | PIGR | Polymeric immunoglobulin receptor | −67.34 | 0.0028 |
| IPI00029260 | Q96FR6\|F1C4A7\|Q9UNS3\|Q96L99\|B2R | 929 | CD14 | Monocyte differentiation antigen CD14 | −57.39 | 0.0363 |
| | 888\|P08571\|Q53XT5 | | | | | |
| IPI00291136 | Q9BSA8\|Q14040\|Q14041\|O00117\|Q162 | 1291 | COL6A1 | Collagen alpha-1(VI) chain\|Putative | −50.80 | 0.0024 |
| | 58\|O00118\|O7Z645\|P12109\|Q8TBN2 | | | uncharacterized protein | | |
| IPI00218192 | Q15135\|Q14624\|Q9UQ54\|Q9P190 | 3700 | ITIH4 | Inter-alpha-trypsin inhibitor heavy chain | −48.66 | 0.0136 |
| | | | | H4 | | |
| IPI00009950 | Q53HH1\|Q12907\|A8K7T4 | 10960 | LMAN2 | cDNA FLJ75774, highly similar to Homo | −41.87 | 0.0351 |
| | | | | sapiens lectin, marmose-binding 2 | | |
| | | | | (LMAN2), mRNA\|Vesicular integral- | | |
| | | | | membmne protein VIP36 | | |
| IPI00294713 | Q9H498\|Q9UMV3\|Q9ULC7\|Q96QG4\|O | 10747 | MASP2 | Mannan-binding lectin serine protease 2 | −34.82 | 0.0042 |
| | 75754\|Q9UC48\|O00187\|Q9H499\|Q5TEQ | | | | | |
| | 5\|Q9BZH0\|Q5TER0\|A8K458\|A8MWJ2\| | | | | | |
| | Q9UBP3\|Q9Y270 | | | | | |
| IPI00000073 | E9PBF0\|P01133\|B4DRK7\|Q52LZ6 | 1950 | EGF | Pro-epidermal growth factor | −30.27 | 0.0017 |
| IPI00022488 | P02790\|B2R957 | 3263 | HPX | Hemopexin | −27.44 | 0.0086 |
| IPI00291866 | A6NMU0\|Q9UC49\|Q96FE0\|P05155\|A8 | 710 | SERPING1 | Plasma protease C1 inhibitor\|Epididymis | −26.09 | 0.0036 |
| | KAI9\|E9KL26\|Q7Z455\|Q16304\|B2R6L5\| | | | tissue protein Li 173 | | |
| | Q59EI5\|Q547W3\|Q9UCF9 | | | | | |
| IPI00009028 | P05452\|B2R582\|Q6FGX6 | 7123 | CLEC3B | Tetranectin\|cDNA, FLJ92374, highly | −26.01 | 0.0014 |
| | | | | similar to Homo sapiens C-type lectin | | |
| | | | | domain family 3, member B (CLEC3B), | | |
| | | | | mRNA | | |
| IPI00006662 | D3DNW6\|B2R579\|P05090\|Q6IBG6 | 347 | APOD | Apolipoprotein D | −25.64 | 0.0239 |
| IPI00299738 | O14550\|A4D2D2\|B2R9E1\|Q15113 | 5118 | PCOLCE | Procollagen C-endopeptidase | −23.92 | 0.0214 |
| | | | | enhancer\|Procollagen C-endopeptidase | | |
| | | | | enhancer 1 | | |
| IPI00027843 | P22891\|A6NMB4\|Q5JVF6\|Q15213\|Q5JV | 8858 | PROZ | Vitamin K-dependent protein Z | −23.04 | 0.0009 |
| | F5 | | | | | |
| IPI00021085 | O75594\|Q4VB36 | 8993 | PGLYRP1 | Peptidoglycan recognition protein 1 | −21.41 | 0.0262 |
| IPI00395488 | Q6UXL4\|Q6UXL5\|Q96CX1\|Q6EMK4 | 114990 | VASN | Vasorin | −21.17 | 0.0017 |
| IPI00018953 | Q53TN1\|P27487 | 1803 | DPP4 | Dipeptidyl peptidase 4 | −20.33 | 0.0153 |
| IPI00293539 | A8MZC8\|Q9UQ94\|B7WP28\|Q9UQ931A | 1009 | CDH11 | Cadherin-11 | −19.41 | 0.0246 |
| | 8K5D6\|Q15065\|P55287\|Q15066 | | | | | |
| IPI00027235 | Q9UC75\|Q9NTQ3\|O95414\|O75882\|Q9U | 8455 | ATRN | Uncharacterized protein\|Attractin | −19.25 | 0.0188 |
| | DF5\|Q9NU01\|A8KAE5\|Q9NZ58\|O60295 | | | | | |
| | \|Q3MIT3\|Q9NZ57\|Q5VYW3\|C9IZD4\|Q5 | | | | | |
| | TDA4\|Q5TDA2\|Q9NTQ4 | | | | | |
| IPI00026314 | A8MUD1\|B7Z9A0\|P06396\|Q8WVV7\|B7 | 2934 | GSN | Gelsolin (Amyloidosis, Finnish | −18.95 | 0.0436 |
| | Z373\|Q5T0I2\|B7Z6N2 | | | type)\|cDNA FLJ56154, highly similar to | | |
| | | | | GelsolinlcDNA FLJ56212, highly similar | | |
| | | | | to Gelsolin\|Gelsolin | | |
| IPI00216780 | Q6NV88\|Q8IUL8\|Q8WV21\|Q8N4A6\|B2 | 148113 | CILP2 | cDNA, FLJ94946, highly similar to Homo | −18.69 | 0.0026 |
| | RAJ0 | | | sapiens cartilage intermediate layer protein | | |
| | | | | 2 (CILP2), mRNA\|Cartilage intermediate | | |
| | | | | layer protein 2 | | |
| IPI00021885 | Q9BX62\|A8K3E4\|Q4QQH7\|D3DP14\|P0 | 2243 | FGA | cDNA FLJ78367, highly similar to Homo | −18.53 | 0.0163 |
| | 2671\|D3DP15\|Q9UCH2 | | | sapiens fibrinogen, A alpha polypeptide | | |
| | | | | (FGA), transcriptvariant alpha, | | |
| | | | | mRNA\|Fibrinogen alpha chain | | |
| IPI00060800 | Q96DA0\|C3PTT6\|B2R4F6\|A6NIY1\|Q6U | 124220 | PAUF\| | Zymogen granule protein 16 homolog | −17.51 | 0.0227 |
| | W28 | | ZG16B | B\|Pancreatic adenocarcinoma upregulated | | |
| | | | | factor | | |
| IPI00176427 | B2R7L5\|Q9Y4A4\|Q8NFZ8 | 199731 | CADM4 | Cell adhesion molecule 4 | −17.33 | 0.0021 |
| IPI00022661 | Q92692\|Q96J29\|Q6IBI6\|O75455\|Q7Z456 | 5819 | PVRL2 | Poliovirus receptor-related protein | −16.66 | 0.0454 |
| | | | | 2\|Poliovirus receptor related 2 | | |
| IPI00291262 | Q5HYC1\|Q2TU75\|B3KSE6\|Q7Z5B9\|B2 | 1191 | CLU | Clusterin | −16.20 | 0.0096 |
| | R9Q1\|P11381\|P11380\|P10909 | | | | | |
| IPI00221224 | Q6GT90\|Q8IVL7\|B4DP01\|Q59E93\|Q167 | 290 | ANPEP\| | cDNA FLJ56158, highly similar to | −16.15 | 0.0111 |
| | 28\|Q8IUK3\|Q8IVH3\|P15144\|Q71E46\|B4 | | CD13 | Aminopeptidase N (EC | | |
| | DV63\|B4DPH5\|B4DP96\|Q9UCE0 | | | 3.4.11.2)Membrane alanine | | |
| | | | | aminopeptidase variant\|Uncharacterized | | |
| | | | | protein\|Aminopeptidase N\|cDNA | | |
| | | | | FLJ56120, highly similar to | | |
| | | | | Aminopeptidase N (EC 3.4.11.2)\|cDNA | | |
| | | | | FLJ55496, highly similar to | | |
| | | | | Aminopeptidase N (EC 3.4.11.2) | | |
| IPI00291867 | Q6LAM0\|P05156\|O60442 | 3426 | CFI | Complement factor I\|Light chain of factor I | −15.00 | 0.0147 |
| IPI00003919 | Q16770\|Q3KRG6\|Q16769\|Q53TR4 | 25797 | tmp_locus_ | Glutaminyl-peptide | −14.35 | 0.0121 |
| | | | 46\|QPCT | cyclotransferase\|Glutaminyl-peptide | | |
| | | | | cyclotransferase (Glutaminyl cyclase), | | |
| | | | | isoform CRA_a | | |
| IPI00099670 | P19835\|Q9UP41\|Q16398\|O75612\|B4DS | 1056 | CEL | cDNA FLJ51297, highly similar to Bile | −13.80 | 0.0464 |
| | X9\|Q9UCH1\|Q5T7U7 | | | salt-activated lipase (EC 3.1.1.3)\|Bile salt- | | |
| | | | | dependent lipase oncofetal isoform\|Bile | | |
| | | | | salt-activated lipase | | |
| IPI00031065 | Q14UV0\|Q14UU9\|P24855 | 1773 | DNASE1 | Deoxyribonuclease\|Deoxyribonuclease-1 | −13.80 | 0.0044 |
| IPI00015525 | Q504V7\|B4E3H8\|Q6P2N2\|Q9H8L6 | 79812 | MMRN2 | Multimerin-2\|cDNA FLJ54082, highly | −13.66 | 0.0046 |
| | | | | similar to Multimerin-2 | | |
| IPI00043992 | Q96K15\|Q96NY8 | 81607 | PVRL4 | Poliovirus receptor-related protein 4 | −13.66 | 0.0332 |
| IPI00022432 | Q9UBZ6\|Q6IB96\|P02766\|E9KL36\|Q549 | 7276 | TTR | Epididymis tissue sperm binding protein Li | −13.27 | 0.0042 |
| | C7\|Q9UCM9 | | | 4a\|Transthyretin | | |
| IPI00022290 | P60022\|Q09753\|Q86SQ8 | 1672 | DEFB1\| | Beta-defensin-1\|Beta-defensin 1 | −13.27 | 0.0053 |
| | | | HBD1 | | | |
| IPI00102300 | Q9UIF2\|Q9HCN7\|Q9HCN6 | 51206 | GP6 | Platelet glycoprotein VI | −13.13 | 0.0032 |
| IPI00240345 | Q695G9\|Q86T13\|Q6PWT6\|Q8N5V5 | 161198 | CLEC14A | C-type lectin domain family 14 member A | −12.91 | 0.0015 |
| IPI00153049 | Q5TA39\|Q96KC3\|Q9BRK3 | 54587 | MXRA8 | Matrix-remodeling-associated protein 8 | −12.88 | 0.0286 |
| IPI00029658 | A8KAJ3\|Q541U7\|Q12805\|A8K3I4\|D6W | 2202 | EFEMP1 | EGF-containing fibulin-like extracellular | −12.88 | 0.0256 |
| | 5D2\|Q59G97\|B2R6M6 | | | matrix protein 1 isoform b variant\|EGF- | | |
| | | | | containing fibulin-like extracellular matrix | | |
| | | | | protein 1\|cDNA, FLJ93024, highly similar | | |
| | | | | to Homo sapiens EGF-containing fibulin- | | |
| | | | | like extracellular matrix protein 1 | | |
| | | | | (EFEMP1), transcript variant 1, | | |
| | | | | mRNA\|cDNA FLJ77823, highly similar to | | |
| | | | | Homo sapiens EGF-containing fibulin-like | | |
| | | | | extracellular matrix protein 1, transcript | | |
| | | | | variant 3, mRNA | | |
| IPI00103871 | Q9NWJ8\|A8K154\|Q8TEG1\|Q8WZ75\|Q9 | 54538 | ROBO4 | Roundabout homolog 4 | −11.90 | 0.0291 |
| | 6JV6\|Q9H718\|Q14DU7 | | | | | |
| IPI00009793 | Q53GX9\|Q9NZP8 | 51279 | C1RL | Complement C1r subcomponent-like | −11.74 | 0.0142 |
| | | | | protein | | |
| IPI00019157 | D3DW77\|Q92675\|Q6UVK1 | 1464 | CSPG4 | Chondroitin sulfate proteoglycan 4 | −11.68 | 0.0185 |
| IPI00006971 | Q2M2V5\|Q9HCU0\|Q96KB6\|Q3SX55 | 57124 | CD248 | Endosialin | −11.35 | 0.0186 |
| IPI00009276 | Q14218\|Q9ULX1\|Q96CB3\|B2RC04\|Q9U | 10544 | PROCR | Endothelial protein C receptor | −10.91 | 0.0332 |
| | NN8\|Q6IB56 | | | | | |
| IPI00553177 | E9KL23\|Q0PVP5\|Q53XB8\|Q96BF9\|B2R | 5265 | SERPINA1 | Epididymis secretory sperm binding | −9.59 | 0.0265 |
| | DQ8\|Q13672\|Q5U0M1\|Q7M4R2\|P01009\| | | | protein Li 44a\|Alpha-1-antitrypsin | | |
| | Q9P1P0\|Q9UCM3\|A6PX14\|Q9UCE6\|Q9 | | | | | |
| | 6ES1\|Q86U19\|Q86U18 | | | | | |
| IPI00032293 | D3DW42\|B2R5J9\|P01034\|E9RH26\|Q6F | 1471 | CST3 | Cystatin-C\|Cystatin C | −9.16 | 0.0021 |
| | GW9 | | | | | |
| IPI00045512 | Q69YJ3\|Q5TYR7\|Q96RW7\|Q96DN8\|Q9 | 83872 | DKFZp762L | Hemicentin 1\|cDNA FLJ14438 fis, clone | −9.04 | 0.0171 |
| | 6SC3\|Q5TCP6\|Q96DN3\|Q96K89\|A6NG | | 185\|HMCN1 | HEMBB1000317, weakly similar to | | |
| | E3 | | | FIBULIN-1, ISOFORM D\|Putative | | |
| | | | | uncharacterized protein | | |
| | | | | DKFZp762L185\|Hemicentin-1 | | |
| IPI00306322 | Q14052\|Q548C3\|Q66K23\|P08572\|Q5VZ | 1284 | COL4A2 | cDNA FLJ56433, highly similar to | −7.50 | 0.0264 |
| | A9\|B4DH43 | | | Collagen alpha-2(IV) chain\|Collagen | | |
| | | | | alpha-2(IV) chain | | |
| IPI00295414 | P39059\|B3KTP7\|Q5T6J4\|Q9Y4W4\|Q9U | 1306 | COL15A1 | Collagen alpha-1(XV) chain\|cDNA | −6.84 | 0.0135 |
| | DCS | | | FLJ38566 fis, clone HCHON2005118, | | |
| | | | | highly similar to Collagen alpha-1(XV) | | |
| | | | | chain | | |
| IPI00168728 | Q8NF17 | | FLJ00385 | FLJ00385 protein | −6.63 | 0.0282 |
| IPI00289983 | Q96QM0\|D3DNC6\|Q96KY0\|P15309\|Q9 | 55 | ACPP | Prostatic acid phosphatase | −6.55 | 0.0073 |
| | 6QK9 | | | | | |
| IPI00027482 | B2R9F2\|P08185\|Q7Z2Q9\|A8K456 | 866 | SERPINA6 | Corticosteroid-binding globulin\|cDNA, | −6.54 | 0.0256 |
| | | | | FLJ94361, highly similar to Homo sapiens | | |
| | | | | serine (or cysteine) proteinase inhibitor, | | |
| | | | | clade A(alpha-1 antiproteinase, | | |
| | | | | antitrypsin), member 6 (SERPINA6), | | |
| | | | | mRNA | | |
| IPI00218851 | | | | | −6.17 | 0.0228 |
| IPI00186826 | B5A972\|B5A970\|Q96L35 | 2050 | EPHB4 | EPH receptor B4, isoform CRA_b\|Soluble | −6.14 | 0.0396 |
| | | | | EPHB4 variant 1\|Soluble EPHB4 variant 3 | | |
| IPI00292130 | A8K981\|Q9U1X8\|Q07507\|Q8N4R2 | 1805 | DPT | Dermatopontin | −5.86 | 0.0022 |
| IPI00218413 | Q96EM9\|B7Z7C9\|B2R865\|P43251 | 686 | BTD | Biotinidase\|cDNA FLJ50907, highly | −5.65 | 0.0416 |
| | | | | similar to Biotinidase (EC 3.5.1.12) | | |
| IPI00896380 | P20769\|P01871 | | IGHM | Ig mu chain C region | −5.55 | 0.0308 |
| IPI00025992 | B6EU04\|Q9BY68\|Q1HE14\|P81172 | 57817 | HAMP | Hepcidin\|Hepcidin antimicrobial peptide | −5.49 | 0.0484 |
| IPI00305457 | Q9P173 | | | PRO2275 | −5.23 | 0.0184 |
| IPI00000024 | B4E2D8\|Q81UP2\|Q08174 | 5097 | PCDH1 | cDNA FLJ59655, highly similar to | −4.61 | 0.0079 |
| | | | | Protocadherin-1\|Protocadherin-1 | | |
| IPI00021841 | Q9UCS8\|Q6LDN9\|Q9UCT8\|A8K866\|P0 | 335 | APOA1 | APOA1 protein\|Apolipoprotein A-I | −4.35 | 0.0233 |
| | 2647\|Q6Q785\|Q6LEJ8 | | | | | |
| IPI00922041 | B7Z538 | | | cDNA FLJ60766, highly similar to | −4.16 | 0.0058 |
| | | | | Hepatocyte growth factor-like protein | | |
| IPI00216728 | C9JJR0 | | NRXN3 | Neurexin-3-beta, soluble form | −4.16 | 0.0403 |
| IPI00013576 | Q8WVV5\|O00480 | 10385 | BTN2A2 | Butyrophilin subfamily 2 member A2 | −4.00 | 0.0141 |
| IPI00022284 | Q15216\|A1YVW6\|Q8TBG0\|Q27H91\|P0 | 5621 | PRNP | Major prion protein | −3.84 | 0.0118 |
| | 4156\|Q86XR1\|O60489\|Q5QPB4\|Q6FGR | | | | | |
| | 8\|Q15221\|Q6FGN5\|D4P3Q7\|Q96E70\|P78 | | | | | |
| | 446\|B4DDS1\|Q9UP19\|B2R5Q9\|Q5U0K3 | | | | | |
| | \|Q540C4\|Q53YK7 | | | | | |
| IPI00470360 | Q8TB15\|Q5XKC6\|Q9H9N1\|Q7Z7N8\|Q5 | 55243 | KIRREL | Kin of IRRE-like protein 1 | −3.52 | 0.0062 |
| | W0F8\|Q96J84\|Q9NVA5\|Q7Z696 | | | | | |
| IPI00292218 | B7Z557 | | | cDNA FLJ53076, highly similar to | −3.44 | 0.0270 |
| | | | | Hepatocyte growth factor-like protein | | |
| IP101025175 | | | | | −3.37 | 0.0047 |
| IPI00383732 | Q9Y509 | | VH3 | VH3 protein | −3.37 | 0.0476 |
| IPI00009794 | B1AME5\|B1AME6\|Q8NBQ3\|Q96AA1\|Q | 51150 | SDF4 | 45 kDa calcium-binding protein | −2.77 | 0.0403 |
| | 53HQ9\|B4DSM1\|B2RDF1\|Q9BRK5\|Q9 | | | | | |
| | NZP7\|Q9UN53\|Q53G52 | | | | | |
| IPI00329538 | Q9UCA3\|Q16651 | 5652 | PRSS8 | Prostasin | −2.74 | 0.0164 |
| IPI00010807 | Q9H461 | 8325 | FZD8 | Frizzled-8 | −2.57 | 0.0030 |
| IPI00784865 | Q6P558 | | IGK@ | IGK@ protein | −2.45 | 0.0131 |
| IPI00925540 | A6NLA3\|Q13350\|Q14870\|P26927\|Q6GT | 4485 | MST1 | Hepatocyte growth factor-like | −2.38 | 0.0016 |
| | N4\|A8MSX3\|Q53GN8\|B7Z250 | | | protein\|cDNA FLJ56324, highly similar to | | |
| | | | | Hepatocyte growth factor-like | | |
| | | | | protein\|Macrophage stimulating 1 | | |
| | | | | (Hepatocyte growth factor-like) variant | | |
| IPI00556655 | Q59FZ0 | | | LAMP1 protein variant | −2.38 | 0.0065 |
| IPI00016450 | Q96TD2\|Q6LCK3\|Q6LCK5\|Q6LCK4\|Q6 | 6340 | SCNN1G | Amiloride-sensitive sodium channel | −2.05 | 0.0466 |
| | LCK6\|Q93023\|A5X2V1\|P51170\|Q93026\| | | | subunit gamma\|Amiloride-sensitive | | |
| | Q93025\|Q93024\|Q93027\|P78437\|Q6PCC | | | epithelial sodium channel gamma | | |
| | 2 | | | subunit\|Amiloride-sensitive sodium | | |
| | | | | channel gamma-subunit | | |
| IPI00007257 | O94985\|Q5SR52\|Q5UE58\|Q71MN0\|A8K | 22883 | CLSTN1 | Calsyntenin-1 | 1.50 | 0.0118 |
| | 183\|Q8N4K9 | | | | | |
| IPI00744007 | | | | | 1.70 | 0.0050 |
| IPI00022830 | Q5JXA5\|Q5JXA4\|B2RD74\|Q9UI06\|A2A | 55968 | NSFL1C | NSFL1 cofactor p47 | 1.70 | 0.0140 |
| | 2L1\|Q9H102\|Q9UNZ2\|Q7Z533\|Q9NVL9 | | | | | |
| IPI00023974 | P53801\|D3DSL9\|A8K274\|Q9NS09\|B2R | 754 | PTTG1IP | Pituitary tumor-transforming gene 1 | 1.76 | 0.0070 |
| | DP7 | | | protein-interacting protein\|cDNA | | |
| | | | | FLJ78227, highly similar to Homo sapiens | | |
| | | | | pituitary tumor-transforming 1 interacting | | |
| | | | | protein (PTTG1IP), mRNA | | |
| IPI00030936 | Q5VST0\|D3DQ14\|O60745\|O60635 | 10103 | TSPAN1 | Tetraspanin-1 | 1.90 | 0.0306 |
| IPI00005733 | Q5T7S2\|Q706C0\|P36897\|Q6IR47\|Q706C | 7046 | TGFBR1 | TGF-beta receptor type-1\|Transforming | 1.92 | 0.0005 |
| | 1 | | | growth factor beta receptor I | | |
| IPI00169285 | Q8NHP8 | 196463 | PLBD2 | Putative phospholipase B-like 2 | 1.93 | 0.0040 |
| IPI00221255 | Q5MY99\|O95797\|O95796\|O95799\|O957 | 4638 | MYLK | Myosin light chain kinase, smooth muscle | 2.03 | 0.0043 |
| | 98\|Q15746\|Q7Z4J0\|Q9C0L5\|Q14844\|Q1 | | | | | |
| | 6794\|Q5MYA0\|Q9UBG5\|Q9UIT9 | | | | | |
| IPI00004440 | A8K604\|Q16849\|Q08319\|Q53QD6\|B4D | 5798 | PTPRN | cDNA FLJ55332, highly similar to | 2.10 | 0.0139 |
| | K12 | | | Receptor-type tyrosine-proteinphosphatase- | | |
| | | | | like N\|Receptor-type tyrosine-protein | | |
| | | | | phosphatase-like N\|cDNA FLJ77469, | | |
| | | | | highly similar to Homo sapiens protein | | |
| | | | | tyrosine phosphatase, receptor type, N, | | |
| | | | | mRNA | | |
| IPI00216773 | E7ESS9\|Q8IUK7 | | ALB | ALB protein | 2.22 | 0.0221 |
| IPI00293836 | Q8N3J6\|Q658Q7\|Q8IZP8\|Q3KQY9 | 253559 | CADM2 | Cell adhesion molecule 2 | 2.26 | 0.0230 |
| IPI00002666 | Q7M4M8\|P09086\|Q16648\|Q9BRS4\|Q9U | 5452 | OCT-2\| | Homeobox protein\|Oct-2 factor\|POU | 2.34 | 0.0004 |
| | MI6\|Q9UMJ4 | | POU2F2 | domain, class 2, transcription factor 2 | | |
| IPI00017557 | Q1ZYW2\|Q6PD64\|Q4G124\|Q6FHJ7\|Q6 | 6424 | SFRP4 | Secreted frizzled-related protein 4 | 2.34 | 0.0460 |
| | FHM0\|O14877\|B4DYC1\|Q05BG7 | | | | | |
| IPI00220737 | Q96CJ3\|Q16180\|B7Z8D6\|Q15829\|Q05C | 4684 | NCAM1 | cDNA FLJ54771, highly similar to Neural | 2.41 | 0.0028 |
| | 58\|P13591\|P13592\|P13593\|Q86X47\|Q59 | | | cell adhesion molecule 1, 120 kDa | | |
| | FL7\|A8K8T8\|Q16209 | | | isoform\|Neural cell adhesion molecule 1 | | |
| IPI00026154 | B4DJQ5\|P14314\|Q96BU9\|Q9P0W9\|E7E | 5589 | PRKCSH | Glucosidase 2 subunit beta\|Uncharacterized | 2.53 | 0.0008 |
| | QZ9\|Q96D06 | | | protein\|cDNA FLJ59211, highly similar to | | |
| | | | | Glucosidase 2 subunit beta | | |
| IPI00034319 | Q9NYQ9\|O60888\|Q5JXM9\|Q3B784\|A2 | 51596 | CUTA | Protein CutA | 2.55 | 0.0245 |
| | BEL4\|A2AB26\|Q5SU05 | | | | | |
| IPI00215997 | Q96ES4\|P21926\|Q5J7W6\|D3DUQ9 | 928 | CD9 | CD9 antigen | 2.61 | 0.0200 |
| IPI00016786 | P25763\|P21181\|P60953\|Q9UDI2\|Q7L8R | 998 | CDC42 | Cell division control protein 42 homolog | 2.61 | 0.0011 |
| | 5 | | | | | |
| IPI00219860 | P23468\|B1ALA0 | 5789 | PTPRD | Receptor-type tyrosine-protein phosphatase | 2.76 | 0.0437 |
| | | | | delta | | |
| IPI00018434 | Q9BUM5\|Q99816 | 7251 | TSG101 | Tumor susceptibility gene 101 protein | 2.79 | 0.0173 |
| IPI00219465 | Q9UDM0\|Q9BVI8\|P20062\|Q9UCI6\|Q9U | 6948 | TCN2 | Transcobalamin-2 | 2.79 | 0.0339 |
| | CI5 | | | | | |
| IPI00017367 | A7YIJ8 | | RDX | Radixin | 2.86 | 0.0122 |
| IPI00010290 | Q6FGL7\|Q05CP7\|P07148 | 2168 | FABP1 | Fatty acid-binding protein, liver\|FABP1 | 2.93 | 0.0039 |
| | | | | protein | | |
| IPI00017202 | Q7Z798\|Q7Z7A0\|Q7Z799\|Q9H9P2\|B2R | 140578 | CHODL | Chondrolectin | 3.00 | 0.0341 |
| | 9C0\|Q9HCY3 | | | | | |
| IPI00003101 | P01589\|B2R9M9\|A2N4P8\|Q5W007\|Q53 | 3559 | IL2RA\| | cDNA, FLJ94475, highly similar to Homo | 3.03 | 0.0085 |
| | FH4 | | IL2R | sapiens interleukin 2 receptor, alpha | | |
| | | | | (IL2RA), mRNA\|IL2R protein\|Interleukin- | | |
| | | | | 2 receptor subunit alpha\|Interleukin 2 | | |
| | | | | receptor, alpha chain variant | | |
| IPI00289831 | Q16341\|O75255\|Q15718\|Q13332\|O7587 | 5802 | PTPRS | Receptor-type tyrosine-protein phosphatase | 3.04 | 0.0328 |
| | 0\|D6W633\|Q2M3R7 | | | S\|Protein tyrosine phosphatase, receptor | | |
| | | | | type, S, isoform CRA_a | | |
| IPI00013972 | Q16574\|Q0Z7S6\|O60399\|P31997 | 1088 | CEACAM8 | Carcinoembryonic antigen-related cell | 3.05 | 0.0046 |
| | | | | adhesion molecule 8 | | |
| IPI00022937 | | | | | 3.19 | 0.0000 |
| IPI00027436 | B2R961\|P08138 | 4804 | NGFR | Tumor necrosis factor receptor superfamily | 3.23 | 0.0117 |
| | | | | member 16 | | |
| IPI00021968 | Q9Y6Q6 | 8792 | TNFRSF11A | Tumor necrosis factor receptor superfamily | 3.24 | 0.0112 |
| | | | | member 11A | | |
| IPI00027509 | B7Z747\|Q9UCJ9\|B7Z8A9\|P14780\|Q8N7 | 4318 | MMP9 | cDNA FLJ51036, highly similar to Matrix | 3.27 | 0.0218 |
| | 25\|Q9UDK2\|Q3LR70\|Q9UCL1\|F5GY52\| | | | metalloproteinase-9 | | |
| | Q9H4Z1\|B2R7V9\|Q9Y354\|B7Z507 | | | (EC3.4.24.35)\|Uncharacterized | | |
| | | | | protein\|Matrix metalloproteinase-9\|Matrix | | |
| | | | | metalloproteinase 9\|cDNA FLJ51120, | | |
| | | | | highly similar to Matrix metalloproteinase- | | |
| | | | | 9 (EC 3.4.24.35)\|cDNA FLJ51166, highly | | |
| | | | | similar to Matrix metalloproteinase-9 (EC | | |
| | | | | 3.4.24.35) | | |
| IPI00641251 | B2RDS5\|Q53HF7\|Q9NPF0\|D6W668 | 51293 | CD320 | CD320 antigen | 3.34 | 0.0078 |
| IPI00002910 | Q9H665\|Q8N5X0 | 79713 | IGFLR1 | IGF-like family receptor 1 | 3.49 | 0.0090 |
| IPI00025204 | A8K7M5\|O43866\|Q6UX63 | 922 | CD5L | CD5 antigen-like | 3.56 | 0.0014 |
| IPI00297646 | O76045\|Q16050\|Q9UML6\|Q13902\|Q140 | 1277 | COL1A1 | Collagen type I alpha 1\|Type II procollagen | 3.58 | 0.0160 |
| | 37\|Q13903\|Q8IVI5\|Q6LAN8\|P02452\|Q1 | | | gene\|Collagen, type I, alpha 1, isoform | | |
| | 3896\|Q59F64\|Q15176\|D3DTX7\|Q8N473\| | | | CRA_a\|Type I collagen alpha 1 | | |
| | Q15201\|Q14042\|Q14992\|Q9UMM7\|Q7K | | | chain\|Collagen alpha-1(I) chain | | |
| | Z30\|P78441\|Q7KZ34\|Q9UMA6 | | | | | |
| IPI00027463 | P06703\|Q5RHS4\|D3DV39\|B2R577 | 6277 | S100A6 | cDNA, FLJ92369, highly similar to Homo | 3.62 | 0.0207 |
| | | | | sapiens S100 calcium binding protein A6 | | |
| | | | | (calcyclin) (S100A6), mRNA\|Protein S100- | | |
| | | | | A6 | | |
| IPI00001754 | Q9Y624\|D3DVF0\|Q6FIB4 | 50848 | F11R | F11 receptor\|F11 receptor, isoform | 3.62 | 0.0048 |
| | | | | CRA_a\|Junctional adhesion molecule A | | |
| IPI00152418 | Q14UF3\|Q8TD141\|D3DT86\|B1AP16 | | DAF\|CD55 | CD55 antigen, decay accelerating factor for | 3.76 | 0.0388 |
| | | | | complement (Cromer blood group), | | |
| | | | | isoform CRA_g\|Decay-accelerating factor | | |
| | | | | splicing variant 4\|Decay-accelerating factor | | |
| | | | | 1a\|CD55 molecule, decay accelerating | | |
| | | | | factor for complement (Cromer blood | | |
| | | | | group) | | |
| IPI00289501 | O15240\|Q9UDW8 | 7425 | VGF | Neurosecretory protein VGF | 3.76 | 0.0102 |
| IPI00376457 | B4E0V9 | 342510 | | cDNA FLJ61198, highly similar to Homo | 3.97 | 0.0064 |
| | | | | sapiens CD300 antigen like family member | | |
| | | | | E (CD300LE), mRNA | | |
| IPI00216298 | P10599\|Q53X69\|Q9UDG5\|Q96KI3 | 7295 | TXN | Thioredoxin | 4.02 | 0.0028 |
| IPI00289334 | Q9UEV9\|Q13706\|Q9NT26\|C9JMC4\|Q6 | 2317 | FLNB | Filamin-B | 4.06 | 0.0268 |
| | MZJ1\|C9JKE6\|O75369\|Q8WXS9\|B2ZZ8 | | | | | |
| | 4\|B2ZZ85\|Q8WXT1\|Q8WXT0\|Q59EC2\| | | | | | |
| | Q8WXT2\|Q9NRB5 | | | | | |
| IPI00219365 | Q6PJT4\|P26038 | 4478 | MSN | MSN protein\|Moesin | 4.15 | 0.0033 |
| IPI00977659 | Q6S9E4\|A8K9Q3\|Q14C97\|Q9ULV1\|Q8 | 8322 | GPCR\| | Frizzled-4\|Putative G-protein coupled | 4.22 | 0.0057 |
| | TDT8 | | FZD4 | receptor | | |
| IPI00002280 | Q9UHG2\|Q4VC04 | 27344 | PCSK1N | ProSAAS | 4.47 | 0.0007 |
| IPI00303161 | Q96AP7\|Q96T50 | 90952 | ESAM | Endothelial cell-selective adhesion | 4.85 | 0.0008 |
| | | | | molecule | | |
| IPI00002435 | P26842\|B2RDZ0 | 939 | CD27 | CD27 antigen | 4.96 | 0.0003 |
| IPI00291488 | Q8WXW1\|Q6IB27\|A6PVD5\|1Q96KJ11A2 | 10406 | WFDC2 | WAP four-disulfide core domain protein 2 | 5.02 | 0.0413 |
| | A2A5\|Q14508\|Q8WXV9\|A2A2A6\|Q8W | | | | | |
| | XW0\|Q8WXW2 | | | | | |
| IPI00099110 | Q9Y4V9\|B1ARE9\|B1ARE8\|Q5JR26\|B1 | 1755 | DMBT1 | Deleted in malignant brain tumors 1 | 5.03 | 0.0038 |
| | ARF0\|Q9UGM3\|Q9UGM2\|Q59EX0\|B1A | | | protein | | |
| | RE7\|A8E4R5\|Q9UKJ4\|Q9UJ57\|Q96DU4\| | | | | | |
| | A6NDG4\|Q9Y211\|Q6MZN4\|A6NDJ5 | | | | | |
| IPI00179330 | B2RDW1\|Q9UEK8\|Q8WYN8\|Q91887\|Q | 6233 | RPS27A | Ribosomal protein S27a\|Ubiquitin-40S | 5.15 | 0.0004 |
| | 6LDU5\|P62988\|Q9BX98\|Q9UEF2\|P6297 | | | ribosomal protein S27a\|Ribosomal protein | | |
| | 9\|Q5RKT7\|Q9UPK7\|P14798\|Q9BWD6\|Q | | | S27a, isoform CRA_c | | |
| | 6LBL4\|P02248\|P02249\|Q91888\|Q9BQ77\| | | | | | |
| | Q29120\|P02250\|Q9UEG1 | | | | | |
| IPI00008239 | B7Z831 | | GPRC5B | G-protein-coupled receptor family C group | 5.22 | 0.0340 |
| | | | | 5 member B | | |
| IPI00301579 | E7EMS2\|B4DV10 | | NPC2 | Epididymal secretory protein E1\|cDNA | 5.49 | 0.0000 |
| | | | | FLJ59142, highly similar to Epididymal | | |
| | | | | secretory protein E1 | | |
| IPI00026926 | Q02747 | 2980 | GUCA2A | Guanylin | 5.53 | 0.0152 |
| IPI00019906 | B4DY23\|P35613\|Q7Z796\|Q54A51\|Q8IZ | 682 | hEMM | Basigin\|cDNA FLJ61188, highly similar to | 5.71 | 0.0082 |
| | L7 | | PRIN\|BSG | Basiginpasigin (Ok blood group), isoform | | |
| | | | | CRA_a | | |
| IPI00004901 | Q9NXI0 | | GPRC5C | G-protein-coupled receptor family C group | 6.07 | 0.0228 |
| | | | | 5 member C | | |
| IPI00019580 | B2R7F8\|P00747\|Q9UMI2\|Q15146\|Q5TE | 5340 | PLG | PLG protein\|Plasminogen\|cDNA, | 6.08 | 0.0084 |
| | H4\|Q6PA00\|B4DPH4 | | | FLJ93426, highly similar to Homo sapiens | | |
| | | | | plasminogen (PLG), mRNA\|cDNA | | |
| | | | | FLJ58778, highly similar to Plasminogen | | |
| | | | | (EC 3.4.21.7) | | |
| IPI00175092 | Q53SV6\|Q8WUU3\|Q8NC42\|Q8NBY5\|Q | 284996 | RNF149\| | Putative uncharacterized protein | 6.39 | 0.0102 |
| | 53S14\|Q8N5I8 | | LOC284996 | LOC284996\|E3 ubiquitin-protein ligase | | |
| | | | | RNF149 | | |
| IPI00103636 | Q8WXW1\|Q6IB27\|A6PVD5\|Q96KJ1\|A2 | 10406 | WFDC2 | WAP four-disulfide core domain protein 2 | 6.57 | 0.0191 |
| | A2A5\|Q14508\|Q8WXV9\|A2A2A6\|Q8W | | | | | |
| | XW0\|Q8WXW2 | | | | | |
| IPI00010182 | P08869\|Q4VWZ6\|Q53SQ7\|Q9UCI8\|P07 | 1622 | DBI | Diazepam binding inhibitor, splice form | 6.79 | 0.0021 |
| | 108\|B8ZWD8\|1Q6IB48 | | | 1D(1)\|Acyl-CoA-binding protein | | |
| IPI00922213 | Q14327\|Q7L553\|B4DTK1\|Q6PJE5\|Q9H3 | | FN1 | Putative uncharacterized protein | 7.00 | 0.0035 |
| | 82\|Q53S27\|B4DTH2 | | | FN1\|cDNA FLJ61165, highly similar to | | |
| | | | | Fibronectin\|FN1 protein\|Fibronectin | | |
| | | | | 1\|cDNA FLJ53292, highly similar to Homo | | |
| | | | | sapiens fibronectin 1 (FN1), transcript | | |
| | | | | variant 5, mRNA | | |
| IPI00290085 | Q14923\|Q8N173\|B0YIY6\|P19022 | 1000 | CDH2 | Cadherin-2 | 7.14 | 0.0137 |
| IPI00298388 | Q49A94\|Q8NCJ9\|Q96FE7\|Q86YW2\|O00 | 113791 | PIK3IP1 | Phosphoinositide-3-kinase-interacting | 8.23 | 0.0075 |
| | 318 | | | protein 1 | | |
| IPI00032325 | P01040\|Q61B90 | 1475 | CSTA | CSTA protein\|Cystatin-A | 8.67 | 0.0042 |
| IPI00010675 | Q15854\|Q03403 | 7032 | TFF2 | Trefoil factor 2 | 8.89 | 0.0247 |
| IPI00011302 | P13987\|Q6FHM9 | 966 | CD59 | CD59 antigen, complement regulatory | 10.07 | 0.0171 |
| | | | | protein, isoform CRA_b\|CD59 | | |
| | | | | glycoprotein | | |
| IPI00010343 | Q9UPR5\|B4DYQ9\|B4DEZ4 | 6543 | SLC8A2 | cDNA FLJ58526, highly similar to | 10.67 | 0.0069 |
| | | | | Sodium/calcium exchanger | | |
| | | | | 2\|Sodium/calcium exchanger 2 | | |
| IPI00013955 | Q9UE76\|Q9UE75\|Q9UQL1\|Q7Z552\|Q14 | 4582 | MUC1 | Mucin-1 | 10.89 | 0.0144 |
| | 876\|Q9Y4J2\|Q14128\|Q16437\|P13931\|P1 | | | | | |
| | 7626\|P15941\|Q16615\|P15942\|Q16442\|Q9 | | | | | |
| | BXA4 | | | | | |
| IPI00299086 | O00173\|O43391\|O00560\|B2R5Q7\|B4DU | 6386 | SDCBP | Syntenin-1\|Syndecan binding protein | 11.69 | 0.0132 |
| | H3\|Q14CP2\|B7ZLN2 | | | (Syntenin) | | |
| IPI00075248 | Q96HK3\|P02593\|P70667\|Q13942\|P9901 | 801\|808\|805 | CALM2\| | Calmodulin\|Calmodulin 1 (Phosphorylase | 12.10 | 0.0234 |
| | 4\|P62158\|B4DJ5\|Q953529\|Q61379\|Q613 | | CALM3\| | kinase, delta), isoform CRA_a | | |
| | 80 | | CALM1 | | | |
| IPI00302592 | Q5HY55\|Q5HY53\|P21333\|Q8NF52\|Q60 | 2316 | FLNA\| | Filamin-A\|Filamin A\|FLNA | 12.82 | 0.0025 |
| | FE6\|Q6NXF2\|Q81ES4 | | FLJ00119 | protein\|FLJ00119 protein | | |
| IPI00219684 | Q5VV93\|B2RAB6\|Q99957\|P05413\|Q6IB | 2170 | FABP3 | FABP3 protein\|Fatty acid-binding protein, | 12.84 | 0.0009 |
| | D7 | | | heart | | |
| IPI00009027 | Q2TBE1\|P05451\|Q0VFX1\|A8K7G6\|P11 | 5967 | REG1A | REG1A protein\|Putative uncharacterized | 13.55 | 0.0282 |
| | 379\|Q4ZG28 | | | protein REG1A\|cDNA FLJ75763, highly | | |
| | | | | similar to Homo sapiens regenerating islet- | | |
| | | | | derived 1 alpha (pancreatic stone protein, | | |
| | | | | pancreatic thread protein) (REG1A), | | |
| | | | | mRNA\|Lithostathine-l-alpha | | |
| IPI00012585 | P07686 | 3074 | HEXB | Beta-hexosaminidase subunit beta | 18.50 | 0.0494 |
| IPI00302944 | Q5VYK2\|Q71UR3\|Q5VYK1\|Q15955\|Q9 | 1303 | COL12A1 | Collagen alpha-1(XII) chain | 19.62 | 0.0256 |
| | 9716\|Q99715\|O43853 | | | | | |
| IPI00009030 | P13473\|Q16641\|D3DTF0\|Q6Q3G8\|Q995 | 3920 | LAMP2 | Lysosome-associated membrane | 21.17 | 0.0235 |
| | 34\|A8K4X5\|Q9UD93\|Q96J30 | | | glycoprotein 2 | | |
| IPI00007778 | F6X5H7\|B2RBF5\|Q5VX51\|Q5VX50\|Q8 | 1486 | CTBS | cDNA PSEC0114 fis, clone | 25.84 | 0.0045 |
| | TC97\|B3KQS3\|B4DQ98\|Q01459 | | | NT2RP2006543, highly similar to DI-N- | | |
| | | | | ACETYLCHITOBIASE (EC 3.2.1.-) | | |
| | | | | \|CTBS protein\|Di-N- | | |
| | | | | acetylchitobiase\|cDNA FLJ55135, highly | | |
| | | | | similar to Di-N-acetylchitobiase (EC | | |
| | | | | 3.2.1.-)\|cDNA, FLJ95483, highly similar to | | |
| | | | | Homo sapiens chitobiase, di-N-acetyl- | | |
| | | | | (CTBS), mRNA\|Chitobiase, di-N-acetyl- | | |
| IPI00031008 | C9J575\|Q14583\|Q15567\|Q5T7S3\|C9IYT | 3371 | TNC variant | TNC variant proteinrfenascin | 27.68 | 0.0421 |
| | 7\|C9J6D9\|C9J848\|Q4LE33\|P24821 | | protein\|TNC | | | |
| IPI00295741 | Q6LAF9\|A8K2H4\|Q503A6\|B3KQR5\|Q9 | 1508 | CTSB | Cathepsin B1cDNA FLJ78235 | 30.26 | 0.0454 |
| | 6D87\|P07858\|B3KRR5 | | | | | |
| IPI00022620 | P55000\|Q6PUA6\|Q53YJ6\|Q92483 | 57152 | SLURP1 | Secreted Ly-6/uPAR-related protein 1 | 43.85 | 0.0012 |
| IPI00014048 | Q1KHR2\|B2R589\|Q6ICS5\|Q16869\|Q168 | 6035 | RNASE1 | Ribonuclease pancreatic | 53.77 | 0.0034 |
| | 30\|D3DS06\|P07998\|Q9UCB4\|Q9UCB5 | | | | | |
| IPI00293088 | Q16302\|P10253\|Q09GN4\|Q8IWE7\|Q143 | 2548 | GAA | Lysosomal alpha-glucosidase | 54.43 | 0.0356 |
| | 51 | | | | | |
| IPI00220143 | Q75ME7\|Q0VAX6\|O43451\|Q81E24\|Q86 | 8972 | MGAM | Maltase-glucoamylase\|Maltase- | 65.83 | 0.0279 |
| | UM5 | | | glucoamylase, intestinal | | |

Bedtime (pm) samples

| | | | | | | |
|---|---|---|---|---|---|---|
| IPI00022420 | D3DR38\|P02753\|Q9P178\|Q8WWA3\|Q5 | 5950 | RBP4 | Retinol-binding protein 4 | 13.16 | 0.0087 |
| | VY24\|O43479\|O43478 | | | | | |
| IPI00019568 | P00734\|B4DDT3\|B2R7F7\|Q53H06\|Q53 | 2147 | F2 | Prothrombin B-chain\|cDNA FLJ54622, | 12.12 | 0.0383 |
| | H04\|Q9UCA1\|Q69EZ8\|Q4QZ40\|Q7Z7P3 | | | highly similar to Prothrombin (EC | | |
| | \|B4E1A7\|Q69EZ7 | | | 3.4.21.5)\|Prothrombin | | |
| IPI00555812 | Q53F31\|P02774\|B4DPP2\|Q16309\|Q1631 | 2638 | GC | Vitamin D-binding protein | 11.29 | 0.0073 |
| | 0\|Q6GTG1 | | | | | |
| IPI00010949 | Q9HAT2\|B3KPB0\|Q9HAU7\|Q8IUT9\|Q9 | 54414 | SIAE | Sialate O-acetylestemse | 7.05 | 0.0060 |
| | NT71 | | | | | |
| IPI00296992 | Q8N5L2\|P30530\|Q9UD27 | 558 | AXL | Tyrosine-protein kinase receptor UFO | 3.88 | 0.0454 |
| IPI00029275 | P08582\|Q9BQE2 | 4241 | MFI2 | Melanotransferrin | 3.43 | 0.0453 |
| IPI00003813 | Q9BY67\|Q8N2F4\|Q86WB8\|Q6MZK6 | 23705 | DKFZp | Putative uncharacterized protein | 3.13 | 0.0197 |
| | | | 686F1789\| | DKFZp686F1789\|Cell adhesion molecule 1 | | |
| | | | CADM1 | | | |
| IPI00735451 | A2KLM6 | | IGVH | Immunolgoobulin heavy chain | 2.98 | 0.0473 |
| IPI00334627 | A6NMY6 | | ANXA2P2 | Putative annexin A2-like protein | 2.77 | 0.0448 |
| IPI00023858 | | | | | 2.68 | 0.0059 |
| IPI00383032 | Q96K94\|B2RAY2\|Q8WW60\|Q8TDQ0 | 84868 | HAVCR2 | Hepatitis A virus cellular receptor 2 | 2.59 | 0.0202 |
| IPI00015525 | Q504V7\|B4E3H8\|Q6P2N2\|Q9H8L6 | 79812 | MMRN2 | Multimerin-2\|cDNA FLJ54082, highly | 2.14 | 0.0388 |
| | | | | similar to Multimerin-2 | | |
| IPI00015902 | Q8N5L4\|P09619\|A8KAM8 | 5159 | PDGFRB | cDNA FLJ76012, highly similar to Homo | 1.93 | 0.0161 |
| | | | | sapiens platelet-derived growth factor | | |
| | | | | receptor, betapolypeptide (PDGFRB), | | |
| | | | | mRNA\|Platelet-derived growth factor | | |
| | | | | receptor beta | | |
| IPI00021828 | P04080\|Q76LA1 | 1476 | CSTB | Cystatin-B\|CSTB protein | 1.60 | 0.0027 |
| IPI00029723 | D3DN90\|Q549Z0\|A8K523\|Q12841 | 11167 | FSTL1 | cDNA FLJ78447, highly similar to Homo | 1.51 | 0.0075 |
| | | | | sapiens follistatin-like 1 (FSTL1), | | |
| | | | | mRNA\|Follistatin-related protein 1 | | |
| IPI00183425 | Q8WU72\|Q9Y3F9\|Q9ULV3\|Q9Y3G0\|Q | 25792 | CIZ1 | Cip1-interacting zinc finger protein\|cDNA | 1.51 | 0.0038 |
| | 9UHK4\|A8K9J8\|Q9H868\|Q5SYW5\|B4E | | | FLJ60074, highly similar to Cip1- | | |
| | 0A3\|Q9NYM8\|Q5SYW3 | | | interacting zinc finger protein | | |
| IPI00020557 | Q59FG2\|Q07954\|Q6LAF4\|Q2PP12\|Q8IV | 4035 | LRP\|LRP1 | LRP protein\|Alpha-2 macroglobulin | −2.26 | 0.0465 |
| | G8\|Q6LBN5 | | | receptor\|Prolow-density lipoprotein | | |
| | | | | receptor-related protein 1\|Low density | | |
| | | | | lipoprotein-related protein 1 variant | | |
| IPI00006705 | P11684\|Q9UCM4\|B2R5F2\|Q6FHH3\|Q9 | 7356 | SCGB1A1 | Uteroglobin | −3.09 | 0.0305 |
| | UCM2 | | | | | |

TABLE 5D
Level 3 analysis (morning/bedtime samples and genders treated independently—girls)

| | | | | | | |
|---|---|---|---|---|---|---|
| | | | | | G- | |
| IPI | UniProt | Entrez | Gene name | Description | test | T-test |

Morning (am) samples

| IPI00029275 | P08582\|Q9BQE2 | 4241 | MFI2 | Melanotransferrin | −5.79 | 0.0252 |
|---|---|---|---|---|---|---|
| IPI00010949 | Q9HAT2\|B3KPB0\|Q9HAU7\|Q8IUT9\|Q9NT71 | 54414 | SIAE | Sialate O-acetylesterase | −4.95 | 0.0473 |
| IPI00414896 | Q9BZ46\|Q9BZ47\|B2RDA7\|E1P5C3\|Q8TCU2\| | 8635 | RNASET2 | Ribonuclease T2 | −2.33 | 0.0131 |
| | O00584\|Q5T8Q0 | | | | | |
| IPI00179185 | O00520\|Q96MX2\|Q66K79 | 8532 | CPZ | Carboxypeptidase Z | −2.02 | 0.0485 |
| IPI00021428 | P02568\|Q5T8M9\|P99020\|P68133 | 58 | ACTA1 | Actin, alpha skeletal muscle | −1.93 | 0.0250 |
| IPI00000816 | P42655\|P29360\|Q63631\|Q7M4R4\|D3DTH5\| | 7531 | YWHAE | 14-3-3 protein epsilon | −1.65 | 0.0468 |
| | Q4VJB6\|Q53XZ5\|P62258\|B3KY71 | | | | | |
| IPI00166729 | O60386\|Q5XKQ4\|P25311\|D6W5T8\|Q8N4N0 | 563 | AZGP1 | Zinc-alpha-2-glycoprotein | 2.63 | 0.0168 |
| IPI00009650 | Q5T8A1\|P31025 | 3933 | LCN1 | Lipocalin-1 | 4.43 | 0.0053 |
| Bedtime (pm) samples | | | | | | |
| IPI384938 | Q7Z351 | | DKFZp686 | Putative uncharacterized protein | −17.82 | 0.0304 |
| | | | N02209 | DKFZp686N02209 | | |
| IPI00009276 | Q14218\|Q9ULX1\|Q96CB3\|B2RC04\|Q9UNN8\| | 10544 | PROCR | Endothelial protein C receptor | −4.00 | 0.0368 |
| | Q6IB56 | | | | | |
| IPI00031121 | B3KXD3\|B3KR42\|P16870\|D3DP33\|A8K4N1\| | 1363 | CPE | cDNA FLJ45230 fis, clone | −2.96 | 0.0327 |
| | Q9UIU9 | | | BRCAN2021325, highly similar to | | |
| | | | | Carboxypeptidase E (EC | | |
| | | | | 3.4.17.10)\|Carboxypeptidase E | | |
| IPI00152871 | B3KWI4\|Q7RTN7\|Q495Q6\|Q8TF66 | 131578 | LRRC15 | cDNA FLJ43122 fis, clone | −1.93 | 0.0433 |
| | | | | CTONG3003737, highly similar to | | |
| | | | | Leucine-rich repeat-containing protein | | |
| | | | | 15\|Leucine-rich repeat-containing | | |
| | | | | protein 15 | | |
| IPI00003111 | P01594\|Q6LBV5 | | | Ig kappa chain V-I region AU\|DNA | 1.62 | 0.0240 |
| | | | | rearranged by a t(2,8) translocation | | |
| | | | | leading to Burkitt's lymphoma in the | | |
| | | | | cell line JI (clone JIp) | | |
| IPI00163563 | Q96S96\|Q8WW74\|Q5EVA1 | 157310 | PEBP4 | Phosphatidylethanolamine-binding | 1.64 | 0.0470 |
| | | | | protein 4 | | |
| IPI00009650 | Q5T8A1\|P31025 | 3933 | LCN1 | Lipocalin-1 | 2.92 | 0.0209 |
| IPI00019591 | Q53F89\|B4E1Z4 | | CFB | Complement factor B | 3.67 | 0.0386 |
| IPI00022429 | B7ZKQ5\|P02763\|Q8TC16\|Q5T539\|Q5U067 | 5004 | ORM1 | Alpha-1-acid glycoprotein 1 | 4.57 | 0.0067 |
| IPI00021447 | B3KXB7\|D3DT76\|P19961\|Q9UBH3 | 280 | AMY2B | Alpha-amylase 2B | 4.87 | 0.0477 |
| IPI00032258 | B0QZR6\|Q13160\|A7E2V2\|Q14033\|P0C0L4\| | 720\|721 | C4A variant | Complement C4-A\|C4A variant | 6.02 | 0.0480 |
| | 1B7ZVZ6\|Q6P4R1\|B2RUT6\|Q5JQM8\| | | protein\|C4A | protein\|Complement component 4A | | |
| | Q4LE82\|P01028\|Q9NPK5\|P78445\| | | | (Rodgers blood group) | | |
| | Q13906\|Q14835\|Q9UIP5 | | | | | |
| IPI00022488 | P02790\|B2R957 | 3263 | HPX | Hemopexin | 8.97 | 0.0165 |
| IPI00017601 | Q2PP18\|A8K5A4\|Q1L857\|A5PL27\|B3KTA8\| | 1356 | CP | cDNA FLJ76826, highly similar to | 9.65 | 0.0247 |
| | Q14063\|P00450\|Q9UKS4 | | | Homo sapiens ceruloplasmin | | |
| | | | | (ferroxidase) (CP), mRNA\|cDNA | | |
| | | | | FLJ37971 fis, clone CTONG2009958, | | |
| | | | | highly similar to CERULOPLASMIN | | |
| | | | | (EC 1.16.3.1)\|CP protein\|Ceruloplasmin | | |
| IPI00022417 | Q68CK4\|Q8N4F5\|P02750\|Q96QZ4 | 116844 | LRG1\| | Leucine-rich alpha-2-glycoprotein | 11.28 | 0.0205 |
| | | | HMFT1766 | | | |

In general, morning urine samples were overrepresented in differentially expressed proteins, a result largely based on the overwhelming effect of OSA on the urinary proteome of boys (FIG. 3b). This observation is not surprising given that OSA is a sleep disorder characterized by repetitive respiratory events at night that should therefore be more likely to manifest in morning urine; however, the opposite results emerged among girls, in whom bedtime urine samples yielded a higher number of candidate biomarkers (FIG. 3b). Moreover, differentially expressed proteins were highly specific for gender and sampling time, since poor overlap (˜3%) was observed in the candidate biomarkers identified in boys and girls across morning and bedtime samples (Tables 5A-D). Importantly, gender differences in the biomarkers detected could not be accounted for by differences in age, disease severity, or obesity (BMI z-score) since these parameters were not significantly different between the groups (FIG. 3c).

Taken together, the results suggest that failing to account for sampling time and gender substantially masks significant differences in protein expression associated with a disease state such as OSA. This concept is clearly illustrated by global proteomic analysis of morning urine samples with the t-test and G-test, which shows dramatic improvements in both number and statistical significance of biomarkers identified (FIG. 3d). Similar conclusions emerge at the individual protein level using dipeptidyl peptidase 4 (DPP4) as an example (FIG. 3e).

### Example 5

### Validation of Candidate Biomarkers Identified by Proteomic Analysis

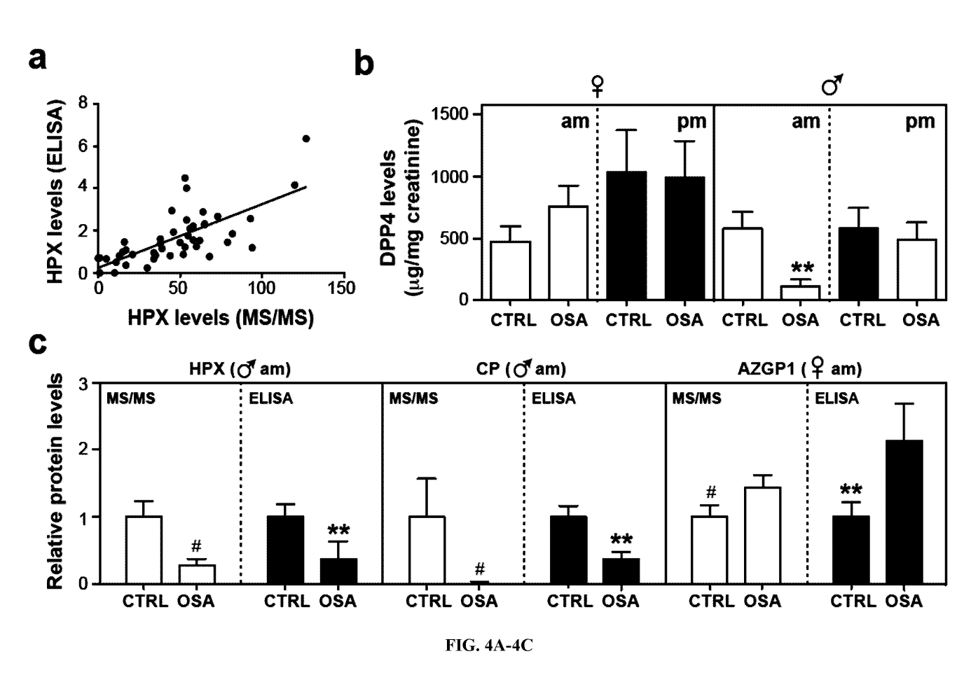

To validate the findings, the inventors used commercially available ELISA assays to measure urinary levels of four candidate biomarkers. Since protein levels in urine are highly variable, and influenced by body fluid volume, all measurements were standardized against corresponding urinary creatinine levels (Garde, 2004). ELISA measurements generally correlated well with label-free quantification by MS/MS (eg. HPX, p<0.0001, R²=0.52; FIG. 4a) and provided strong validation for gender and diurnal regulation of protein levels (e.g., DPP4; compare FIGS. 3d and 4b). In total, ELISA assays provided independent confirmations of changes in protein levels for four candidate biomarkers detected in the proteomic analyses: DPP4 (p=0.02), HPX (p=0.02), and CP (p=0.01) emerged as reliable indicators of OSA in boys, and AZGP1 (p=0.07) was identified in girls (FIG. 4b,c). Moreover, because ELISA assays involved minimal processing of urinary samples (centrifugation), while proteomic analyses required substantial processing efforts (centrifugation, IgG and ALB depletion, protein precipitation, sample digestion, etc.) the strong concordance between these two approaches further suggests that the optimized proteomic workflow approach for urine biomarker discovery is robust.

### Example 6

### Urinary Biomarkers of Pediatric OSA Map to Pathophysiological Functional Modules

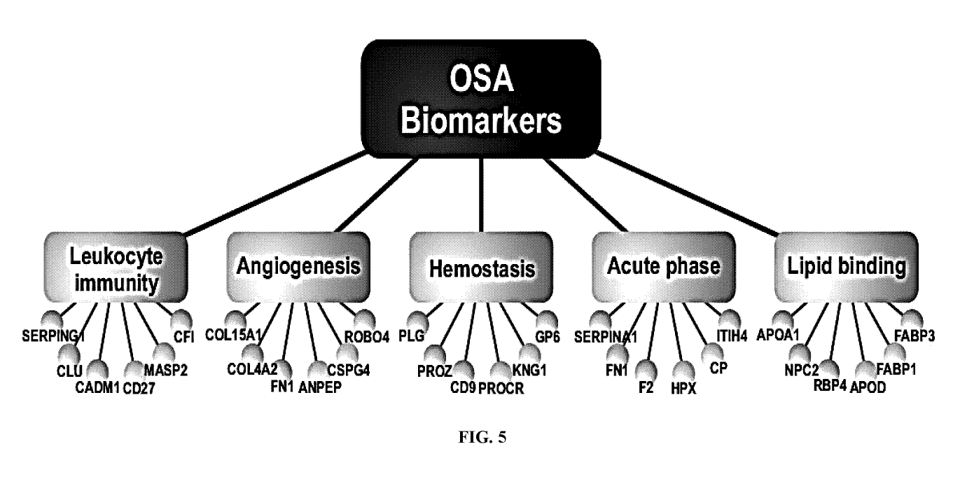

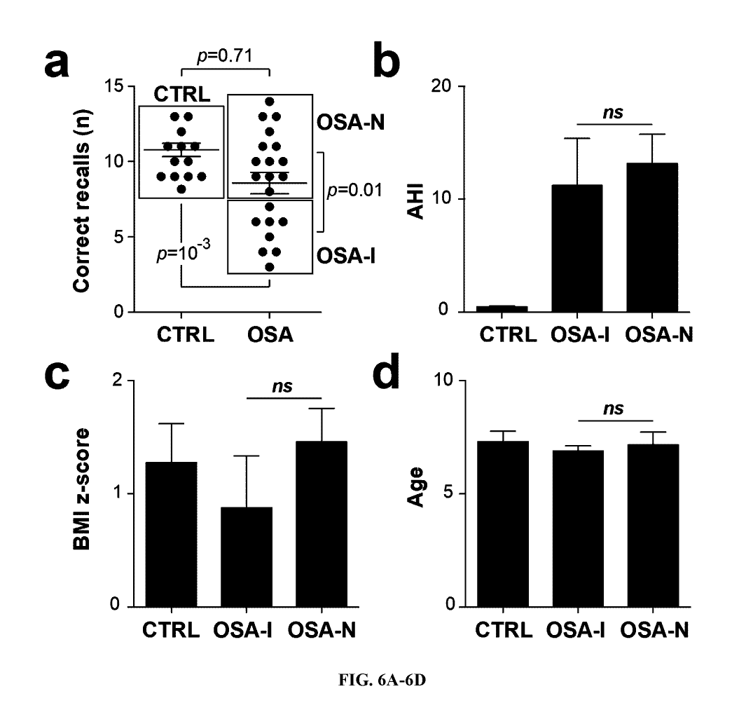

Having identified a wide range of candidate biomarkers in urine collected from children with OSA, the inventors next sought to determine whether those proteins mapped to specific functional pathways. To this end, the inventors used gene ontology analysis to organize the 192 proteins into functional modules based on biological processes and molecular function (FIG. 5). This strategy identified significant enrichment (relative to the entire human genome) in a number of functional annotations including acute phase proteins (p=8.4×10⁻⁵), angiogenesis (p=2.7×10⁻³), hemostasis (p=4.2×10⁻⁸), leukocyte immunity (p=2.4×10⁻²), and lipid binding (p=2.3×10⁻⁴). Previous studies provide evidence that all of these pathways are affected in OSA. For example, disruption in inflammatory/immune, lipid, angiogenic, and hemostatic pathways have all been reported in patients with OSA (Adedayo, 2012; Chorostowska-Wynimko, 2005; Slupsky, 2007; von Kanel, 2007).

### Example 7

### Children with OSA Demonstrate Heterogeneity in Memory Impairment

It is well established that children with OSA display neurocognitive deficits and reduced academic performance (Gozal, et al., 2010; Blunden et al., 2000; Gottlieb, et al., 2004; Kheirandish & Gozal, 2006; O'Brien, et al., 2004; Rhodes, et al., 1995; Gozal & Kheirandish-Gozal, 2007; Gozal, 1998). Declarative memory function is a critical component of academic performance and studies showed that OSA children have reduced ability to acquire, consolidate, and retrieve memories (Keirandish-Gozal, et al., 2010). To follow up on this previous work, the inventors recruited children (ages 5-12) with moderate to severe OSA along with age- and gender matched controls. The inventors assessed their sleep architecture by polysomnography and quantified their memory function using a commonly used declarative memory test previously implemented to identify neurocognitive deficits in patients with OSA (Keirandish-Gozal, et al., 2010).

In total, 33 children were recruited, with 20 subjects in the OSA group and 13 subjects in the control group. The mean age was ˜7.5 yrs. The two groups were matched for age, sex, ethnicity, level of maternal education, and obesity, as determined by BMI z-score (Table 6). In addition the incidence of physician-diagnosed asthma was similar between the two groups. Children with OSA had a significantly higher apnea-hypopnea index (AHI; p<0.0001), a measure of the severity of sleep apnea (Grigg-Damberger, et al., 2007; Redline, et al., 2007).

TABLE 6
Patient Demographics

| Group | N | Gender (M/F) | Age | AHI | BMI-z |
|---|---|---|---|---|---|
| CTRL | 13 | (7/6) | 7.8 ± 0.5 | 0.6 ± 0.1 | 1.2 ± 0.3 |
| OSA | 20 | (10/10) | 7.4 ± 0.6 | 13.1 ± 2.6 | 1.3 ± 0.3 |

The OSA group demonstrated a trend for reduced free memory recall in the morning (p=0.1). Upon closer inspection of the data, it was evident that OSA patients, but not control subjects, displayed substantial heterogeneity in their morning test performance scores (FIG. 6A). Based on this heterogeneity, the inventors classified OSA children into two phenotypes—one with normal (OSA-N, >7 recalls) and one with impaired declarative memory (OSA-I, ≤7 recalls). Importantly, OSA-N and OSA-I patients did not exhibit significant differences in OSA severity (FIG. 6B), underlying obesity (FIG. 6C), age (FIG. 6D), or gender (50% male for OSA-N and OSA-I). Thus, differences in morning memory recall in OSA-N and OSA-I patients could not be attributed to the severity of sleep disruption or any other potential confounder.

Urinary Proteomics Identifies Candidate Biomarkers of Impaired Memory in Children with OSA.

Our findings demonstrate that children with OSA may be separated into two phenotypes based on the severity of associated impairment of acquisition, consolidation, or retrieval of memories. On a molecular level, this observed phenotypic heterogeneity may be explained by variable systemic responses to OSA, which have been reported in children (Gozal, et al., 2007; Bhattacharjee, et al., 2010). The urinary proteome is largely derived from the systemic compartment and the inventors have previously shown that changes to urinary proteins can report pathophysiology in the context of OSA (Gozal, et al., 2009).

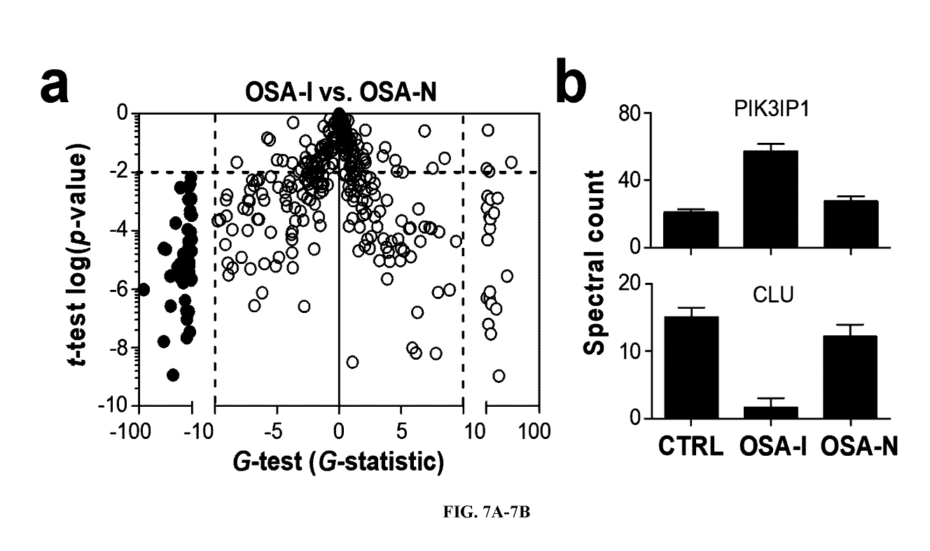

To define candidate biomarkers of memory impairment in children with OSA the inventors used liquid chromatography mass spectrometry (LC-MS/MS) to interrogate morning urine samples (first void) collected from healthy children (N=13), OSA-N(N=8) and OSA-I (N=12) patients. Urine was processed using a rigorous and reproducible workflow for proteomics analysis to identify 745 urinary proteins across all subjects. Protein levels were quantified by spectral counting (Liu, et al., 2004) and proteins that were differentially abundant between groups were identified using a combination of the G-test and t-test (Becker, et al., 2010; Becker, et al., 2010; Heinecke, et al., 2010; Almendros, et al., 2014). Using very stringent dual statistical criteria (G-test: G-statistic >10 and t-test: p<0.01) and random permutation analysis to ensure a false discovery-rate (FDR)<0.1%, the inventors identified 65 proteins that were significantly altered in OSA-I relative to OSA-N patients. (FIG. 7A). An identical approach was implemented to identify 93 proteins that were significantly altered in OSA-I relative to control subjects (data not shown). Candidate biomarkers were defined as those proteins that showed consistent increases (or decreases) in OSA-I relative to both OSA-N and CTRL subjects. Such analyses produced a list of 52 candidate biomarkers of memory impairment in children with OSA (Table 7); clusterin (CLU) and phosphoinositide-3-kinase-interacting protein 1 (PIK3IP1) are provided as two examples of proteins that met these very stringent criteria (FIG. 7B).

TABLE 7
Candidate Biomarkers of Memory Impairment in Children
with Obstructive Sleep Apnea

| | OSA-I | | OSA-I | | OSA-N | |
|---|---|---|---|---|---|---|
| | vs CTRL | | vs OSA-N | | vs CTRL | |
| Protein | G-test | T-test | G-test | T-test | G-test | T-test |
| RNASE1 | 101.5 | 2.72E−04 | 68.4 | 2.31E−03 | 3.8 | 1.32E−01 |
| COL12A1 | 45.5 | 2.82E−06 | 33.4 | 4.06E−04 | 1.0 | 3.24E−01 |
| RNASE2 | 31.6 | 1.09E−09 | 19.2 | 6.25E−05 | 1.6 | 1.06E−01 |
| CD59 | 29.1 | 1.29E−03 | 18.7 | 1.46E−02 | 1.2 | 2.35E−01 |
| FN1 | 26.9 | 2.12E−07 | 20.5 | 2.08E−05 | 0.5 | 2.72E−01 |
| AMBP | 23.2 | 4.00E−04 | 21.6 | 5.58E−04 | 0.0 | 8.47E−01 |
| FBN1 | 18.4 | 3.12E−07 | 13.4 | 7.09E−05 | 0.4 | 2.98E−01 |
| PIK3IP1 | 17.7 | 2.97E−08 | 10.8 | 1.08E−05 | 0.9 | 6.16E−02 |
| CDH1 | 17.4 | 1.19E−03 | 11.0 | 9.50E−03 | 0.8 | 1.71E−01 |
| CDH2 | 16.3 | 1.22E−04 | 13.5 | 6.27E−04 | 0.1 | 4.15E−01 |
| PLG | 16.1 | 8.13E−07 | 12.6 | 1.24E−04 | 0.2 | 3.78E−01 |
| SLURP1 | 15.0 | 2.94E−04 | 10.5 | 2.30E−03 | 0.4 | 2.74E−01 |
| FN1 cDNA | 13.7 | 6.38E−08 | 10.7 | 7.56E−06 | 0.2 | 2.81E−01 |
| FLJ53292 | | | | | | |
| TNC | 11.9 | 4.88E−05 | 11.1 | 3.15E−04 | 0.0 | 8.32E−01 |
| C1RL | −10.2 | 5.02E−05 | −10.6 | 3.17E−04 | 0.0 | 8.72E−01 |
| A1BG | −10.6 | 2.01E−05 | −16.8 | 1.07E−03 | 0.8 | 2.27E−01 |
| PGLYRP2 | −11.2 | 6.58E−03 | −13.5 | 1.55E−03 | 0.1 | 6.21E−01 |
| OSCAR | −11.3 | 2.04E−06 | −11.4 | 1.50E−05 | 0.0 | 9.81E−01 |
| AZGP1 | −12.7 | 4.86E−04 | −11.0 | 3.00E−03 | −0.1 | 7.32E−01 |
| CEL | −12.9 | 4.60E−05 | −12.8 | 1.15E−04 | 0.0 | 9.67E−01 |
| CFI | −14.0 | 8.15E−06 | −12.0 | 5.87E−05 | −0.1 | 4.40E−01 |
| CILP2 | −14.3 | 3.79E−06 | −15.3 | 2.54E−04 | 0.0 | 7.56E−01 |
| VASN | −14.6 | 6.55E−06 | −15.9 | 1.43E−04 | 0.0 | 7.36E−01 |
| PLAU | −14.6 | 3.24E−03 | −10.5 | 3.61E−03 | −0.5 | 3.77E−01 |
| SERPINA1 | −15.2 | 1.72E−07 | −16.6 | 6.25E−04 | 0.0 | 7.94E−01 |
| CD14 | −15.4 | 4.23E−05 | −17.6 | 2.80E−03 | 0.1 | 6.83E−01 |
| LRP2 | −15.7 | 1.17E−03 | −16.1 | 3.10E−03 | 0.0 | 9.41E−01 |
| CLU | −15.8 | 4.03E−06 | −11.6 | 4.83E−04 | −0.4 | 2.11E−01 |
| FGA | −16.1 | 3.09E−03 | −24.9 | 1.77E−03 | 1.3 | 2.10E−01 |
| NID1 | −16.5 | 8.19E−06 | −18.3 | 1.62E−04 | 0.1 | 6.78E−01 |
| APOD | −17.0 | 1.17E−05 | −11.5 | 1.81E−03 | −0.6 | 2.30E−01 |
| SERPING1 | −17.0 | 1.08E−04 | −14.7 | 1.67E−04 | −0.1 | 5.72E−01 |
| CADM4 | −18.2 | 9.29E−08 | −11.3 | 3.58E−04 | −1.1 | 2.68E−02 |
| CP | −18.3 | 2.22E−08 | −26.0 | 7.84E−04 | 1.0 | 1.57E−01 |
| IGHA1 | −19.3 | 1.84E−07 | −15.0 | 2.29E−04 | −0.3 | 2.49E−01 |
| PGLYRP1 | −21.7 | 4.20E−07 | −21.5 | 3.10E−04 | 0.0 | 9.76E−01 |
| ROBO4 | −22.5 | 2.07E−06 | −15.0 | 1.20E−04 | −0.9 | 1.06E−01 |
| SERPINA5 | −24.6 | 1.60E−05 | −20.2 | 3.85E−04 | −0.2 | 5.06E−01 |
| MASP2 | −24.7 | 1.67E−06 | −17.6 | 4.18E−04 | −0.8 | 1.39E−01 |
| HPX | −28.9 | 2.40E−06 | −26.3 | 6.67E−05 | −0.1 | 6.71E−01 |
| IGHV4-31 | −29.3 | 2.94E−03 | −24.5 | 6.38E−03 | −0.2 | 7.21E−01 |
| IGHG1 | −29.5 | 3.56E−06 | −20.1 | 7.45E−04 | −1.3 | 9.09E−02 |
| MXRA8 | −29.7 | 7.39E−06 | −24.6 | 5.00E−05 | −0.3 | 4.86E−01 |
| AMY1C; | −34.3 | 5.75E−06 | −30.5 | 1.03E−05 | −0.1 | 5.77E−01 |
| AMY1A; | | | | | | |
| AMY1B; | | | | | | |
| AMY2A | | | | | | |
| COL6A1 | −37.3 | 1.83E−04 | −23.7 | 1.73E−04 | −1.6 | 2.28E−01 |
| EGF | −42.1 | 1.18E−09 | −27.9 | 7.42E−05 | −1.6 | 6.32E−02 |
| PROCR | −45.7 | 2.69E−07 | −38.4 | 2.76E−05 | −0.4 | 3.73E−01 |
| PIGR | −46.5 | 2.86E−06 | −49.4 | 3.64E−06 | 0.1 | 7.61E−01 |
| ITIH4 | −54.2 | 2.30E−05 | −34.4 | 2.64E−04 | −2.7 | 1.01E−01 |
| CUBN | −57.4 | 1.62E−08 | −48.7 | 1.12E−04 | −0.5 | 3.92E−01 |
| LMAN2 | −57.4 | 2.50E−05 | −59.2 | 1.59E−05 | 0.0 | 9.00E−01 |
| TF | −91.4 | 9.76E−07 | −45.8 | 2.35E−04 | −8.6 | 1.71E−03 |

Proteins were quantified by spectra counting
Statistical significance was assessed by t-test (p < 0.01) and G-test (G-statistic >10 or <−10);
positive G = up-regulated in first stample relative to second,
negative G = down-regulated in first sample relative to second

Interestingly, informatics analysis of the candidate biomarkers identified significant enrichment in the inflammatory response (p=10⁻⁶; Fisher's exact test with Benjamini-Hochberg correction). These findings are consistent with previous work that demonstrated a strong correlation between plasma C-reactive protein levels (a marker of inflammation) and neurocognitive function in children with OSA (Gozal, et al., 2007). Together, these data suggest that the presence of OSA-associated inflammation may predispose children to memory deficits and neurocognitive impairments.

ELISA Assays Validate Proteomics Data and Enable High Throughput Clinical Screening.

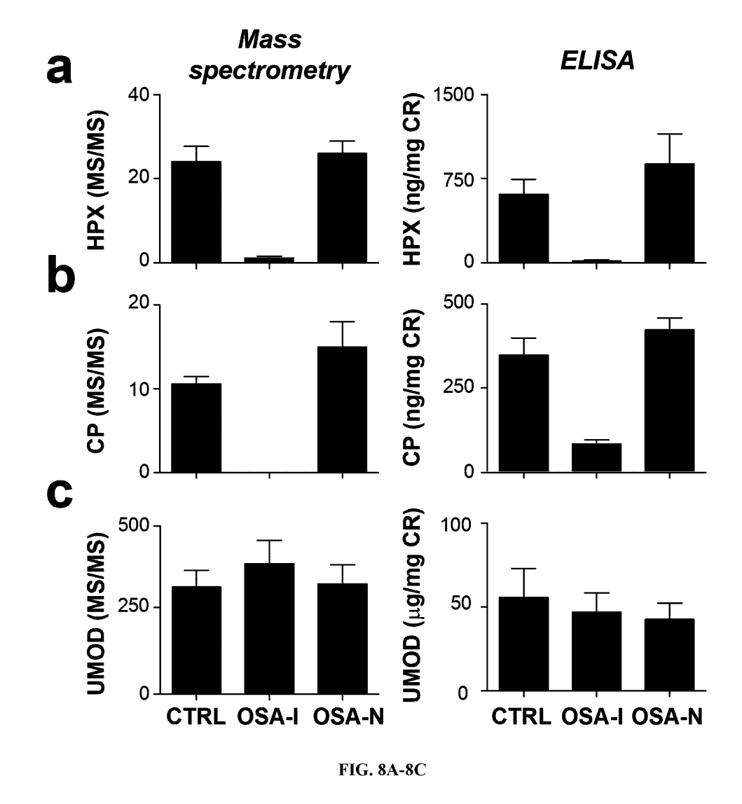

To validate the mass spectrometric findings, the inventors used commercially available ELISA assays to measure urinary levels of hemopexin (HPX) and ceruloplasmin (CP), 2 candidate biomarkers of memory impairment in children with OSA. As a control, the inventors also quantified urinary levels of uromodulin, a protein whose levels in CTRL, OSA-I and OSA-N subjects were unchanged. Since protein levels in urine are highly variable, and influenced by body fluid volume, all measurements were standardized against corresponding urinary creatinine levels (Garde, et al., 2004). ELISA assays reproduced the regulatory patterns of HPX, CP, and UMOD predicted by mass spectrometric analyses (FIG. 8A-C). These findings provide strong validation for the proteomic and statistical methods for identifying candidate biomarkers of memory impairment in children with OSA. Moreover, the development of ELISA assays for HPX and CP enable high throughput clinical screening.

### Example 8

### Develop High Throughput ELISA Assays for Candidate Urinary Biomarkers of Declarative Memory Deficit in Children with OSA

Using discovery-based proteomics, the inventors identified 52 candidate biomarkers of declarative memory impairment in children with OSA and further validated the protein abundance (measured by mass spectrometry) changes for two of these proteins (HPX and CP) by ELISA. Validated candidate biomarkers will be used to develop a multivariate classifier (a combinatorial panel) whose predictive power will be interrogated in a larger, independent patient cohort using high throughput ELISA assays.

Experimental Design.

Studies will use pre-existing urine samples (stored at −80° C.) that were analyzed by proteomics to validate candidate biomarkers that distinguish OSA-I patients from CTRL and OSA-N subjects (see FIGS. 6-8). Based on the statistical significance and magnitude of the change in urinary protein levels (assessed by the t-test and G-test), availability of ELISA-compatible antibodies and/or kits, and biological function, the inventors have selected 10 candidates for initial testing (Table 8).

TABLE 8
Candidates for ELISA assay development

| Protein | G-test * | t-test | Function |
|---|---|---|---|
| KNG1 | −91 | 10⁻⁶ | Coagulation |
| PIGR | −46 | 10⁻⁷ | Immunity |
| PROCR | −42 | 10⁻⁹ | Coagulation |
| HPX ** | −29 | 10⁻⁶ | Iron metabolism |
| CP ** | −18 | 10⁻⁸ | Iron metabolism |
| RNASE1 | 101 | 10⁻⁶ | Nucleotide metabolism |
| COL12A1 | 46 | 10⁻⁷ | Extracellular matrix |
| CD59 | 29 | 10⁻⁹ | Complement activation |
| APOH | 17 | 10⁻⁶ | Lipid metabolism |
| CTBS | 15 | 10⁻⁸ | Carbohydrate metabolism |

* negative G-test = reduced in OSA-I relative to OSA-N
** urinary ELISA assays already developed

Quantification of Urinary Proteins by ELISA.

Urine proteins will be quantified using commercially available ELISAs for CP, PROCR, APOH, KNG1 (Assaypro), HPX (Innovative Research, Inc.), PIGR, RNASE1, COL12A1, CTBS (USCN Life Science), CD59 (Neobiolab), and creatinine (Abcam) according to the manufacturer's protocols. To account for variable hydration states, protein levels will be standardized to urine creatinine levels (Garde, et al., 2004) and statistical significance between the groups will be assessed by a two-tailed, Student's t-test. This will corroborate that the previously identified differentiation between case and control samples (i.e., OSA-I and OSA-N) is still present when the candidate biomarkers are measured using an independent technology (i.e., ELISA). The inventors have already confirmed the proteomics findings for HPX, CP, and UMOD in previously analyzed patients (FIG. 8).

### Example 9

### Determine the Predictive Power of Candidate Urinary Biomarkers in a Larger, Independent Cohort of Children with OSA

Children going through the Pediatric Sleep Laboratory at the University of Chicago will undergo polysomnography, memory testing, and provide urine samples for biochemical analysis. Initial measurements will focus on HPX and CP, which the inventors have already validated by ELISA. Additional candidate biomarkers will be tested as ELISA assays are developed in Example 8.

Experimental Design.

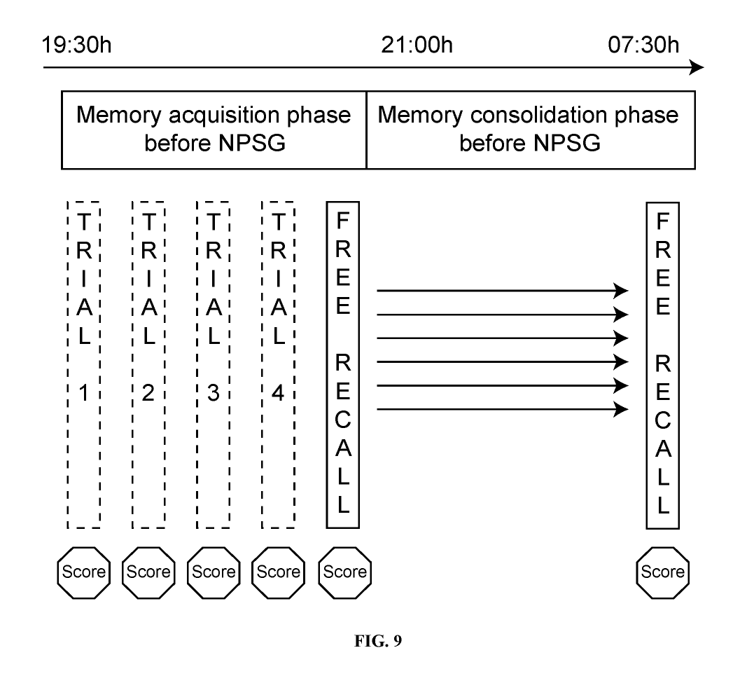

Children fulfilling the inclusion criteria for this study will be recruited according to the institutional human studies guidelines. All participating children will be admitted to the Pediatric Sleep Laboratory at the University of Chicago for an overnight stay. OSA severity will be assessed by polysomnography, declarative memory will be assessed by the validated pictorial memory test (Kheirandish-Gozal, et al., 2010), and morning urine samples will be collected for biochemical analysis (FIG. 9). Initial measurements will focus on HPX and CP, as the inventors have already developed ELISA assays for these candidate biomarkers. Additional candidates will be tested as ELISA assays are developed.

Patient Selection.

The population targeted for this study will consist of children ages 5-12 years who are referred for clinical evaluation of snoring at the University of Chicago Sleep Medicine Center. This facility evaluates in excess of 1,250 children per year, and approximately 80% of these have snoring and suspected sleep disordered breathing as their primary reason for clinical referral. Healthy children (n=50) will be recruited from schools or well-child clinics to serve as controls. Inclusion criteria for children with OSA will include children who snore frequently >3 times/week using the extensively validated questionnaire (Spruyt-Gozal, 2012). Exclusion criteria for control and OSA children will include the presence of significant genetic or craniofacial syndromes, diabetes, cystic fibrosis, cancer, or treatment with oral corticosteroids, antibiotics, or anti-inflammatory medications. Additionally, participants will be excluded if they suffer from any chronic psychiatric condition, have a genetic syndrome known to affect cognitive abilities, or are receiving medications that are known to interfere with memory or sleep onset or sleep architecture.

Overnight Polysomnography.

All participating children will undergo an overnight polysomnography (PSG) using state of the art methods (Montgomery-Downs, 2006). The severity of OSA will be quantified by the obstructive apnea-hypopnea index (AHI), which is defined as the number of obstructive apneas and hypopneas per hour of total sleep time (Grigg-Damberger, et al., 2007; Redline, et al., 2007).

Memory Recall Test.

To assess memory recall, a blinded investigator will implement a common method (Kheirandish-Gozal, et al., 2010) to evaluate children with OSA (FIG. 9). Children will be shown a series of 26 colorful animal pictures, all of which are highly familiar to children (e.g. dog, cat, chicken, lion, elephant, giraffe, horse, cow, camel, fish, butterfly, etc.). Subjects will be allowed to look at each animal picture for 10 s. The child will initially identify the animal and then the investigator will also name each animal (while pointing them out) as further corroboration of the adequate recognition of the animal in each picture. After all pictures have been shown, the book will be closed and the subjects will be given 2 min to freely recall any of the animals they could remember without looking at the pictures. One point will be awarded for every correct answer, and points will not be deducted for wrong answers and subjects will be told that they are allowed to repeat animal names if they wished to do so. After the first trial, the subjects will be allowed to look at the pictures again and go over the animal names. This process will be repeated a total of four times in the evening (acquisition phase), followed by a first recall test 10 min after completion of the fourth trial. During this 10-min interval the child will be allowed to watch TV. The morning after the sleep study, within 10-15 min of awakening, the subjects will be asked to recall the pictures that they remembered from the previous evening's trials, and the morning score will be calculated.

Urine Collection and Processing.

Mid-stream urine specimens will be collected as the first void in the morning after awakening or in the evening. To minimize protein degradation, samples (20 mL) will be immediately transferred into tubes containing the serine protease inhibitor PMSF (2 mM final concentration), and stored at −80° C. until analysis (Gozal, et al., 2009).

Development of a Multivariate Classifier.

Different multivariate classifiers (groups of candidate biomarkers) will be built using ELISA measurements that sequentially incorporate corroborated proteins to evaluate their complementary contribution to classifier performance. These multivariate classifiers will be constructed using linear discriminant analysis (McLachlan, 2004), which assigns a numerical weight to each biomarker that reflects its contribution (within the aggregated classifier score) to jointly differentiate OSA-I from OSA-N subjects.

Evaluation of Candidate Biomarkers and Classifier Performance.

The sensitivity and specificity of each individual candidate biomarker or each multivariate classifier (group of biomarkers) will be calculated on the basis of tabulating the number of correctly and incorrectly classified samples (ie. OSA-I versus OSA-N). Receiver operating characteristic (ROC) plots will be obtained by plotting all sensitivity values on the y-axis against their equivalent (1-specificity) values on the x-axis for all available thresholds. The overall accuracy of each test will be evaluated by area under the curve, as it provides a single measure that is not dependent on a particular threshold (Fawcett, et al., 2006). Unadjusted p-values will be calculated on the basis of the natural logarithm-transformed intensities and the Gaussian approximation to the t distribution. Statistical adjustment for multiple testing will be performed by the method described by Reiner and colleagues (Reiner, et al., 2003).

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and any others listed herein, to the extent that they provide exemplary procedural or other details supplementary to those set forth herein, are specifically incorporated herein by reference in their entirety.
Adachi, et al., Genome Biol. 7(9): R80, 2006.
Adedayo, et al., “Obstructive sleep apnea and dyslipidemia: evidence and underlying mechanism. Sleep & Breathing, 2012.
Becker, et al., Cell Metab. 11(2): 125-135, 2010.
Becker, et al., PLoS ONE. 7(3): e33297, 2012.
Benjamini, et al., J Roy Stat Soc B Met. 57(1):289-300, 1995.
Chen, et al., Proteomics: Clin Apps. 1: 577, 2007.
Chorostowska-Wynimko, et al., J Physiol Pharmacol. 56(Suppl 4): 71, 2005.
Christensen, et al., PLoS Genet. 5(8): e1000602, 2009.
Escudero, et al., “Machine learning-based method for personalized and cost-effective detection of Alzheimer's disease.” IEEE transactions on biomedical engineering. 2012.
Garde, et al., Ann Occup Hyg. 48(2): 171-9, 2004.
Gozal, et al., Am J Respir Crit Care Med. 177(10): 1142-1149, 2008.
Gozal, et al., Am J Respir Crit Care Med. 177(4): 369-75, 2008.
Gozal, et al., Am J Respir Crit Care Med. 180(12): 1253-1261, 2009.
Gozal, et al., Ann NY Acad Sci. 1264(1): 135-141, 2012.
Gozal, et al., Curr Op Ped. 20(6): 654-8, 2008.
Gozal, et al., Pediatrics. 126(5): e1161-7, 2010.
Gozal, et al., Sleep Med. 11(7): 708-13, 2010.
Heinecke, et al., Bioinformatics. 26(12): 1574-5, 2010.
Huttenhain, et al., Sci Transl Med. 4(142): 142ra94, 2012.
Iber, et al., The AASM manual for the scoring of sleep and associated events: rules, terminology, and technical specifications. 2007.
Keller, et al., Anal Chem. 74(20): 5383-92, 2002.
Kentsis, Ped Int: Off J Japan Ped Soc. 53(1): 1-6, 2011.
Kersey, et al., Proteomics. 4: 1985-8, 2004.
Kim, et al., Respir Physiol Neurobiol. 178(3): 465-74, 2011.
Kushnir, et al., J Biomol Tech. 20(2): 101-8, 2009.
La Thangue, et al., Nat Rev Clin Oncol. 8(10): 587-96, 2011.
Leary, et al., Sci Transl Med. 2: 20ra14, 2010.
Liu, et al. Anal Chem. 76(14): 4193-201, 2004.
Maere, et al., Bioinformatics. 21(16): 3448-9, 2005.
Montgomery-Downs, et al., Pediatrics. 117(3): 741-53, 2006.
Nesvizhskii, et al., Anal Chem. 75(17): 4646-58, 2003.
Old, et al. Mol Cell Proteomics. 4: 1487, 2005.
Rauch, et al., J Proteome Res. 5: 112, 2006.
Riaz, et al. Diabetes Tech Thera. 12(12): 979-88, 2010.
Siwy, et al., Proteomics. 5(5-6): 367-74, 2011.
Slupsky, et al., Anal Chem. 79(18): 6995-7004, 2007.
Soggiu, et al., “A discovery-phase urine proteomics investigation in type 1 diabetes” Acta Diabetol. (In press), 2012.
Stratz, et al., Cardiol Rev. 20: 111, 2012.
Thongboonkerd, et al., J Proteome Res. 5(1): 183-91, 2006.
Verrills, et al., Am J Respir Crit Care Med. 183(12): 1633-43, 2011.
Von Kanel, et al., Chest. 131(3): 733-9.
Zengi, et al., Clin Chem Lab Med: CCLM/FESCC. 50: 529, 2012.
Zimmerli, et al., Mol Cell Proteomics. 7(2): 290-8, 2007.
Zoidakis, et al., Mol Cell Proteomics. 11(4): M111.009449, 2012.
Zurbig, et al., Diabetes. 61(12): 3304-13, 2012.

1. A method for measuring expression in a subject with symptoms of obstructive sleep apnea (OSA) comprising:
a) measuring from a biological sample from the subject the expression levels of CD14, CTSB, HPX, DPP4, TTR, DEFB1|HBD1, FABP3, CP, and AZGP1.

2. (canceled)

3. The method of claim 1, comprising comparing the measured expression levels to control or reference levels.

4. The method of claim 1, wherein the control or reference levels represent the expressions levels indicative of a subject with OSA.

5. The method of claim 1, wherein the control or reference levels represent the expression levels indicative of a subject known not to have OSA.

6. The method of claim 1, wherein the measured expression levels are standardized against the level of expression of a corresponding standard product in the sample.

7. (canceled)

8. The method of claim 1, wherein the one or more products further include one or more of the following genes HPX, DPP4, CP, and AZGP1.

9. The method of claim 1, wherein the control or reference levels are sex-matched with the subject.

10. The method of claim 1, further comprising obtaining the biological sample from the subject.

11. The method of claim 1, wherein the sample is a urine sample.

12. The method of claim 6, wherein the corresponding standard product is urinary creatine.

13-23. (canceled)

24. The method of claim 1, further comprising performing a sleep study on the subject with measured expression levels that are elevated compared to expression levels indicative of a subject known not to have OSA.

25-27. (canceled)

28. The method of claim 24, wherein the sleep study comprises using an actigraph.

29-33. (canceled)

34. A method for evaluating obstructive sleep apnea in a subject comprising subjecting the subject to a sleep study after a urine sample from the subject is measured for expression levels of CD14, CTSB, HPX, DPP4, TTR, DEFB1|HBD1, FABP3, CP, and AZGP1 and at least one measured expression level is determined to be increased or decreased as compared to a control or reference level indicative of a subject known not to have sleep apnea.

35-38. (canceled)

39. The method of claim 1, wherein the subject is a child.

40. The method of claim 1, further comprising calculating a risk score.

41. The method of claim 1, wherein calculating a risk score comprises applying model coefficients to each of the levels of expression.

42. The method of claim 1, wherein expression levels of RNA transcripts are measured using an amplification or hybridization assay.

43. A method for measuring expression in a subject with symptoms of obstructive sleep apnea (OSA) comprising: measuring expression levels of biomarkers, wherein the biomarkers consist of CD14, CTSB, HPX, DPP4, TTR, DEFB1|HBD1, FABP3, CP, and AZGP1 in a biological sample from the subject

44. The method of claim 44, wherein the method further comprises measuring the expression levels of one or more controls.
